(19) United States
(12) Patent Application Publication
(10) Pub. No.: US 2018/0305331 A1
(43) Pub. Date: Oct. 25, 2018

(54) PYRIDONE DERIVATIVES AND THEIR USE AS KINASE INHIBITORS

(71) Applicant: SELVITA S.A.ó, Kraków (PL)

(72) Inventors: Tomasz RZYMSKI, Kraków (PL); Mariusz MILIK, Kraków (PL); Krzysztof BRZOZKA, Kraków (PL); Charles-Henry FABRITIUS, Kraków (PL); Katarzyna KUCWAJ-BRYSZ, Kraków (PL); Urszula KULESZA, Bialystok (PL); Ewelina WINCZA, Kraków (PL); Agnieszka DREAS, Poznan (PL); Michal GALEZOWSKI, Skierniewice (PL)

(73) Assignee: SELVITA S.A., Kraków (PL)

(21) Appl. No.: 15/770,074

(22) PCT Filed: Oct. 20, 2016
(86) PCT No.: PCT/EP2016/075269

§ 371 Date: Apr. 20, 2018

(30) Foreign Application Priority Data
Oct. 22, 2015 (EP) 15460097.7

(51) Int. Cl. C07D 401/04; C07D 405/14; C07D 401/14; C07D 417/14; C07D 409/14; C07D 471/04; C12N 9/12

(52) U.S. Cl. CPC C07D 401/04; C12Y 207/11; C12Y 207/12; C07D 405/14; C12N 9/12; C07D 417/14; C07D 409/14; C07D 471/04; C07D 401/14

(57) ABSTRACT

Disclosed in the present application is a compound of formula (I) as defined herein as well as a pharmaceutical composition comprising said compound. Further disclosed in the present application is the use of such pharmaceutical compositions for treating diseases, namely inter alia for use in the treatment of cancer, metabolic, inflammatory, autoimmune and viral diseases. The compounds disclosed herein are inhibitors of MNK1 and/or MNK2 kinases.

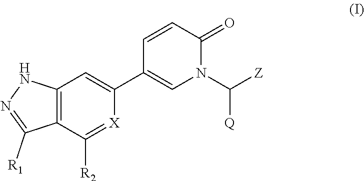

### FIELD OF THE INVENTION

The present invention is concerned with a compound of formula (I) as defined herein or a salt, stereoisomer, tautomer or N-oxide thereof. The present invention is further concerned with the use of a compound of formula (I) as defined herein or a salt, stereoisomer, tautomer or N-oxide thereof as medicament. A pharmaceutical composition comprising the compound of formula (I) as defined herein or a salt, stereoisomer, tautomer or N-oxide thereof is thus also subject of the present invention. Specific diseases to be treated with such a pharmaceutical composition are also given in the present invention. Finally, the present invention is concerned with the use of a compound of formula (I) as defined herein or a salt, stereoisomer, tautomer or N-oxide thereof as defined herein below.

### BACKGROUND OF THE INVENTION

Protein kinases are important enzymes involved in many different cellular functions and an aberrant activity of these enzymes is implicated in various diseases. Mitogen-activated protein kinases (MAPKs) can be activated in response to various signals including growth factors, environmental stress and cytokines. MAPKs are involved in the regulation of differentiation, cell cycle control, survival and programmed cell death. In response to stimulation, MAPKs activate downstream target proteins including transcriptional targets and kinases referred to as the “MAPK-activated protein kinases family” (MAPKAPK). This family comprises inter alia the MSK proteins (MAPK signal-integrating kinase/MAPK-interacting kinases), which comprise two members (MNK1 [MNK1a or MNK1b] and MNK2 [MNK2a or MNK2b]). MNK1 and MNK2 are activated directly by both ERK and p38 MAPK pathways, which phosphorylate threonine sites in the activation loop. In contrast to MNK1, which are activated by various stimuli depending on the context, MNK2 shows rather high basal activity and is hardly affected by changes in MAPK activity.

Several studies indicate that the major substrate for activated MNKs is the eukaryotic translation initiation factor 4E (eIF4E), also known as cap-binding protein. eIF4E is as a central component of the eIF4F complex, binds to the 5′ m7GpppN cap structure on mRNAs and plays an essential role in translation that is cap-dependent. Several studies consistently indicate that either MNK1 or MNK2 phosphorylates eIF4E at serine 209 in vitro and in vivo (Ueda, Watanabe-Fukunaga, Fukuyama, Nagata, & Fukunaga, 2004; Waskiewicz et al., 1999). The functional role of Ser209-phosphorylation in translation-initiation remains unclear, but both stimulatory and inhibitory effects of the Ser209-phosphorylation on the translation rates have been reported (Goetz, Thiele, & Pendergast, 2011; Jackson, Hellen, & Pestova, 2010; Müller et al., 2013).

eIF4E can act as a bona fide oncogene in vitro and in vivo. Transgenic expression of eIF4e results in neoplastic transformation, increased metastasis and invasion, likely by a mechanism involving specific increase in translation of many weakly competitive mRNAs encoding proteins known to stimulate cell growth and angiogenesis such as fibroblast growth factor, vascular endothelial growth factor and cyclin D1 (Ruggero et al., 2004; Sonenberg, 2008; Wendel et al., 2004). Moreover, silencing of eIF4e with siRNA duplexes, antisense RNA or by overexpression of the inhibitory 4E-BP 1 results in decreased oncogenic potential of cells (Isabella Bray, 2006). Elevated levels of eIF4e also correlate with a poor prognosis for cancer patients. To promote tumorigenesis, eIF4E must be phosphorylated at Ser 209.

Elevated phosphorylation of eIF4E and increased expression levels of MNK1 and MNK2 have been detected in various solid tumors and lymphomas (Bianchini, Loiarro, & Bielli, 2008; Fan et al., 2009; Hsieh & Ruggero, 2010) and correlate with bad prognosis for patients. Surprisingly, double knock-out mice that lack both mnk1 and mnk2 do not have any apparent phenotype. However the phosphorylation of eIF4E by MNK1 and MNK2 on Ser-209 is critical for the oncogenic activity of eIF4E (Bianchini et al., 2008; Furic et al., 2010; Lim et al., 2013; Topisirovic, Ruiz-Gutierrez, & Borden, 2004; Ueda et al., 2010).

MNK1 and MNK2 are not only implicated in pathways linked to cancer but also in the regulation of immune, autocrine and endocrine responses. Thus, MNK1 and MNK2 regulate cellular response to Escherichia coli lipopolysaccharide (LPS) and Type II Interferon (IFNγ) Signaling (Rowlett et al., 2008). MNK inhibition reduces proinflammatory cytokines known to be important in innate immune responses and inflammation, including TNF, IL-6, IL-10, IL-17 and MCP-1 (Gorentla et al., 2013; Joshi et al., 2011). Thus, there is a link to cytokine-related diseases such as e.g. autoimmune, (auto)inflammatory, neurodegenerative or viral diseases. Further, a link has been established to metabolic diseases (e.g. diabetes, hyperlipidemia and obesity, see e.g. WO 03/037362 and WO 02/103361).

There is the need for compounds which inhibit MNK1 and/or MNK2 kinase activity in a potent manner in order to be in a position to treat diseases linked to their (increased) activity, in particular oncogenic (in particular hematopoietic diseases), autoimmune, inflammatory, metabolic and viral diseases.

### OBJECTS AND SUMMARY OF THE INVENTION

The inventors of the present invention inter alia surprisingly found that a compound of formula (I) as defined herein below (see first aspect) inhibits MNK1 and/or MNK2. Accordingly, a pharmaceutical composition comprising a compound of formula (I) as defined herein below (see second aspect) can be used for the treatment of diseases linked to an increased or aberrant activity of MNK1 and/or MNK2.

In a first aspect, the present invention refers to a compound of formula (I)

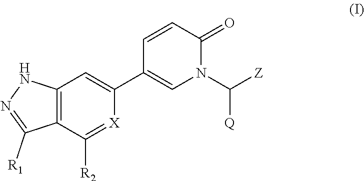

or a salt, stereoisomer, tautomer or N-oxide thereof,
wherein

### X is CR3 or N;

### R1 is

(i) H, halogen, OT¹, N(T²)(T³), NHC(O)T⁴, NHS(O)₂T⁴, C(O)OT¹, C(O)N(T²)(T³), C(O)T⁴, ST¹, S(O)₂T⁴, S(O)₂N(T²)(T³), NO₂, or CN; or
(ii) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iv) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(v) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(vi) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸, S(O)₂N(T⁵)(T⁶), a ring system according to (ii) above, a ring system according to (iii) above, a ring system according to (iv) above and a ring system according to (v) above;
   R2 and R3, if present, are independently
(i) H, halogen, OT¹, N(T²)(T³), NHC(O)T⁴, NHS(O)₂T⁴, C(O)OT¹, C(O)N(T²)(T³), C(O)T⁴, ST¹, S(O)₂T⁴, S(O)₂N(T²)(T³), NO₂, or CN; or
(ii) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) (C₀₋₆alkyl)T¹⁰, (C₃₋₆heteroalkyl)T¹⁰, (C₂₋₆alkenyl)T¹⁰, (C₂₋₆alkynyl)T¹⁰, (CH₂)ₙO(CH₂)ₙT¹⁰, C(O)(CH₂)ₙT¹⁰, C(O)O(CH₂)ₙT¹⁰, C(O)N(T²)[(CH₂)ₙT¹⁰], NHC(O)(CH₂)ₙT¹⁰, N(T²)[(CH₂)ₙT¹⁰)], N(T²)[(CH₂)ₙNHT¹⁰], O(CH₂)ₙNHT¹⁰, (CH₂)ₙN(T²)[(CH₂)ₙT¹⁰], (CH₂)ₙS(CH₂)ₙT¹⁰, S(O)₂(CH₂)ₙT¹⁰, S(O)₂O(CH₂)ₙT¹⁰, S(O)₂N(T²)[(CH₂)ₙT¹⁰], NHS(O)₂(CH₂)ₙT¹⁰ or S(CH₂)ₙNHT¹⁰, wherein n is independently 0, 1, 2, 3 or 4, and wherein T¹⁰ is
   (a) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
   (b) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
   (c) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
   (d) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, oxo, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
   Z is H, halogen, C₁₋₆alkyl or C₃₋₆heteroalkyl, wherein said C₁₋₆alkyl and C₃₋₆heteroalkyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);

### Q is

(i) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from
   (a) C₁₋₆alkyl, C₃₋₆heteroalkyl, C₂₋₆alkenyl and C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(o)OT⁷, C(O)N(T⁵)(T⁶), OC(O)N(T⁵)(T⁶), S(O)₂T⁷, S(O)₂OT⁸ and S(O)₂N(T⁵)(T⁶), and
   (b) halogen, CF₃, OT¹, N(T²)(T³), NHC(O)T⁴, NHC(O)T⁹, NHS(O)₂T⁴, ST¹, S(O)₂T⁴, S(O)₂N(T⁵)(T⁶), NO₂, CN, C(O)H, C(O)OT¹, C(O)N(T²)(T³), C(O)T⁴ and OC(O)N(T²)(T³), and
   (c) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
   (d) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and (e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
   (f) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(ii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iv) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
   T¹, T² and T³ are each independently selected from H, C₁₋₆alkyl and C₃₋₆heteroalkyl, wherein said C₁₋₆alkyl and C₃₋₆heteroalkyl is unsubstituted or independently substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
   T⁴ is C₁₋₆alkyl or C₃₋₆heteroalkyl, wherein said C₁₋₆alkyl and C₃₋₆heteroalkyl is unsubstituted or independently substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁵, S(O)₂OT⁷ and S(O)₂N(T⁵)(T⁶);
   T⁵, T⁶ and T⁷ are each independently selected from H, C₁₋₆alkyl and C₃₋₆heteroalkyl, wherein said C₁₋₆alkyl and C₃₋₆heteroalkyl is unsubstituted or independently substituted with at least one substituent independently selected from halogen, NH₂, NHCH₃, N(CH₃)(CH₃), NO₂, OH, OCH₃, SH, C(O)NH₂, C(O)NHCH₃, C(O)N(CH₃)(CH₃) and CN;
   T⁸ is selected from C₁₋₆alkyl and C₃₋₆heteroalkyl, wherein said C₁₋₆alkyl and C₃₋₆heteroalkyl is unsubstituted or independently substituted with at least one substituent independently selected from halogen, NH₂, NHCH₃, N(CH₃)(CH₃), NO₂, OH, OCH₃, SH, C(O)NH₂, C(O)NHCH₃, C(O)N(CH₃)(CH₃) and CN; and T⁹ is C₂₋₆alkenyl, wherein said C₂₋₆alkenyl is unsubstituted or substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁸, S(O)₂OT⁷ and S(O)₂N(T⁵)(T⁶).

In a preferred embodiment of the first aspect, the present invention refers to a compound of formula (I)

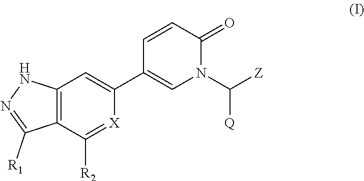

or a salt, stereoisomer, tautomer or N-oxide thereof,
wherein

### X is CR3 or N;

### R1 is

(i) H, halogen, OT¹, N(T²)(T³), NHC(O)T⁴, NHS(O)₂T⁴, C(O)OT¹, C(O)N(T²)(T³), C(O)T⁴, ST¹, S(O)₂T⁴, S(O)₂N(T²)(T³), NO₂, or CN; or
(ii) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iv) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(v) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(vi) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸, S(O)₂N(T⁵)(T⁶), a ring system according to (ii) above, a ring system according to (iii) above, a ring system according to (iv) above and a ring system according to (v) above;
   R2 and R3, if present, are independently
(i) H, halogen, OT¹, N(T²)(T³), NHC(O)T⁴, NHS(O)₂T⁴, C(O)OT¹, C(O)N(T²)(T³), C(O)T⁴, ST¹, S(O)₂T⁴, S(O)₂N(T²)(T³), NO₂, or CN; or
(ii) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) (C₀₋₆alkyl)T¹⁰, (C₃₋₆heteroalkyl)T¹⁰, (C₂₋₆alkenyl)T¹⁰, (C₂₋₆alkynyl)T¹⁰, (CH₂)ₙO(CH₂)ₙT¹⁰, C(O)(CH₂)ₙT¹⁰, C(O)O(CH₂)ₙT¹⁰, C(O)N(T²)[(CH₂)ₙT¹⁰], NHC(O)(CH₂)ₙT¹⁰, N(T²)[(CH₂)ₙT¹⁰)], N(T²)[(CH₂)ₙNHT¹⁰], O(CH₂)ₙNHT¹⁰, (CH₂)ₙN(T²)[(CH₂)ₙT¹⁰], (CH₂)ₙS(CH₂)ₙT¹⁰, S(O)₂(CH₂)ₙT¹⁰, S(O)₂O(CH₂)ₙT¹⁰, S(O)₂N(T²)[(CH₂)ₙT¹⁰], NHS(O)₂(CH₂)ₙT¹⁰ or S(CH₂)ₙNHT¹⁰ wherein n is independently 0, 1, 2, 3 or 4, and wherein T¹⁰ is
   (a) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
   (b) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(o)OT⁷, C(O)N(T⁵)(T⁶), OC(O)N(T⁵)(T⁶), S(O)₂T⁷, S(O)₂OT⁸ and S(O)₂N(T⁵)(T⁶); or
   (c) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
   (d) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, oxo, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT¹ and S(O)₂N(T⁵)(T⁶);
   Z is H, halogen, C₁₋₆alkyl or C₃₋₆heteroalkyl, wherein said C₁₋₆alkyl and C₃₋₆heteroalkyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);

### Q is

(i) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from
   (a) C₁₋₆alkyl, C₃₋₆heteroalkyl, C₂₋₆alkenyl and C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(o)OT⁷, C(O)N(T⁵)(T⁶), OC(O)N(T⁵)(T⁶), S(O)₂T⁷, S(O)₂OT⁸ and S(O)₂N(T⁵)(T⁶), and
   (b) halogen, CF₃, OT¹, N(T²)(T³), NHC(O)T⁴, NHC(O)T⁹, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), and
   (c) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T₆), —OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
   (d) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
   (e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
   (f) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(ii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iv) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
   T¹, T² and T³ are each independently selected from H, C₁₋₆alkyl and C₃₋₆heteroalkyl, wherein said C₁₋₆alkyl and C₃₋₆heteroalkyl is unsubstituted or independently substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
   T⁴ is C₁₋₆alkyl or C₃₋₆heteroalkyl, wherein said C₁₋₆alkyl and C₃₋₆heteroalkyl is unsubstituted or independently substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁸, S(O)₂OT⁷ and S(O)₂N(T⁵)(T⁶); T⁵, T⁶ and T⁷ are each independently selected from H, C₁₋₆alkyl and C₃₋₆heteroalkyl, wherein said C₁₋₆alkyl and C₃₋₆heteroalkyl is unsubstituted or independently substituted with at least one substituent independently selected from halogen, NH₂, NHCH₃, N(CH₃)(CH₃), NO₂, OH, OCH₃, SH, C(O)NH₂, C(O)NHCH₃, C(O)N(CH₃)(CH₃) and CN;
   T⁸ is selected from C₁₋₆alkyl and C₃₋₆heteroalkyl, wherein said C₁₋₆alkyl and C₃₋₆heteroalkyl is unsubstituted or independently substituted with at least one substituent independently selected from halogen, NH₂, NHCH₃, N(CH₃)(CH₃), NO₂, OH, OCH₃, SH, C(O)NH₂, C(O)NHCH₃, C(O)N(CH₃)(CH₃) and CN; and
   T⁹ is C₂₋₆alkenyl, wherein said C₂₋₆alkenyl is unsubstituted or substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁸, S(O)₂OT⁷ and S(O)₂N(T⁵)(T⁶).

The following embodiments relate to R1 as defined above in the first aspect.

In embodiment (1)A, R1 is
(i) as defined for R1 in the first aspect; or
(ii) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iv) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(v) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(vi) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸, and S(O)₂N(T⁵)(T⁶).

In preferred embodiment (1)B, R1 is
(i) as defined for R1 in the first aspect; or
(ii) a monocyclic aromatic carbocyclic ring system with 6 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iii) a monocyclic aromatic heterocyclic ring system with 5 or 6 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iv) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(v) a monocyclic saturated or partially unsaturated non-aromatic heterocyclic ring system with 3, 4, 5, 6 or 7 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(vi) C₁₋₆alkyl, C₂₋₆alkenyl or C₂₋₆alkynyl, wherein said C₁₋₆alkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸, and S(O)₂N(T⁵)(T⁶).

In preferred embodiment (1)C, R1 is
(i) H, halogen, OT¹, N(T²)(T³), NHC(O)T⁴ or
(ii) a monocyclic aromatic carbocyclic ring system with 6 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iii) a monocyclic aromatic heterocyclic ring system with 5 or 6 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(vi) unsubstituted C₁₋₆alkyl, C₂₋₆alkenyl or C₂₋₆alkynyl.

In embodiment (1)D relating to “linear” R1 substituents, R1 is (i) as defined for R1 in the first aspect or (vi) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶).

In a preferred embodiment (1)E relating to “linear” R1 substituents, R1 is (i) as defined for R1 in the first aspect or (vi) C₁₋₆alkyl, wherein said C₁₋₆alkyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶).

In an even more preferred embodiment (1)F relating to “linear” R1 substituents, R1 is (i) H, halogen, OT¹, N(T²)(T³), NHC(O)T⁴ or (vi) unsubstituted C₁₋₆alkyl.

In another also particularly preferred embodiment (1)G relating to “linear” R1 substituents, R1 is H, halogen, OH, NH₂, NHC(O)CH₃ or CH₃.

In embodiment (1)H relating to “cyclic” R1 substituents, R1 is (ii) as defined for R1 in the first aspect or (iii) as defined for R1 in the first aspect or (iv) as defined for R1 in the first aspect or (v) as defined for R1 in the first aspect.

In preferred embodiment (1)I relating to “cyclic” R1 substituents, R1 is (ii) as defined in embodiment (1)B or (iii) as defined in embodiment (1)B or (iv) as defined in embodiment (1)B or (v) as defined in embodiment (1)B.

In preferred embodiment (1)J relating to “cyclic” R1 substituents, R1 is (ii) as defined in embodiment (1)C or (iii) as defined in embodiment (1)C.

In the most preferred embodiment (1)K, R1 is H or NH₂, preferably NH₂.

The following embodiments relate to X as defined above in the first aspect.

In embodiment (2)A, X is N. In the preferred embodiment (2)B, X is CR3.

The following embodiments relate to R2 and R3, if present, as defined above in the first aspect. It is clear from the definition of X in the first aspect that R3 is only present if X is CR3, as defined in embodiment (2)B. If reference is in the following made to R2 and R3, it is understood by the skilled person that this relates to a situation, where (i) both, R2 and R3 are present due to the definition of X being CR3, and where (ii) R3 is absent due to the definition of X being N. In the latter case, the embodiments of course then only apply for R2.

In embodiment (3)A, R2 and R3, if present, are independently
(i) as defined in the first aspect for R2 and R3, if present; or
(ii) as defined in the first aspect for R2 and R3, if present; or
(iii) (C₀₋₆alkyl)T¹⁰, (C₃₋₆heteroalkyl)T¹⁰, (C₂₋₆alkenyl)T¹⁰, (C₂₋₆alkynyl)T¹⁰, (CH₂)ₙO(CH₂)ₙT¹⁰, C(O)(CH₂)ₙT¹⁰, C(O)O(CH₂)ₙT¹⁰, C(O)N(T²)[(CH₂)ₙT¹⁰], NHC(O)(CH₂)ₙT¹⁰, N(T²)[(CH₂)ₙT¹⁰)], N(T²)[(CH₂)ₙNHT¹⁰], O(CH₂)ₙNHT¹⁰, (CH₂)ₙN(T²)[(CH₂)ₙT¹⁰], (CH₂)ₙS(CH₂)ₙT¹⁰, S(O)₂(CH₂)ₙT¹⁰, S(O)₂O(CH₂)ₙT¹⁰, S(O)₂N(T²)[(CH₂)ₙT¹⁰], NHS(O)₂(CH₂)ₙT¹⁰ or S(CH₂)ₙNHT¹⁰, wherein n is independently 0, 1, 2, 3 or 4, and wherein T¹⁰ is
   a) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(o)OT¹, C(O)N(T²)(T³), C(O)T⁴ and OC(O)N(T²)(T³); or
   b) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(o)OT¹, C(O)N(T²)(T³), C(O)T⁴ and OC(O)N(T²)(T³); or
   c) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
   d) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, oxo, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

In embodiment (3)B, R2 and R3, if present, are independently
(i) as defined in the first aspect for R2 and R3, if present; or
(ii) as defined in the first aspect for R2 and R3, if present; or
(iii) (C₀₋₆alkyl)T¹⁰, (C₃₋₆heteroalkyl)T¹⁰, (C₂₋₆alkenyl)T¹⁰, (C₂₋₆alkynyl)T¹⁰, (CH₂)ₙO(CH₂)ₙT¹⁰, C(O)(CH₂)ₙT¹⁰, C(O)O(CH₂)ₙT¹⁰C(O)N(T²)[(CH₂)ₙT¹⁰], NHC(O)(CH₂)ₙT¹⁰, N(T²)[(CH₂)ₙT¹⁰)], N(T²)[(CH₂)ₙNHT¹⁰], O(CH₂)ₙNHT¹⁰, (CH₂)ₙN(T²)[(CH₂)ₙT¹⁰], (CH₂)ₙS(CH₂)ₙT¹⁰, S(O)₂(CH₂)ₙT¹⁰, S(O)₂O(CH₂)ₙT¹⁰, S(O)₂N(T²)[(CH₂)ₙT¹⁰], NHS(O)₂(CH₂)ₙT¹⁰ or S(CH₂)ₙNHT¹⁰ wherein n is independently 0, 1 or 2, and wherein T¹⁰ is
   a) a monocyclic aromatic carbocyclic ring system with 6 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
   b) a monocyclic aromatic heterocyclic ring system with 5 or 6 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
   c) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
   d) a monocyclic saturated or partially unsaturated non-aromatic heterocyclic ring system with 3, 4, 5, 6 or 7 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, oxo, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

In embodiment (3)C, R2 and R3, if present, are independently
(i) as defined in the first aspect for R2 and R3, if present; or
(ii) as defined in the first aspect for R2 and R3, if present.

In embodiment (3)D, R2 and R3, if present, are independently H, halogen, OH, NH₂, NO₂ or unsubstituted C₁₋₆alkyl.

In preferred embodiment (3)E, R2 is H and R3, if present, is
(i) as defined in the first aspect for R3; or
(ii) as defined in the first aspect for R3.

In preferred embodiment (3)F, R2 is H and R3, if present, is halogen, OH, NH₂, NO₂ or unsubstituted C₁₋₆alkyl.

In preferred embodiment (3)G, R3, if present, is H and R2 is
(i) as defined in the first aspect for R2; or
(ii) as defined in the first aspect for R2.

In preferred embodiment (3)H, R3, if present, is H and R2 is halogen, OH, NH₂, NO₂ or unsubstituted C₁₋₆alkyl.

In the preferred embodiment (3)1, R2 and R3, if present, are both H.

In preferred embodiment (3)J, R3, if present, is H and R2 is (iii) as defined in the first aspect for R2. In preferred embodiment (3)K, R3, if present, is H and R2 is (iii) as defined in embodiment (3)A. In preferred embodiment (3)L, R3, if present, is H and R2 is (iii) as defined in embodiment (3)B.

The following embodiments relate to Z as defined above in the first aspect.

In embodiment (4)A, Z is H, halogen or C₁₋₆alkyl, wherein said C₁₋₆alkyl is unsubstituted or substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶).

In preferred embodiment (4)B, Z is H or CH₃, preferably H.

The following embodiments relate to Q as defined above in the first aspect.

In embodiment (5)A, Q is
(i) as defined in the first aspect for Q with an optional at least one substituent selected from (a), (b) and (c) as defined under (i) in the first aspect for Q; or
(ii) as defined in the first aspect for Q; or
(iii) as defined in the first aspect for Q; or
(iv) as defined in the first aspect for Q.

In embodiment (5)B, Q is
(i) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from
   (a) C₁₋₆alkyl, C₂₋₆alkenyl and C₂₋₆alkynyl, wherein said C₁₋₆alkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶), and
   (b) halogen, CF₃, OT¹, N(T²)(T³), NHC(O)T⁴, NHC(O)T⁹, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), and
   (c) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); and
   (d) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); and
   (e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); and
   (f) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(ii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iii) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iv) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(o)OT¹, C(O)N(T²)(T³), C(O)T⁴ and OC(O)N(T²)(T³).

In preferred embodiment (5)B 1 thereof, Q is (i) as defined in embodiment (5)B with an optional at least one substituent selected from (a), (b) and (c) as defined under (i) in embodiment (5)B; or (ii) as defined in embodiment (5)B; or (iii) as defined in embodiment (5)B; or (iv) as defined in embodiment 5(B).

In still another embodiment (5)C, Q is (ii) as defined in the first aspect for Q. In a preferred embodiment (5)C1 thereof, Q is a monocyclic aromatic heterocyclic ring system with 5 or 6 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

In preferred embodiment (5)D, Q has the following structure

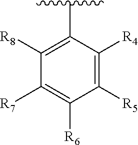

wherein R₄, R₅, R₆, R₇ and R₈ are independently
(a) C₁₋₆alkyl, C₂₋₆alkenyl or C₂₋₆alkynyl, wherein said C₁₋₆alkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(b) H, halogen, CF₃, OT¹, N(T²)(T³), NHC(O)T⁴, NHC(O)T⁹, NHS(O)₂T⁴, ST¹, S(O)₂T⁴, NO₂, CN, C(O)H, C(O)OT¹, C(O)N(T²)(T³), C(O)T⁴ or OC(O)N(T²)(T³); or
(c) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(d) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(f) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

In embodiment (5)E, Q has the following structure

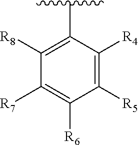

wherein R₄, R₅, R₆, R₇ and R₈ are independently (a) as defined in embodiment (5)D, or (b) as defined in embodiment (5)D, or (c) as defined in embodiment (5)D.

In embodiment (5)F, Q has the following structure

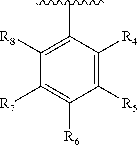

wherein R₄, R₅, R₆, R₇ and R₈ are independently (b) as defined in embodiment (5)D, or (c) as defined in embodiment (5)D.

In embodiment (5)G, Q has the following structure

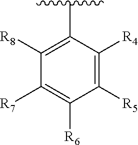

wherein two of the substituents selected from the group consisting of R₄, R₅, R₆, R₇ and R₈ are independently (a) as defined in embodiment (5)D, or (b) as defined in embodiment (5)D, or (c) as defined in embodiment (5)D, or (d) as defined in embodiment (5)D, or (e) as defined in embodiment (5)D, or (f) as defined in embodiment (5)D, and the remaining three substituents of the above group are H. It is preferred that said two substituents are R₄ and R₅.

In embodiment (5)H, Q has the following structure

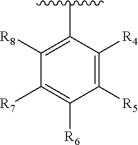

wherein two of the substituents selected from the group consisting of R₄, R₅, R₆, R₇ and R₈ are independently (a) as defined in embodiment (5)D, or (b) as defined in embodiment (5)D, or (c) as defined in embodiment (5)D, and the remaining three substituents of the above group are H. It is preferred that said two substituents are R₄ and R₅.

In embodiment (5)I, Q has the following structure

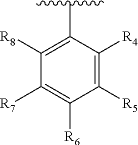

wherein two of the substituents selected from the group consisting of R₄, R₅, R₆, R₇ and R₈ are independently (b) as defined in embodiment (5)D, or (c) as defined in embodiment (5)D, and the remaining three substituents of the above group are H. It is preferred that said two substituents are R₄ and R₅.

In embodiment (5)J, Q has the following structure

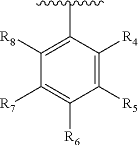

wherein one of the substituents selected from the group consisting of R₄, R₅, R₆, R₇ and R₈ is (a) as defined in embodiment (5)D, or (b) as defined in embodiment (5)D, or (c) as defined in embodiment (5)D, or (d) as defined in embodiment (5)D, or (e) as defined in embodiment (5)D, or (f) as defined in embodiment (5)D, and the remaining four substituents of the above group are H. It is preferred that said substituent is R₄ or R₅. It can be preferred that said substituent is R₅.

In embodiment (5)K, Q has the following structure

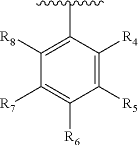

wherein one of the substituents selected from the group consisting of R₄, R₅, R₆, R₇ and R₈ is (a) as defined in embodiment (5)D, or (b) as defined in embodiment (5)D, or (c) as defined in embodiment (5)D, and the remaining four substituents of the above group are H. It is preferred that said substituent is R₄ or R₅. It can be preferred that said substituent is R₅.

In embodiment (5)L, Q has the following structure

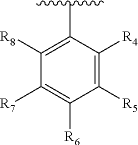

wherein one of the substituents selected from the group consisting of R₄, R₅, R₆, R₇ and R₈ is (b) as defined in embodiment (5)D, or (c) as defined in embodiment (5)D, and the remaining four substituents of the above group are H. It is preferred that said substituent is R₄ or R₅. It can be preferred that said substituent is R₅.

In preferred embodiment (5)M, Q has the following structure

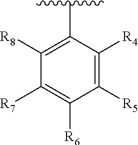

wherein R₄, R₅, R₆, R₇ and R₈ are independently
(a) C₁₋₆alkyl, C₂₋₆alkenyl or C₂₋₆alkynyl, wherein said C₁₋₆alkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(b) H, halogen, CF₃, OT¹, N(T²)(T³), NHC(O)T⁴, NHC(O)T⁹, NHS(O)₂T⁴, ST¹, S(O)₂T⁴, NO₂, CN, C(O)H, C(O)OT¹, C(O)N(T²)(T³), C(O)T⁴ or OC(O)N(T²)(T³); or
(c) a monocyclic aromatic heterocyclic ring system with 5 or 6 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(d) a monocyclic aromatic carbocyclic ring system with 6 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(f) a monocyclic saturated or partially unsaturated non-aromatic heterocyclic ring system with 3, 4, 5, 6 or 7 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

In embodiment (5)N, Q has the following structure

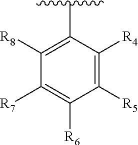

wherein R₄, R₅, R₆, R₇ and R₈ are independently (a) as defined in embodiment (5)M, or (b) as defined in embodiment (5)M, or (c) as defined in embodiment (5)M.

In embodiment (5)0, Q has the following structure

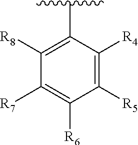

wherein R₄, R₅, R₆, R₇ and R₈ are independently (b) as defined in embodiment (5)M, or (c) as defined in embodiment (5)M.

In embodiment (5)P, Q has the following structure

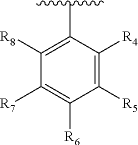

wherein two of the substituents selected from the group consisting of R₄, R₅, R₆, R₇ and R₈ are independently (a) as defined in embodiment (5)M, or (b) as defined in embodiment (5)M, or (c) as defined in embodiment (5)M, or (d) as defined in embodiment (5)M, or (e) as defined in embodiment (5)M, or (f) as defined in embodiment (5)M, and the remaining three substituents of the above group are H. It is preferred that said two substituents are R₄ and R₅.

In embodiment (5)Q, Q has the following structure

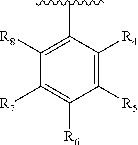

wherein two of the substituents selected from the group consisting of R₄, R₅, R₆, R₇ and R₈ are independently (a) as defined in embodiment (5)M, or (b) as defined in embodiment (5)M, or (c) as defined in embodiment (5)M, and the remaining three substituents of the above group are H. It is preferred that said two substituents are R₄ and R₅.

In embodiment (5)R, Q has the following structure

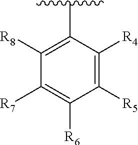

wherein two of the substituents selected from the group consisting of R₄, R₅, R₆, R₇ and R₈ are independently (b) as defined in embodiment (5)M, or (c) as defined in embodiment (5)M, and the remaining three substituents of the above group are H. It is preferred that said two substituents are R₄ and R₅.

In embodiment (5)S, Q has the following structure

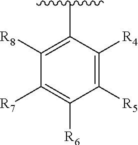

wherein one of the substituents selected from the group consisting of R₄, R₅, R₆, R₇ and R₈ is (a) as defined in embodiment (5)M, or (b) as defined in embodiment (5)M, or (c) as defined in embodiment (5)M, or (d) as defined in embodiment (5)M, or (e) as defined in embodiment (5)M, or (f) as defined in embodiment (5)M, and the remaining four substituents of the above group are H. It is preferred that said substituent is R₄ or R₅. It can be preferred that said substituent is R₅.

In embodiment (5)T, Q has the following structure

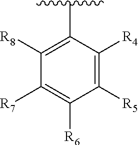

wherein one of the substituents selected from the group consisting of R₄, R₅, R₆, R₇ and R₈ is (a) as defined in embodiment (5)M, or (b) as defined in embodiment (5)M, or (c) as defined in embodiment (5)M, and the remaining four substituents of the above group are H. It is preferred that said substituent is R₄ or R₅. It can be preferred that said substituent is R₅.

In embodiment (5)U, Q has the following structure

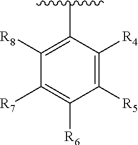

wherein one of the substituents selected from the group consisting of R₄, R₅, R₆, R₇ and R₈ is (b) as defined in embodiment (5)M, or (c) as defined in embodiment (5)M, and the remaining four substituents of the above group are H. It is preferred that said substituent is R₄ or R₅. It can be preferred that said substituent is R₅.

In preferred embodiment (5)V, Q has the following structure

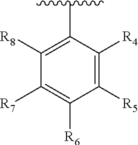

wherein R₄, R₅, R₆, R₇ and R₈ are all H.

If any of the above embodiments are combined, it is preferred to select embodiment (2)B to be combined with others and/or embodiment (3)I to be combined with others and/or embodiment (4)B to be combined with others. Likewise, it is preferred to combine embodiment (1)C with others and/or embodiment (5)M with others. Any of embodiments (5)S to (5)U may also be preferably combined with others.

The following combinations of embodiments as described above are preferred (if no specific embodiment for a substituent is given, the definition of the first aspect of course applies):
embodiment (1)K+any of embodiments (5)A to (5)V;
embodiment (1)K+any of embodiments (5)B to (5)C1;
embodiment (1)K+any of embodiments (5)M to (5)V;
embodiment (3)D+any of embodiments (5)A to (5)V;
embodiment (3)D+any of embodiments (5)B to (5)C1;
embodiment (3)D+any of embodiments (5)M to (5)V;
embodiment (1)C+embodiment (2)B+embodiment (3)C+embodiment (4)B+any of embodiments (5)A to (5)V;
embodiment (1)C+embodiment (2)B+embodiment (3)D+embodiment (4)B+any of embodiments (5)A to (5)V;
embodiment (1)C+embodiment (2)B+embodiment (3)I+embodiment (4)B+any of embodiments (5)A to (5)V;
embodiment (1)K+embodiment (2)B+embodiment (3)C+embodiment (4)B+any of embodiments (5)A to (5)V;
embodiment (1)K+embodiment (2)B+embodiment (3)D+embodiment (4)B+any of embodiments (5)A to (5)V;
embodiment (1)K+embodiment (2)B+embodiment (3)I+embodiment (4)B+any of embodiments (5)A to (5)V;
embodiment (1)C+embodiment (2)B+embodiment (3)C+embodiment (4)B+any of embodiments (5)M to (5)V;
embodiment (1)C+embodiment (2)B+embodiment (3)D+embodiment (4)B+any of embodiments (5)M to (5)V;
embodiment (1)C+embodiment (2)B+embodiment (3)I+embodiment (4)B+any of embodiments (5)M to (5)V;
embodiment (1)K+embodiment (2)B+embodiment (3)C+embodiment (4)B+any of embodiments (5)M to (5)V;
embodiment (1)K+embodiment (2)B+embodiment (3)D+embodiment (4)B+any of embodiments (5)M to (5)V;
embodiment (1)K+embodiment (2)B+embodiment (3)I+embodiment (4)B+any of embodiments (5)M to (5)V;

In a preferred embodiment, the compound of the present invention is selected form the group consisting of 1-benzyl-5-(1H-indazol-6-yl)-1,2-dihydropyridin-2-one; 1-(2-fluorobenzyl)-5-(1H-indazol-6-yl)pyridin-2(1H)-one; 2-{[5-(1H-indazol-6-yl)-2-oxopyridin-1 (2H)-yl]methyl}benzonitrile; 1-(1,3-benzodioxol-5-ylmethyl)-5-(1H-indazol-6-yl)pyridin-2(1H)-one; 1-(2-chlorobenzyl)-5-(1H-indazol-6-yl)pyridin-2(1H)-one; 5-(1H-indazol-6-yl)-1-(4-methoxybenzyl)pyridin-2(1H)-one; 1-(3,4-dichlorobenzyl)-5-(1H-indazol-6-yl)pyridin-2(1H)-one; 5-(1H-indazol-6-yl)-1-(3-methoxybenzyl)pyridin-2(1H)-one; 5-(1H-indazol-6-yl)-1-(3-nitrobenzyl)pyridin-2(1H)-one; 1-(4-fluoro-3-nitrobenzyl)-5-(1H-indazol-6-yl)pyridin-2(1H)-one; 5-(1H-indazol-6-yl)-1-(pyridin-2-ylmethyl)pyridin-2(1H)-one; 1-(3-chlorobenzyl)-5-(1H-indazol-6-yl)pyridin-2(1H)-one; 5-(1H-indazol-6-yl)-1-[(2-methyl-1,3-thiazol-5-yl)methyl]pyridin-2(1H)-one; 5-(1H-indazol-6-yl)-1-[4-(trifluoromethyl)benzyl]pyridin-2(1H)-one; methyl 3-{[5-(1H-indazol-6-yl)-2-oxopyridin-1 (2H)-yl]methyl}benzoate; methyl 2-{[5-(1H-indazol-6-yl)-2-oxopyridin-1 (2H)-yl]methyl}benzoate; 5-(1H-indazol-6-yl)-1-(pyridin-4-ylmethyl)pyridin-2(1H)-one; 5-(1H-indazol-6-yl)-1-[2-(trifluoromethyl)benzyl]pyridin-2(1H)-one; 5-(1H-indazol-6-yl)-1-[3-(trifluoromethyl)benzyl]pyridin-2(1H)-one; 3-{[5-(1H-indazol-6-yl)-2-oxopyridin-1(2H)-yl]methyl}benzonitrile; 3-{[5-(1H-indazol-6-yl)-2-oxopyridin-1(2H)-yl]methyl}benzamide; 5-(1H-indazol-6-yl)-1-(1-phenylethyl)pyridin-2(1H)-one; 5-(1H-indazol-6-yl)-1-[2-(trifluoromethoxy)benzyl]pyridin-2(1H)-one; 5-(1H-indazol-6-yl)-1-[2-(thiophen-2-yl)benzyl]pyridin-2(1H)-one; 1-[3-(difluoromethoxy)benzyl]-5-(1H-indazol-6-yl)pyridin-2(1H)-one; 1-[(5-chlorothiophen-2-yl)methyl]-5-(1H-indazol-6-yl)pyridin-2(1H)-one; 5-(1H-indazol-6-yl)-1-[2-(pyridin-4-yl)benzyl]pyridin-2(1H)-one; 5-(1H-indazol-6-yl)-1-[2-(pyridin-3-yl)benzyl]pyridin-2(1H)-one; 5-(1H-indazol-6-yl)-1-(1H-pyrazol-4-ylmethyl)pyridin-2(1H)-one; 5-(1H-indazol-6-yl)-1-[3-(thiophen-2-yl)benzyl]pyridin-2(1H)-one; 5-(1H-indazol-6-yl)-1-[3-(pyridin-3-yl)benzyl]pyridin-2(1H)-one; 4-{[5-(1H-indazol-6-yl)-2-oxopyridin-1 (2H)-yl]methyl}benzonitrile; 1-[3-(hydroxymethyl)benzyl]-5-(1H-indazol-6-yl)pyridin-2(1H)-one; 1-[(4-bromothiophen-2-yl)methyl]-5-(1H-indazol-6-yl)pyridin-2(1H)-one; 5-(1H-indazol-6-yl)-1-(thiophen-3-ylmethyl)pyridin-2(1H)-one; 1-[2-(hydroxymethyl)benzyl]-5-(1H-indazol-6-yl)pyridin-2(1H)-one; 1-[2-(furan-3-yl)benzyl]-5-(1H-indazol-6-yl)pyridin-2(1H)-one; 5-(1H-indazol-6-yl)-1-[2-(thiophen-3-yl)benzyl]pyridin-2(1H)-one; 1-[(2-chloropyridin-4-yl)methyl]-5-(1H-indazol-6-yl)pyridin-2(1H)-one; 5-(1H-indazol-6-yl)-1-[2-(1H-pyrazol-3-yl)benzyl]pyridin-2(1H)-one; 1-(3-chloro-4-fluorobenzyl)-5-(1H-indazol-6-yl)pyridin-2(1H)-one; 1-(3-chlorobenzyl)-5-(1H-indazol-6-yl)pyridin-2(1H)-one; 1-(2-fluoro-3-nitrobenzyl)-5-(1H-indazol-6-yl)pyridin-2(1H)-one; 1-(5-fluoro-2-nitrobenzyl)-5-(1H-indazol-6-yl)pyridin-2(1H)-one; 1-(3-fluoro-2-nitrobenzyl)-5-(1H-indazol-6-yl)pyridin-2(1H)-one; 1-[5-chloro-2-(thiophen-3-yl)benzyl]-5-(1H-indazol-6-yl)pyridin-2(1H)-one; 1-(2-ethenylbenzyl)-5-(1H-indazol-6-yl)pyridin-2(1H)-one; 5-(1H-indazol-6-yl)-1-(tetrahydro-2H-pyran-4-ylmethyl)pyridin-2(1H)-one; 5-(3-amino-1H-indazol-6-yl)-1-(2-fluorobenzyl)pyridin-2(1H)-one; 1-benzyl-5-(3-bromo-1H-indazol-6-yl)pyridin-2(1H)-one; 5-(1H-indazol-6-yl)-1-(2-nitrobenzyl)pyridin-2(1H)-one; 1-(cyclopropylmethyl)-5-(1H-indazol-6-yl)pyridin-2(1H)-one; 5-(1H-indazol-6-yl)-1-(pyridin-3-ylmethyl)pyridin-2(1H)-one; 1-(cyclohexylmethyl)-5-(1H-indazol-6-yl)pyridin-2(1H)-one; 1-benzyl-5-(4-nitro-1H-indazol-6-yl)pyridin-2(1H)-one; 5-(3-amino-1H-indazol-6-yl)-1-benzylpyridin-2(1H)-one; 5-(3-amino-1H-indazol-6-yl)-1-(3-chlorobenzyl)pyridin-2(1H)-one; 5-(3-amino-1H-indazol-6-yl)-1-[2-(trifluoromethyl)benzyl]pyridin-2(1H)-one; 5-(3-amino-1H-indazol-6-yl)-1-[3-(trifluoromethyl)benzyl]pyridin-2(1H)-one; 5-(3-amino-1H-indazol-6-yl)-1-(1-phenylethyl)pyridin-2(1H)-one; 5-(3-amino-5-methyl-1H-indazol-6-yl)-1-benzylpyridin-2(1H)-one; 1-benzyl-5-(1H-pyrazolo[4,3-c]pyridin-6-yl)pyridin-2(1H)-one; 1-benzyl-5-(3-hydroxy-1H-indazol-6-yl)pyridin-2(1H)-one; N-[6-(1-benzyl-6-oxo-1,6-dihydropyridin-3-yl)-1H-indazol-3-yl]acetamide; 1-benzyl-5-(3-methyl-1H-indazol-6-yl)pyridin-2(1H)-one; 5-(3-amino-1H-indazol-6-yl)-1-[(5-chlorothiophen-2-yl)methyl]pyridin-2(1H)-one; 1-benzyl-5-(5-nitro-1H-indazol-6-yl)pyridin-2(1H)-one; 5-(3-amino-1H-indazol-6-yl)-1-(thiophen-3-ylmethyl)pyridin-2(1H)-one; 5-(3-amino-1H-indazol-6-yl)-1-[2-(thiophen-2-yl)benzyl]pyridin-2(1H)-one; N-(3-{[5-(3-amino-1H-indazol-6-yl)-2-oxopyridin-1 (2H)-yl]methyl}phenyl)acetamide; 5-(3-amino-1H-indazol-6-yl)-1-(3-fluorobenzyl)pyridin-2(1H)-one; 5-(3-amino-1H-indazol-6-yl)-1-(2-fluoro-3-nitrobenzyl)pyridin-2(1H)-one; 5-(3-amino-1H-indazol-6-yl)-1-(5-fluoro-2-nitrobenzyl)pyridin-2(1H)-one; 5-(3-amino-1H-indazol-6-yl)-1-(3-chloro-4-fluorobenzyl)pyridin-2(1H)-one; 5-(3-amino-1H-indazol-6-yl)-1-(3-fluoro-2-nitrobenzyl)pyridin-2(1H)-one; 5-(3-amino-1H-indazol-6-yl)-1-[5-chloro-2-(thiophen-3-yl)benzyl]pyridin-2(1H)-one; 5-(3-amino-1H-indazol-6-yl)-1-(2-ethenylbenzyl)pyridin-2(1H)-one; 5-(3-amino-1H-indazol-6-yl)-1-(2-ethenylbenzyl)pyridin-2(1H)-one; 5-(4-amino-1H-indazol-6-yl)-1-benzylpyridin-2(1H)-one; 1-benzyl-5-[3-(pyridin-4-yl)-1H-indazol-6-yl]pyridin-2(1H)-one; 1-benzyl-5-[3-(4-hydroxyphenyl)-1H-indazol-6-yl]pyridin-2(1H)-one; 1-benzyl-5-[3-(3-methoxyphenyl)-1H-indazol-6-yl]-1,2-dihydropyridin-2-one; 1-benzyl-5-[3-(3-methylphenyl)-1H-indazol-6-yl]-1,2-dihydropyridin-2-one; N-(3-{[5-(3-amino-1H-indazol-6-yl)-2-oxo-1,2-dihydropyridin-1-yl]methyl}phenyl)-2-fluoroacetamide; N-(3-{[5-(3-amino-1H-indazol-6-yl)-2-oxo-1,2-dihydropyridin-1-yl]methyl}-4-fluorophenyl)acetamide; N-(2-{[5-(3-amino-1H-indazol-6-yl)-2-oxo-1,2-dihydropyridin-1-yl]methyl}phenyl)acetamide; N-(3-{[5-(3-amino-1H-indazol-6-yl)-2-oxo-1,2-dihydropyridin-1-yl]methyl}-2-fluorophenyl)acetamide; 2-{[5-(3-amino-1H-indazol-6-yl)-2-oxo-1,2-dihydropyridin-1-yl]methyl}benzene-1-sulfonamide; 1-[2-(bromomethyl)benzyl]-5-(1H-indazol-6-yl)pyridin-2(1H)-one; 1-[3-(bromomethyl)benzyl]-5-(1H-indazol-6-yl)pyridin-2(1H)-one; 1-(3-hydroxybenzyl)-5-(1H-indazol-6-yl)pyridin-2(1H)-one; 5-(3-amino-1H-indazol-6-yl)-1-(3-hydroxybenzyl)pyridin-2(1H)-one; 2-{[5-(1H-indazol-6-yl)-2-oxopyridin-1(2H)-yl]methyl}benzamide; 1-{4-[(2-aminoethyl)amino]-3-nitrobenzyl}-5-(1H-indazol-6-yl)pyridin-2(1H)-one hydrochloride; 1-(3-aminobenzyl)-5-(1H-indazol-6-yl)pyridin-2(1H)-one hydrochloride; 1-[(6-aminopyridin-2-yl)methyl]-5-(1H-indazol-6-yl)pyridin-2(1H)-one hydrochloride; 3-amino-N-(3-{[5-(1H-indazol-6-yl)-2-oxopyridin-1(2H)-yl]methyl}phenyl)propanamide hydrochloride; 5-[3-amino-4-({[(1R,4R)-4-aminocyclohexyl]methyl}amino)-1H-indazol-6-yl]-1-[(3-chlorophenyl)methyl]-1,2-dihydropyridin-2-one hydrochloride; tert-butyl 4-{[(3-amino-6-{1-[(3-chlorophenyl)methyl]-6-oxo-1,6-dihydropyridin-3-yl}-1H-indazol-4-yl)amino]methyl}piperidine-1-carboxylate hydrochloride; 5-[3-amino-4-(piperidin-4-ylmethoxy)-1H-indazol-6-yl]-1-[(3-chlorophenyl)methyl]-1,2-dihydropyridin-2-one hydrochloride; 5-{3-amino-4-[(pyrrolidin-3-ylmethyl)amino]-1H-indazol-6-yl}-1-[(3-chlorophenyl)methyl]-1,2-dihydropyridin-2-one hydrochloride; 5-{3-amino-4-[(piperidin-4-yl)amino]-1H-indazol-6-yl}-1-[(3-chlorophenyl)methyl]-1,2-dihydropyridin-2-one hydrochloride; N-(3-{[5-(1H-indazol-6-yl)-2-oxopyridin-1 (2H)-yl]methyl}phenyl)acetamide; N-(3-{[5-(1H-indazol-6-yl)-2-oxopyridin-1(2H)-yl]methyl}phenyl)prop-2-enamide; (2Z)-4-(dimethylamino)-N-(2-{[5-(1H-indazol-6-yl)-2-oxo-1,2-dihydropyridin-1-yl]methyl}phenyl)but-2-enamide; 1-(2-aminobenzyl)-5-(1H-indazol-6-yl)pyridin-2(1H)-one; 5-(5-amino-1H-indazol-6-yl)-1-benzylpyridin-2(1H)-one; 5-{3-amino-4-[(oxan-4-ylmethyl)amino]-1H-indazol-6-yl}-1-[(3-chlorophenyl)methyl]-1,2-dihydropyridin-2-one; 5-{3-amino-4-[(oxolan-3-ylmethyl)amino]-1H-indazol-6-yl}-1-[(3-chlorophenyl)methyl]-1,2-dihydropyridin-2-one; 5-(4-{[(1-acetylpiperidin-4-yl)methyl]amino}-3-amino-1H-indazol-6-yl)-1-[(3-chlorophenyl)methyl]-1,2-dihydropyridin-2-one; N-[3-({5-[3-amino-4-(oxan-4-ylmethoxy)-1H-indazol-6-yl]-2-oxo-1,2-dihydropyridin-1-yl}methyl)phenyl]acetamide; 5-(4-{[(1-acetylpiperidin-3-yl)methyl]amino}-3-amino-1H-indazol-6-yl)-1-[(3-chlorophenyl)methyl]-1,2-dihydropyridin-2-one; 5-[3-amino-4-(oxan-4-ylmethoxy)-1H-indazol-6-yl]-1-[(3-chlorophenyl)methyl]-1,2-dihydropyridin-2-one.

In another preferred embodiment, the compound of the present invention is selected from the group consisting of 5-(3-amino-1H-indazol-6-yl)-1-benzylpyridin-2(1H)-one; 5-(3-amino-1H-indazol-6-yl)-1-(3-chlorobenzyl)pyridin-2(1H)-one; 1-(3-hydroxybenzyl)-5-(1H-indazol-6-yl)pyridin-2(1H)-one; 5-(3-amino-1H-indazol-6-yl)-1-(3-hydroxybenzyl)pyridin-2(1H)-one; 5-bromo-1-[(5-chlorothiophen-2-yl)methyl]pyridin-2(1H)-one; 1-[(3-fluoro-2-nitrophenyl)methyl]-5-(tetramethyl-1,3,2-dioxaborolan-2-yl)-1,2-dihydropyridin-2-one; 1-(naphthalen-2-ylmethyl)-5-(tetramethyl-1,3,2-dioxaborolan-2-yl)-1,2-dihydropyridin-2-one; 1-(3-chloro-4-fluorobenzyl)-5-(1H-indazol-6-yl)pyridin-2(1H)-one; N-(3-{[5-(3-amino-1H-indazol-6-yl)-2-oxopyridin-1(2H)-yl]methyl}phenyl)acetamide; 5-(3-amino-1H-indazol-6-yl)-1-(2-fluorobenzyl)pyridin-2(1H)-one; 5-bromo-1-(naphthalen-2-ylmethyl)pyridin-2(1H)-one; 5-bromo-1-[2-(1H-pyrazol-3-yl)benzyl]pyridin-2(1H)-one; 5-bromo-1-(3-chloro-4-fluorobenzyl)pyridin-2(1H)-one; 5-(3-amino-1H-indazol-6-yl)-1-(2-fluorobenzyl)pyridin-2(1H)-one; 1-(5-fluoro-2-nitrobenzyl)-5-(1H-indazol-6-yl)pyridin-2(1H)-one; 5-(3-amino-1H-indazol-6-yl)-1-(2-fluoro-3-nitrobenzyl)pyridin-2(1H)-one; 5-(3-amino-1H-indazol-6-yl)-1-(5-fluoro-2-nitrobenzyl)pyridin-2(1H)-one; 5-(3-amino-1H-indazol-6-yl)-1-(3-chloro-4-fluorobenzyl)pyridin-2(1H)-one; 5-(3-amino-1H-indazol-6-yl)-1-[5-chloro-2-(thiophen-3-yl)benzyl]pyridin-2(1H)-one; 5-[3-amino-4-({[(1r,4r)-4-aminocyclohexyl]methyl}amino)-1H-indazol-6-yl]-1-[(3-chlorophenyl)methyl]-1,2-dihydropyridin-2-one; tert-butyl 4-{[(3-amino-6-{1-[(3-chlorophenyl)methyl]-6-oxo-1,6-dihydropyridin-3-yl}-1H-indazol-4-yl)amino]methyl}piperidine-1-carboxylate; 5-[3-amino-4-(piperidin-4-ylmethoxy)-1H-indazol-6-yl]-1-[(3-chlorophenyl)methyl]-1,2-dihydropyridin-2-one; 5-{3-amino-4-[(oxan-4-ylmethyl)amino]-1H-indazol-6-yl}-1-[(3-chlorophenyl)methyl]-1,2-dihydropyridin-2-one; and 5-{3-amino-4-[(oxolan-3-ylmethyl)amino]-1H-indazol-6-yl}-1-[(3-chlorophenyl)methyl]-1,2-dihydropyridin-2-one.

In another embodiment, the salt referred to in the first aspect is a pharmaceutically acceptable salt selected from the group consisting of the hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate. The hydrochloride salt can be particularly preferred.

In a second aspect, the present invention is concerned with a pharmaceutical composition comprising the compound according to the first aspect as outlined above, including all embodiments and combinations of embodiments as mentioned above. Put in different words, this aspect can be formulated as the compound according to the first aspect as outlined above, including all embodiments and combinations of embodiments as mentioned above, for use as medicament. Embodiments of the second aspect are referred to when describing the present invention in more detail below.

In a third aspect, the present invention is concerned with a pharmaceutical composition according to the second aspect of the present invention for use in the treatment of specific diseases, particularly in the treatment of cancer, an autoimmune disease and an inflammatory disease as will also be set out below in more detail.

In a fourth aspect, the present invention is concerned with a method for modulating or regulating and preferably inhibiting MNK1 and/or MNK2 kinases, wherein said kinases are exposed to at least one compound of formula (I) as defined above in the first aspect (including all preferred embodiments and combinations of embodiments as defined above), wherein said method is preferably performed outside the human or animal body.

In a fifth aspect, the present invention relates to the use of a compound of formula (I) as defined above in the first aspect (including all preferred embodiments and combinations of embodiments as defined above) as MNK1 and/or MNK2 modulating and preferably inhibiting agent.

### DESCRIPTION OF THE FIGURES

FIG. 1: Inhibition of Ser209-eIF4E phosphorylation by compound 2N (example 2N) in prostate cancer cells.

FIG. 2: Inhibition of IL-6 production by compound 2N (example 2N) in murine leukemic cells stimulated with LPS.

### DETAILED DESCRIPTION OF THE INVENTION

The inventors of the present invention inter alia succeeded in identifying new compounds which efficiently inhibit MNK1 and/or MNK2. The compounds of the present invention may thus be particularly used in the treatment of cancer, autoimmune diseases, inflammatory, metabolic and viral diseases.

Before some of the embodiments of the present invention are described in more detail, the following definitions are introduced.

### 1. DEFINITIONS

### General Definitions

As used in the specification and the claims, the singular forms of “a” and “an” also include the corresponding plurals unless the context clearly dictates otherwise. The same applies for plural forms used herein, which also include the singular forms unless the context clearly dictates otherwise.

The terms “about” and “approximately” in the context of the present invention denotes an interval of accuracy that a person skilled in the art will understand to still ensure the technical effect of the feature in question. The term typically indicates a deviation from the indicated numerical value of +10% and preferably +5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group which preferably consists of these embodiments only.

The term “alkyl” refers to a hydrocarbon chain that may be a straight chain or branched chain, containing the indicated number of carbon atoms. For example, C₁₋₆ indicates that the group can have from 1 to 6 (inclusive) carbon atoms in it. If there is no indication of carbon atoms of the alkyl, the term “alkyl” refers to a C₁₋₁₅alkyl, preferably a C₁₋₁₀alkyl, and more preferably to a C₁₋₄alkyl.

In general, the number of carbon atoms present in a given group is designated “Cx-y” where x and y are the lower and upper limits, respectively. For example, a group designated as “C₁₋₅” contains from 1 to 5 (inclusive) carbon atoms. The carbon number as used in the definitions herein refers to carbon backbone and carbon branching, but does not include carbon atoms of the substituents. General examples of alkyl groups include methyl, ethyl, propyl, isopropyl, butyl, n-butyl, isobutyl, sec-butyl, tert-butyl, and pentyl. For example, the term “C₁₋₃alkyl” refers to a straight or branched chain saturated hydrocarbon containing 1-3 carbon atoms. Examples of a C₁₋₃alkyl group include, but are not limited to, methyl, ethyl, propyl and isopropyl.

For example, the term “C₆₋₁₀alkyl” refers to a straight or branched chain saturated hydrocarbon containing 6-10 carbon atoms. Examples of a C₆₋₁₀alkyl group include, but are not limited to, hexyl, octyl and decyl.

“Heteroalkyl” is an alkyl comprising at least one replacement of a CH₂ group independently by O, S or NT1, wherein in a C₃₋₄alkyl one of the CH₂ groups of the C₃₋₄alkyl, excluding the first and the last carbon atom, is replaced by O, S or NT1, and wherein in a C₅₋₆alkyl either one or two non-adjacent CH₂ groups of the C₅₋₆alkyl, excluding the first and the last carbon atom, is/are replaced independently by O, S or NT1. T1 is defined as given in the first aspect of the present invention.

“Alkenyl” is a hydrocarbon chain having at least one (preferably only one) carbon-carbon double bond. “Alkynyl” is a hydrocarbon chain having at least one (preferably only one) carbon-carbon triple bond.

The term “carbocyclic ring system” refers to a cyclic structure comprising only carbon atoms as ring atoms. This system can be mono- or bicyclic; i) saturated or partially unsaturated non-aromatic or ii) aromatic; and comprise a number of total ring carbon atoms as indicated. Examples (given as radicals) of i) include cyclopropane, cyclopentane, cyclohexane and cyclohexene and of ii) naphthalenyl and phenyl, wherein phenyl is preferred.

The term “heterocyclic ring system” refers to a cyclic structure comprising carbon atoms as ring atoms and at least one heteroatom as ring atom, with the upper number of heteroatoms as ring atoms as indicated. The term “hetero atom” as used herein preferably refers to nitrogen, sulfur and oxygen atoms. A heterocyclic ring system may generally contain different heteroatoms. For the present invention, nitrogen as heteroatom may be preferred. Further, for the present invention, it can be preferred that a heterocycle comprises one or two heteroatoms. This system can be mono- or bicyclic; i) saturated or partially unsaturated non-aromatic or ii) aromatic; and comprise a number of total ring atoms as indicated. Examples (given as radicals) of i) include oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl and thiazinyl. Examples (given as radicals) of ii) include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, imidazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thia-diazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl.

The term “halogen” includes fluorine, bromine, chlorine or iodine. The term “amino” represents —NH₂, the term “hydroxyl” is —OH, the term “thiol” is —SH, the term “nitro” is —NO₂—, the term “cyano” is —CN and “oxo” is ═O. “Carbon branching” or “branched alkyl” means that one or more alkyl groups such as methyl, ethyl or propyl, replace one or both hydrogens in a —CH₂— group of a linear alkyl chain.

It is usually indicated herein whether a given substituent is unsubstituted or substituted with a specific further substituent. If the present application should not directly and unambiguously indicate a substitution, this usually means that the respective substituent is unsubstituted. Thus, if e.g. reference is made to “C₁₋₆alkyl” or “C₂₋₆alkenyl” without any further indication, this usually means “unsubstituted C₁₋₆alkyl” or “unsubstituted C₂₋₆alkenyl”.

The invention disclosed herein is meant to encompass all salts and particularly pharmaceutically acceptable salts of compound (I), particularly the salts referred to above. Further, the pharmaceutically acceptable salts include metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, fumarate, tartrate and the like; sulfonates such as methanesulfonate, benzenesulfonate, p-toluenesulfonate, and the like; amino acid salts such as arginate, asparginate, glutamate and the like. A particularly preferred pharmaceutically acceptable salt is selected from the group consisting of the hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate. The hydrochloride salt is particularly preferred for compounds of the present invention.

The compounds disclosed herein may contain one or more asymmetric centers and may thus lead to enantiomers, diastereomers, and other stereoisomeric forms. The present invention is also meant to encompass all such possible forms as well as their racemic and resolved forms and mixtures thereof, unless specified otherwise. When the compounds described herein contain olefinic double bonds or other centers of geometric asymmetry, and unless specified otherwise, it is intended to include both E and Z geometric isomers. All tautomers are intended to be encompassed by the present invention as well. As used herein, the term “stereoisomers” is a general term for all isomers of individual molecules that differ only in the orientation of their atoms in space. It includes enantiomers and isomers of compounds with more than one chiral center that are not mirror images of one another (diastereomers). The term “chiral center” refers to an atom to which four different groups are attached. The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction. The term “racemic” refers to a mixture of equal parts of enantiomers and which is optically inactive. The term “resolution” refers to the separation or concentration or depletion of one of the two enantiomeric forms of a molecule.

The term “N-oxide” includes any compound of formula (I) which has at least one tertiary nitrogen atom that is oxidized to an N-oxide moiety.

“Pharmaceutically active agent” as used herein means that a compound is potent of modulating a response in a human or animal being in vivo. When reference is made to a compound as “the only pharmaceutically active agent”, this is meant to describe that the activity of a corresponding pharmaceutical composition is due to said active agent only.

The term “pharmaceutically acceptable excipient” as used herein refers to compounds commonly comprised in pharmaceutical compositions, which are known to the skilled person. Such compounds or excipients are exemplary listed below. In view of the definition “pharmaceutically active agent” as given above, a pharmaceutically acceptable excipient can be defined as being pharmaceutically inactive.

The term “treatment” is to be understood as also including the option of “prophylaxis”. Thus, whenever reference is made herein to a “treatment” or “treating”, this is to be understood as “treatment and/or prophylaxis” or “treating and/or preventing”.

### Description of Pharmaceutical Compositions According to the Present Invention

A pharmaceutical composition according to the present invention may be formulated for oral, buccal, nasal, rectal, topical, transdermal or parenteral application. Oral application may be preferred. Parenteral application can also be preferred and includes intravenous, intramuscular or subcutaneous administration. The compound according to formula (I) should be applied in pharmaceutically effective amounts, for example in the amounts as set out herein below.

A pharmaceutical composition of the present invention may also be designated as formulation or dosage form. A compound of formula (I) may also be designated in the following as (pharmaceutically) active agent or active compound.

Pharmaceutical compositions may be solid or liquid dosage forms or may have an intermediate, e.g. gel-like character depending inter alia on the route of administration.

In general, the inventive dosage forms can comprise various pharmaceutically acceptable excipients which will be selected depending on which functionality is to be achieved for the dosage form. A “pharmaceutically acceptable excipient” in the meaning of the present invention can be any substance used for the preparation of pharmaceutical dosage forms, including coating materials, film-forming materials, fillers, disintegrating agents, release-modifying materials, carrier materials, diluents, binding agents and other adjuvants. Typical pharmaceutically acceptable excipients include substances like sucrose, mannitol, sorbitol, starch and starch derivatives, lactose, and lubricating agents such as magnesium stearate, disintegrants and buffering agents.

The term “carrier” denotes pharmaceutically acceptable organic or inorganic carrier substances with which the active ingredient is combined to facilitate the application. Suitable pharmaceutically acceptable carriers include, for instance, water, salt solutions, alcohols, oils, preferably vegetable oils, polyethylene glycols, gelatin, lactose, amylose, magnesium stearate, surfactants, perfume oil, fatty acid monoglycerides and diglycerides, petroethral fatty acid esters, hydroxymethyl-cellulose, polyvinylpyrrolidone and the like. The pharmaceutical compositions can be sterilized and if desired, mixed with auxiliary agents, like lubricants, preservatives, stabilizers, wetting agents, emulsifiers, salts for influencing osmotic pressure, buffers, colorings, flavoring and/or aromatic substances and the like which do not deleteriously react with the active compound.

If liquid dosage forms are considered for the present invention, these can include pharmaceutically acceptable emulsions, solutions, suspensions and syrups containing inert diluents commonly used in the art such as water. These dosage forms may contain e.g. microcrystalline cellulose for imparting bulk, alginic acid or sodium alginate as a suspending agent, methylcellulose as a viscosity enhancer and sweeteners/flavouring agents.

For parenteral application, particularly suitable vehicles consist of solutions, preferably oily or aqueous solutions, as well as suspensions, emulsions, or implants. Pharmaceutical formulations for parenteral administration are particularly preferred and include aqueous solutions of the compounds of formula (I) in water-soluble form. Additionally, suspensions of the compounds of formula (I) may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

Particularly preferred dosage forms are injectable preparations of a compound of formula (I). Thus, sterile injectable aqueous or oleaginous suspensions can for example be formulated according to the known art using suitable dispersing agents, wetting agents and/or suspending agents. A sterile injectable preparation can also be a sterile injectable solution or suspension in a non-toxic parenterally acceptable diluant or solvent. Among the acceptable vehicles and solvents that can be used are water and isotonic sodium chloride solution. Sterile oils are also conventionally used as solvent or suspending medium.

Suppositories for rectal administration of a compound of formula (I) can be prepared by e.g. mixing the compound with a suitable non-irritating excipient such as cocoa butter, synthetic triglycerides and polyethylene glycols which are solid at room temperature but liquid at rectal temperature such that they will melt in the rectum and release the compound according to formula (I) from said suppositories.

For administration by inhalation, the compounds according to the present invention may be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Oral dosage forms may be liquid or solid and include e.g. tablets, troches, pills, capsules, powders, effervescent formulations, dragees and granules. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. The oral dosage forms may be formulated to ensure an immediate release of the compound of formula (I) or a sustained release of the compound of formula (I).

A solid dosage form may comprise a film coating. For example, the inventive dosage form may be in the form of a so-called film tablet. A capsule of the invention may be a two-piece hard gelatin capsule, a two-piece hydroxypropylmethylcellulose capsule, a two-piece capsule made of vegetable or plant-based cellulose or a two-piece capsule made of polysaccharide.

The dosage form according to the invention may be formulated for topical application. Suitable pharmaceutical application forms for such an application may be a topical nasal spray, sublingual administration forms and controlled and/or sustained release skin patches. For buccal administration, the compositions may take the form of tablets or lozenges formulated in conventional manner.

The compositions may conveniently be presented in unit dosage forms and may be prepared by any of the methods well known in the art of pharmacy. The methods can include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

As regards human patients, the compound of formula (I) may be administered to a patient in an amount of about 0.001 mg to about 5000 mg per day, preferably of about 0.01 mg to about 100 mg per day, more preferably of about 0.1 mg to about 50 mg per day, which is the effective amount. The phrase “effective amount” means an amount of compound that, when administered to a mammal in need of such treatment, is sufficient to treat or prevent a particular disease or condition.

Indications, for which the Compounds of the Present Invention May be Used

The compounds according to the present invention are preferably used for the treatment of a disease selected from the group consisting of oncogenic (in particular hematopoietic diseases), metabolic, inflammatory, autoimmune and viral diseases.

Thus, in one embodiment, the compounds of the present invention are useful for the treatment of cancer, such as cancer of the upper gastrointestinal tract, pancreatic carcinoma, breast cancer, colon cancer, ovarian carcinoma, cervix carcinoma, endometrial cancer, brain tumor, testicular cancer, laryngeal carcinoma, osteocarcinoma, prostatic cancer, retinoblastoma, liver carcinoma, lung cancer, neuroblastoma, renal carcinoma, thyroid carcinoma, esophageal cancer, soft tissue sarcoma, skin cancer, osteosarcoma, rhabdomyosarcoma, bladder cancer or metastatic cancer.

In a preferred embodiment, the compounds of the present invention are useful for the treatment of hematopoietic disorders, such as acute myeloid leukemia (AML), Morbus Hodgkin, Non-Hodgkin's lymphoma, hematopoietic disease, acute non-lymphocytic leukemia (ANLL), myeloproliferative disease acute promyelocytic leukemia (APL), acute myelomonocytic leukemia (AMMoL), multiple myeloma, polycythemia vera, lymphoma, acute lymphocytic leukemia (ALL), chronic lymphocytic leukemia (CCL), Wilm's tumor, or Ewing's Sarcoma.

Metabolic diseases are diseases caused by an abnormal metabolic process and may either be congenital due to an inherited enzyme abnormality or acquired due to a disease of an endocrine organ or failure of a metabolically important organ such as the liver or the pancreas. The present invention is directed to the compound of formula (I) for use in the treatment of in particular metabolic diseases of the lipid and carbohydrate metabolism. Lipid disorders cover a group of conditions which cause abnormalities in the level and metabolism of plasma lipids and lipoproteins.

Diabetes mellitus is defined as a chronic hyperglycemia associated with resulting damages to organs and dysfunctions of metabolic processes. Depending on its etiology, one differentiates between several forms of diabetes, which are either due to an absolute (lacking or decreased insulin secretion) or to a relative lack of insulin. Diabetes mellitus Type I (IDDM, insulin-dependent diabetes mellitus) generally occurs in adolescents under 20 years of age. It is assumed to be of auto-immune etiology, leading to an insulitis with the subsequent destruction of the beta cells of the islets of Langerhans which are responsible for the insulin synthesis. In addition, in latent autoimmune diabetes in adults, beta cells are being destroyed due to autoimmune attack. The amount of insulin produced by the remaining pancreatic islet cells is too low, resulting in elevated blood glucose levels (hyperglycemia). Diabetes mellitus Type II generally occurs at an older age. It is above all associated with a resistance to insulin in the liver and the skeletal muscles, but also with a defect of the islets of Langerhans. High blood glucose levels (and also high blood lipid levels) in turn lead to an impairment of beta cell function and to an increase in beta cell apoptosis.

Diabetes is a very disabling disease, because today's common anti-diabetic drugs do not control blood sugar levels well enough to completely prevent the occurrence of high and low blood sugar levels. Out of range blood sugar levels are toxic and cause long-term complications for example retinopathy, tenopathy, neuropathy and peripheral vascular disease. There is also a host of related conditions, such as obesity, hypertension, heart disease and hyperlipidemia, for which persons with diabetes are substantially at risk. Obesity is associated with an increased risk of follow-up diseases such as cardiovascular diseases, hypertension, diabetes, hyperlipidemia and an increased mortality. Diabetes (insulin resistance) and obesity are part of the “metabolic syndrome” which is defined as the linkage between several diseases. These often occur in the same patients and are major risk factors for development of diabetes type II and cardiovascular disease.

In another embodiment, the compounds of the present invention are useful for the treatment of metabolic diseases of the carbohydrate metabolism and their consecutive complications and disorders such as impaired glucose tolerance, diabetes (preferably diabetes type II), diabetic complications such as diabetic gangrene, diabetic arthropathy, diabetic osteopenia, diabetic glomerosclerosis, diabetic nephropathy, diabetic dermopathy, diabetic neuropathy, diabetic cataract and diabetic retinopathy, diabetic maculopathy, diabetic feet syndrome, diabetic coma with or without ketoacidosis, diabetic hyperosmolar coma, hypoglycemic coma, hyperglycemic coma, diabetic acidosis, diabetic ketoacidosis, intracapillary glomerulonephrosis, Kimmelstiel-Wilson syndrome, diabetic amyotrophy, diabetic autonomic neuropathy, diabetic mononeuropathy, diabetic polyneuropathy, diabetic angiopathies, diabetic peripheral angiopathy, diabetic ulcer, diabetic arthropathy, or obesity in diabetes.

In a further embodiment, the compounds of the present invention are useful for the treatment of metabolic diseases of the lipid metabolism, such as hypercholesterolemia, familial hypercholesterolemia, Fredrickson's hyperlipoproteinemia, hyperbetalipoproteinemia, hyperlipidemia, low-density-lipoprotein-type [LDL] hyperlipoproteinemia, pure hyperglyceridemia, endogenous hyperglyceridemia, isolated hypercholesterolemia, isolated hypertroglyceridemia, cardiovascular diseases such as hypertension, ischemia, varicose veins, retinal vein occlusion, atherosclerosis, angina pectoris, myocardial infarction, stenocardia, pulmonary hypertension, congestive heart failure, glomerulopaty, tubulointestitial disorders, renal failure, angiostenosis, or cerebrovascular disorders, such as cerebral apoplexy.

The present invention also relates to treatment of cytokine-related diseases with compounds of the present invention. Such diseases are e.g. inflammatory diseases, autoimmune diseases, destructive bone disorders, proliferative disorders, infectious diseases, neurodegenerative diseases, or allergies, in particular allergic and inflammatory diseases, such as acute or chronic inflammation, chronic inflammatory arthritis, rheumatoid arthritis, psoriasis, COPD, inflammatory bowel disease, asthma and septic shock. The compounds of the present invention are useful for the treatment of inflammatory diseases, such as chronic or acute inflammation, inflammation of the joints such as chronic inflammatory arthritis, rheumatoid arthritis, psoriatic arthritis, osteoarthritis, juvenile rheumatoid arthritis, Reiter's syndrome, rheumatoid traumatic arthritis, rubella arthritis, acute synovitis and gouty arthritis; inflammatory skin diseases such as sunburn, psoriasis, erythrodermic psoriasis, pustular psoriasis, eczema, dermatitis, acute or chronic graft formation, atopic dermatitis, contact dermatitis, urticaria and scleroderma; inflammation of the gastrointestinal tract such as inflammatory bowel disease, Crohn's disease and related conditions, ulcerative colitis, colitis, and diverticulitis; nephritis, urethritis, salpingitis, oophoritis, endomyometritis, spondylitis, systemic lupus erythematosus and related disorders, multiple sclerosis, asthma, meningitis, myelitis, encephalomyelitis, encephalitis, phlebitis, thrombophlebitis, respiratory diseases such as asthma, bronchitis, chronic obstructive pulmonary disease (COPD), inflammatory lung disease and adult respiratory distress syndrome, and allergic rhinitis; endocarditis, osteomyelitis, rheumatic fever, rheumatic pericarditis, rheumatic endocarditis, rheumatic myocarditis, rheumatic mitral valve disease, rheumatic aortic valve disease, prostatitis, prostatocystitis, spondoarthropathies ankylosing spondylitis, synovitis, tenosynovotis, myositis, pharyngitis, polymyalgia rheumatica, shoulder tendonitis or bursitis, gout, pseudo gout, vasculitides, inflammatory diseases of the thyroid selected from granulomatous thyroiditis, lymphocytic thyroiditis, invasive fibrous thyroiditis, acute thyroiditis; Hashimoto's thyroiditis, Kawasaki's disease, Raynaud's phenomenon, Sjogren's syndrome, neuroinflammatory disease, sepsis, conjunctivitis, keratitis, iridocyclitis, optic neuritis, otitis, lymphoadenitis, nasopaharingitis, sinusitis, pharyngitis, tonsillitis, laryngitis, epiglottitis, bronchitis, pneumonitis, stomatitis, gingivitis, oesophagitis, gastritis, peritonitis, hepatitis, cholelithiasis, cholecystitis, glomerulonephritis, goodpasture's disease, crescentic glomerulonephritis, pancreatitis, endomyometritis, myometritis, metritis, cervicitis, endocervicitis, exocervicitis, parametritis, tuberculosis, vaginitis, vulvitis, silicosis, sarcoidosis, pneumoconiosis, pyresis, inflammatory polyarthropathies, psoriatric arthropathies, intestinal fibrosis, bronchiectasis and enteropathic arthropathies.

Moreover, cytokines are also believed to be implicated in the production and development of various cardiovascular and cerebrovascular disorders such as congestive heart disease, myocardial infarction, the formation of atherosclerotic plaques, hypertension, platelet aggregation, angina, stroke, Alzheimer's disease, reperfusion injury, vascular injury including restenosis and peripheral vascular disease, and, for example, various disorders of bone metabolism such as osteoporosis (including senile and postmenopausal osteoporosis), Paget's disease, bone metastases, hypercalcaemia, hyperparathyroidism, osteosclerosis, osteoporosis and periodontitis, and the abnormal changes in bone metabolism which may accompany rheumatoid arthritis and osteoarthritis. The treatment of these diseases by compounds of the present invention is also within the scope of the present invention.

Excessive cytokine production has also been implicated in mediating certain complications of bacterial, fungal and/or viral infections such as endotoxic shock, septic shock and toxic shock syndrome and in mediating certain complications of CNS surgery or injury such as neurotrauma and ischaemic stroke. Excessive cytokine production has, moreover, been implicated in mediating or exacerbating the development of diseases involving cartilage or muscle resorption, pulmonary fibrosis, cirrhosis, renal fibrosis, the cachexia found in certain chronic diseases such as malignant disease and acquired immune deficiency syndrome (AIDS), tumour invasiveness and tumour metastasis and multiple sclerosis. The treatment of these diseases is also contemplated by the present invention.

Additionally, the inventive compounds may be used to treat inflammation associated with autoimmune diseases including systemic lupus erythematosis, Addison's disease, autoimmune polyglandular disease (also known as autoimmune polyglandular syndrome), glomerulonephritis, rheumatoid arthritis scleroderma, chronic thyroiditis, Graves' disease, autoimmune gastritis, diabetes, autoimmune hemolytic anemia, glomerulonephritis, rheumatoid arthritis autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, and graft vs. host disease.

In a further embodiment, the compounds of the present invention may be used for the treatment of infectious diseases such as sepsis, septic shock, Shigellosis, and Helicobacter pylori and viral diseases including herpes simplex type 1 (HSV-1), herpes simplex type 2 (HSV-2), cytomegalovirus, Epstein-Barr, human immunodeficiency virus (HIV), acute hepatitis infection (including hepatitis A, hepatitis B, and hepatitis C), HIV infection and CMV retinitis, AIDS or malignancy, malaria, mycobacterial infection and meningitis. These also include viral infections, by influenza virus, varicella-zoster virus (VZV), Epstein-Barr virus, human herpesvirus-6 (HHV-6), human herpesvirus-7 (HHV-7), human herpesvirus-8 (HHV-8), Poxvirus, Vacciniavirus, Monkeypoxvirus, pseudorabies and rhinotracheitis.

The compounds of the present invention may also be used (preferably topically) in the treatment of topical diseases mediated by or exacerbated by excessive cytokine production, such as inflamed joints, eczema, psoriasis and other inflammatory skin conditions such as sunburn; inflammatory eye conditions including conjunctivitis; pyresis, and pain. Periodontal disease has also been implemented in cytokine production, both topically and systemically. Hence, treatment of an inflammation associated with cytokine production in such peroral diseases such as gingivitis and periodontitis is within the scope of the present invention.

The compounds of the present invention may also be used to treat a neurodegenerative disease selected from Alzheimer's disease, Parkinson's disease, amyotrophic lateral sclerosis, Huntington's disease, frontotemporal lobar dementia, spinocerebellar ataxia, dementia with Lewy bodies, cerebral ischemia or neurodegenerative disease caused by traumatic injury, glutamate neurotoxicity or hypoxia.

Finally, certain anti-cancer drugs such as cisplatin are linked to serious side effects such as nephrotoxicity or ototoxicity, which can be dose limiting. Activation of MNKs has been linked to these side effects. In a further embodiment of the present invention, the compounds of the present invention are useful for the treatment of ear or kidney damage, in particular for the treatment of ear and kidney drug induced damage.

In a preferred embodiment relating to the pharmaceutical compositions of the present invention, said pharmaceutical composition comprises said compound as the only pharmaceutically active agent. Alternatively, said pharmaceutical composition comprises at least one further independent pharmaceutically active agent in addition to said compound, wherein said additional active agent is typically used for the intended indication(s) as outlined above.

Further, the compounds of the present invention may be useful as adjuvants to e.g. cancer treatment. They may be used in combination with one or more additional drugs, for example a chemotherapeutic agent which acts by the same or by a different mechanism of action. Such drugs are listed in the example section of the present application and comprise both targeted agents such as kinase inhibitors of the PI3K/Akt/mTOR pathway or the JAK/STAT pathway, but also standard chemotherapy agents such as cytarabine, and vosaroxin. In particular, the compounds of preferred embodiments (A) stated above may be used in cancer therapy (e.g. for use in treating acute myelogenous leukemia (AML), diffuse large B-cell lymphoma (DLBCL) and multiple myeloma (MM)) in combination with a chemotherapeutic agent such as a PI3K inhibitor, a JAK kinase inhibitor, cytarabine, vosaroxin and combinations thereof. Other targeted cancer therapy agents such as e.g. kinase inhibitors may, however, also be used in combination with compounds of the present invention.

### 2. ALTERNATIVE FORMULATIONS

The subject matter of the present invention may also be referred to as follows: Method of administering to a subject in need thereof an effective amount of a compound according to formula (I) as defined above (including all embodiments and combinations thereof).

Method of treating a disease selected from the disease as disclosed herein by administering to a subject in need thereof an effective amount of a compound according to formula (I) as defined above (including all embodiments and combinations thereof).

Method for treating a MNK1 and/or MNK2-related disease and/or disorder, said method comprising the step of administering to a patient in need thereof a therapeutic amount of a compound according to formula (I) thereof as defined above (including all embodiments and combinations thereof).

Method for treating a MNK1 and/or MNK2-related cancer, said method comprising the step of administering to a patient in need thereof a therapeutic amount of a compound according to formula (I) as defined above (including all embodiments and combinations thereof).

Method for treating a MNK1 and/or MNK2-related metabolic disease and/or disorder, said method comprising the step of administering to a patient in need thereof a therapeutic amount of a compound according to formula (I) as defined above (including all embodiments and combinations thereof).

Method for treating a MNK1 and/or MNK2-related inflammatory disease and/or disorder, said method comprising the step of administering to a patient in need thereof a therapeutic amount of a compound according to formula (I) as defined above (including all embodiments and combinations thereof).

Method for treating a MNK1 and/or MNK2-related autoimmune disease and/or disorder, said method comprising the step of administering to a patient in need thereof a therapeutic amount of a compound according to formula (I) as defined above (including all embodiments and combinations thereof).

Method for treating a MNK1 and/or MNK2-related viral disease and/or disorder, said method comprising the step of administering to a patient in need thereof a therapeutic amount of a compound according to formula (I) as defined above (including all embodiments and combinations thereof).

In the following, examples of embodiments of the present invention are outlined. However, said examples should not be construed as limiting the scope of the present invention.

### 3. EXAMPLES

3.1. Compounds of the present application:

### Example 1A

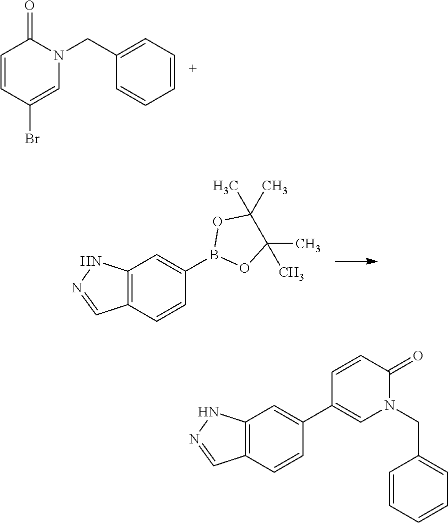 [SMILES: O=c1ccc(Br)cn1Cc1ccccc1 ; CC1(C)OB(c2ccc3cn[nH]c3c2)OC1(C)C ; O=c1ccc(-c2ccc3cn[nH]c3c2)cn1Cc1ccccc1]

### 1-benzyl-5-(1H-indazol-6-yl)-1,2-dihydropyridin-2-one

A 1-benzyl-5-bromo-1,2-dihydropyridin-2-one (Method 1A) (0.1 g, 0.4 mmol), 6-(tetramethyl-1,3,2-dioxaborolan-2-yl)-1H-indazole (Method 3A) (0.18 g, 0.8 mmol), caesium carbonate (0.4 g, 1.1 mmol) in mixture dioxane/water (2:1) (1.5 mL) were flushed with argon for 10 min and [1,1′-bis(diphenylphosphino)ferrocene]palladium(II) chloride, complex with dichloromethane (0.03 g) was added. The reaction mixture was heated at 125° C. under microwave irradiation until the reaction was completed. After cooled to ambient temperature reaction mixture was filtered through Celite, washed with ethyl acetate and solvents were evaporated under reduced pressure. Crude product was purified by flash chromatography (dichloromethane/methanol 95:5) to give 1-benzyl-5-(1H-indazol-6-yl)-1,2-dihydropyridin-2-one (0.06 g); yield 63%. LC-MS (m/z) 302.1 (M+1). ¹H NMR (300 MHz, DMSO) δ 13.09 (s, 1H), 8.31 (d, J=2.5 Hz, 1H), 8.04 (s, 1H), 7.90 (dd, J=9.5, 2.7 Hz, 1H), 7.78 (d, J=8.4 Hz, 1H), 7.62 (s, 1H), 7.39-7.24 (m, 6H), 6.53 (d, J=9.4 Hz, 1H), 5.19 (s, 2H).

The following examples were prepared by the procedure of Example 1A, using the appropriate starting materials.

| Ex. | Product | ¹H NMR | m/z | Starting materials |
|---|---|---|---|---|
| 1B | 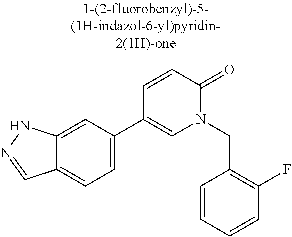 [SMILES: O=c1ccc(-c2ccc3cn[nH]c3c2)cn1Cc1ccccc1F] | (400 MHz, DMSO) δ 13.13 (s, 1H), 8.28 (d, J = 2.6 Hz, 1H), 8.08 (s, 1H), 7.96 (dd, J = 9.5, 2.7 Hz, 1H), 7.82 (d, J = 8.4 Hz, 1H), 7.66 (s, 1H), 7.39-7.31 (m, 2H), 7.22 (ddd, J = 10.5, 9.5, 6.3 Hz, 3H), 6.56 (d, J = 9.5 Hz, 1H), 5.27 (s,2H) | 320.0 | 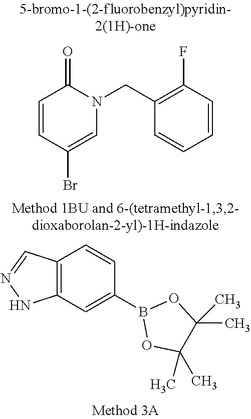 [SMILES: O=c1ccc(Br)cn1Cc1ccccc1F ; CC1(C)OB(c2ccc3cn[nH]c3c2)OC1(C)C] |
| 1C | 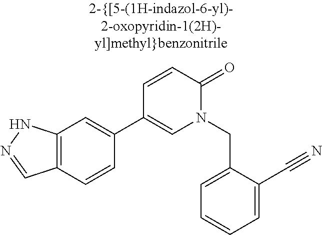 [SMILES: N#Cc1ccccc1Cn1cc(-c2ccc3cn[nH]c3c2)ccc1=O] | | 327.3 | 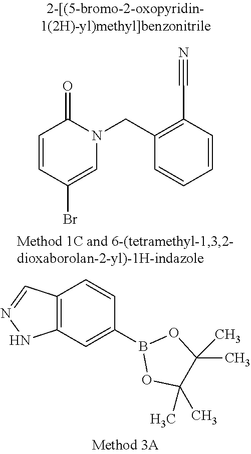 [SMILES: N#Cc1ccccc1Cn1cc(Br)ccc1=O ; CC1(C)OB(c2ccc3cn[nH]c3c2)OC1(C)C] |
| 1D | 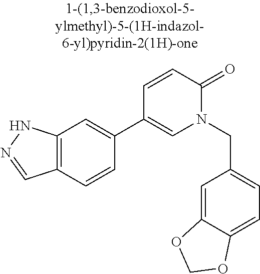 [SMILES: O=c1ccc(-c2ccc3cn[nH]c3c2)cn1Cc1ccc2c(c1)OCO2] | (400 MHz, DMSO) δ 13.13 (s, 1H), 8.32 (d, J = 2.5 Hz, 1H), 8.07 (s, 1H), 7.90 (dd, J = 9.5, 2.7 Hz, 1H), 7.81 (d, J = 8.4 Hz, 1H), 7.64 (s, 1H), 7.33 (d, J = 8.5 Hz, 1H), 7.06 (d, J = 1.3 Hz, 1H), 6.97-6.94 (m, 1H), 6.89 (d, J = 8.0 Hz, 1H), 6.54 (d, J = 9.4 Hz, 1H), 5.99 (s, 2H), 5.10 (s, 2H) | 346.1 | 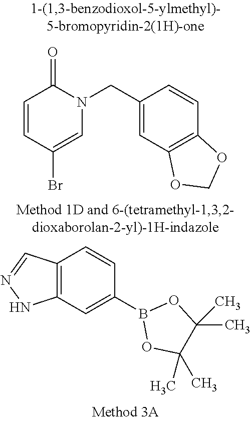 [SMILES: O=c1ccc(Br)cn1Cc1ccc2c(c1)OCO2 ; CC1(C)OB(c2ccc3cn[nH]c3c2)OC1(C)C] |
| 1F | 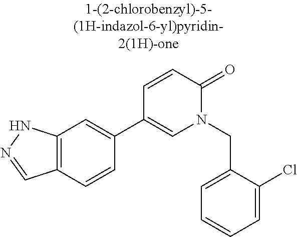 [SMILES: O=c1ccc(-c2ccc3cn[nH]c3c2)cn1Cc1ccccc1Cl] | (400 MHz, DMSO-d6) δ 13.12 (s, 1H), 8.27 (d, J = 2.6 Hz, 1H), 8.10-8.04 (m, 1H), 8.01 (dd, J = 9.5, 2.7 Hz, 1H), 7.81 (d, J = 8.4 Hz, 1H), 7.66 (s, 1H), 7.56-7.49 (m, 1H), 7.39-7.28 (m, 3H), 7.00-6.91 (m, 1H), 6.61 (d, J = 9.5 Hz, 1H). | 336.3 | 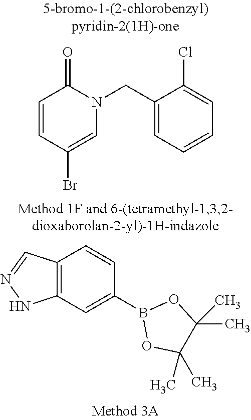 [SMILES: O=c1ccc(Br)cn1Cc1ccccc1Cl ; CC1(C)OB(c2ccc3cn[nH]c3c2)OC1(C)C] |
| 1G | 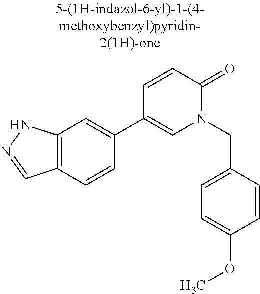 [SMILES: COc1ccc(Cn2cc(-c3ccc4cn[nH]c4c3)ccc2=O)cc1] | (400 MHz, DMSO) δ 13.12 (s, 1H), 8.18 (d, J = 2.6 Hz, 1H), 8.07 (s, 1H), 7.94 (dd, J = 9.5, 2.7 Hz, 1H), 7.80 (d, J = 8.4 Hz, 1H), 7.64 (s, 1H), 7.35- 7.25 (m, 2H), 7.05 (d, J = 8.0 Hz, 1H), 6.99-6.88 (m, 2H), 6.55 (d, J = 9.4 Hz, 1H), 5.16 (s, 2H), 3.87 (s, 3H) | 333.1 | 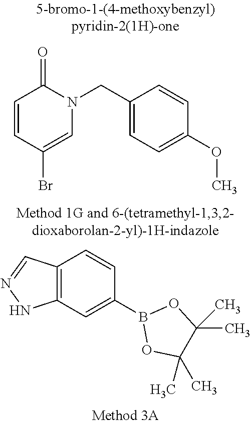 [SMILES: COc1ccc(Cn2cc(Br)ccc2=O)cc1 ; CC1(C)OB(c2ccc3cn[nH]c3c2)OC1(C)C] |
| 1H | 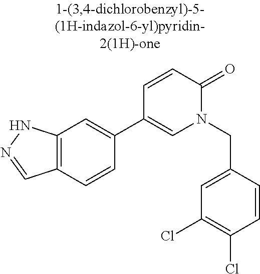 [SMILES: O=c1ccc(-c2ccc3cn[nH]c3c2)cn1Cc1ccc(Cl)c(Cl)c1] | (400 MHz, DMSO) δ 13.15 (s, 1H), 8.40 (d, J = 2.6 Hz, 1H), 8.08 (s, 1H), 7.95 (dd, J = 9.5, 2.6 Hz, 1H), 7.82 (d, J = 8.4 Hz, 1H), 7.70 (d, J = 1.9 Hz, 1H), 7.67 (s, 1H), 7.64-7.61 (m, 1H), 7.40 (dd, J = 8.3, 2.0 Hz, 1H), 7.35 (dd, J = 8.4, 1.3 Hz, 1H), 6.57 (d, J = 9.5 Hz, 1H), 5.19 (s, 2H) | 370.4 | 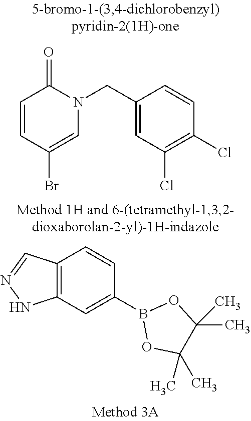 [SMILES: O=c1ccc(Br)cn1Cc1ccc(Cl)c(Cl)c1 ; CC1(C)OB(c2ccc3cn[nH]c3c2)OC1(C)C] |
| 1I | 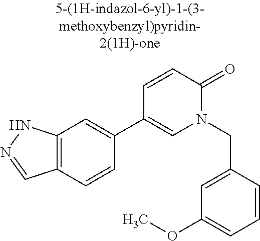 [SMILES: COc1cccc(Cn2cc(-c3ccc4cn[nH]c4c3)ccc2=O)c1] | (400 MHz, DMSO) δ 13.12 (s, 1H), 8.31 (d, J = 2.7 Hz, 1H), 8.07 (s, 1H), 7.92 (dd, J = 9.4, 2.7 Hz, 1H), 7.80 (d, J = 8.4 Hz, 1H), 7.64 (s, 1H), 7.33 (dd, J = 8.5, 1.4 Hz, 1H), 7.27 (t, J = 7.9 Hz, 1H), 6.97 (d, J = 1.8 Hz, 1H), 6.94 (d, J = 7.7 Hz, 1H), 6.86 (dd, J= 8.2, 2.5 Hz, 1H), 6.56 (d, J = 9.5 Hz, 1H), 5.18 (s, 2H), 3.73 (s, 3H) | 332.1 | 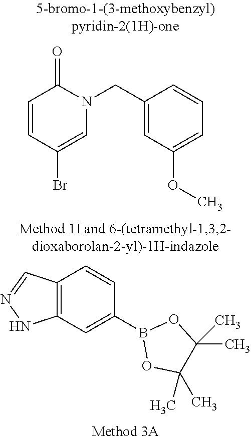 [SMILES: COc1cccc(Cn2cc(Br)ccc2=O)c1 ; CC1(C)OB(c2ccc3cn[nH]c3c2)OC1(C)C] |
| 1J | 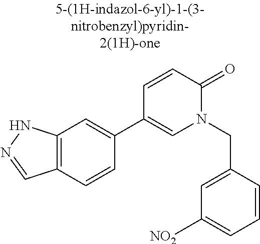 [SMILES: O=c1ccc(-c2ccc3cn[nH]c3c2)cn1Cc1cccc([N+](=O)[O-])c1] | | 347.1 | 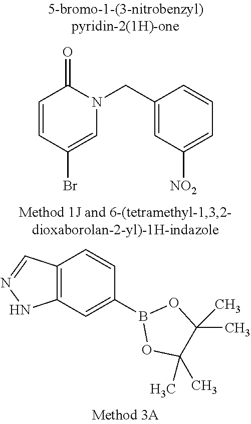 [SMILES: O=c1ccc(Br)cn1Cc1cccc([N+](=O)[O-])c1 ; CC1(C)OB(c2ccc3cn[nH]c3c2)OC1(C)C] |
| 1K | 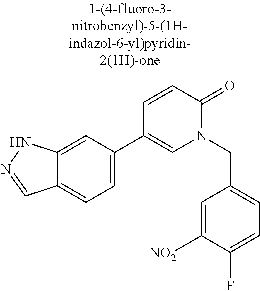 [SMILES: O=c1ccc(-c2ccc3cn[nH]c3c2)cn1Cc1ccc(F)c([N+](=O)[O-])c1] | | 365.1 | 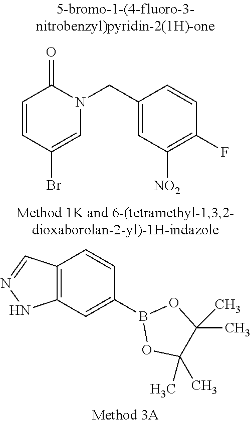 [SMILES: O=c1ccc(Br)cn1Cc1ccc(F)c([N+](=O)[O-])c1 ; CC1(C)OB(c2ccc3cn[nH]c3c2)OC1(C)C] |
| 1M | 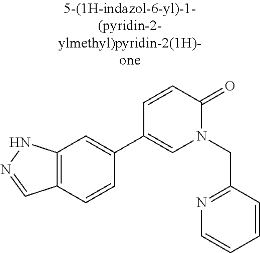 [SMILES: O=c1ccc(-c2ccc3cn[nH]c3c2)cn1Cc1ccccn1] | | 303.1 | 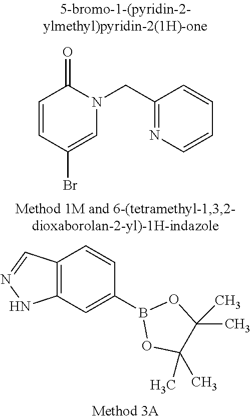 [SMILES: O=c1ccc(Br)cn1Cc1ccccn1 ; CC1(C)OB(c2ccc3cn[nH]c3c2)OC1(C)C] |
| 1N | 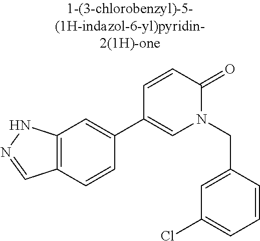 [SMILES: O=c1ccc(-c2ccc3cn[nH]c3c2)cn1Cc1cccc(Cl)c1] | (400 MHz, DMSO-d6) δ 13.12 (s, 1H), 8.38 (d, J = 2.6 Hz, 1H), 8.07 (s, 1H), 7.94 (dd, J = 9.5, 2.7 Hz, 1H), 7.81 (d, J = 8.4 Hz, 1H), 7.66 (s, 1H), 7.47 (s, 1H), 7.41-7.32 (m, 4H), 6.57 (d, J = 9.5 Hz, 1H), 5.21 (s, 2H). | 336.2 | 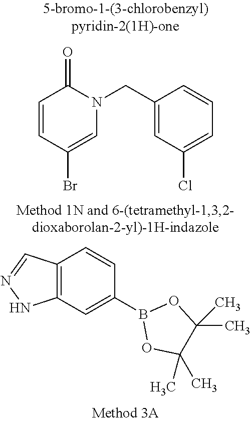 [SMILES: O=c1ccc(Br)cn1Cc1cccc(Cl)c1 ; CC1(C)OB(c2ccc3cn[nH]c3c2)OC1(C)C] |
| 1O | 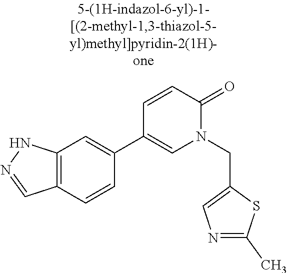 [SMILES: Cc1ncc(Cn2cc(-c3ccc4cn[nH]c4c3)ccc2=O)s1] | | 323.0 | 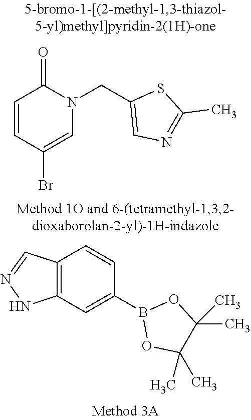 [SMILES: Cc1ncc(Cn2cc(Br)ccc2=O)s1 ; CC1(C)OB(c2ccc3cn[nH]c3c2)OC1(C)C] |
| 1S | 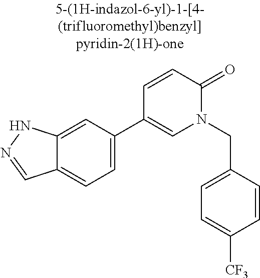 [SMILES: O=c1ccc(-c2ccc3cn[nH]c3c2)cn1Cc1ccc(C(F)(F)F)cc1] | (400 MHz, DMSO) δ 13.14 (s, 1H), 8.40 (d, J = 2.6 Hz, 1H), 8.08 (s, 1H), 7.96 (dd, J = 9.5, 2.7 Hz, 1H), 7.81 (d, J = 8.5 Hz, 1H), 7.73 (d, J = 8.2 Hz, 2H), 7.67 (s, 1H), 7.58 (d, J = 8.1 Hz, 2H), 7.35 (dd, J = 8.5, 1.3 Hz, 1H), 6.56 (t, J = 10.8 Hz, 1H), 5.31 (s, 2H), 1.49-1.29 (m, 2H), 1.29-1.14 (m, 4H) | 370.1 | 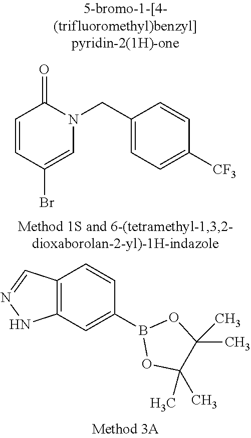 [SMILES: O=c1ccc(Br)cn1Cc1ccc(C(F)(F)F)cc1 ; CC1(C)OB(c2ccc3cn[nH]c3c2)OC1(C)C] |
| 1U | 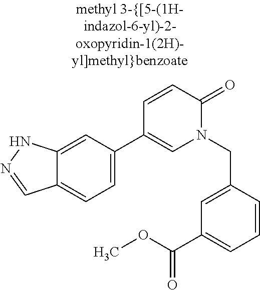 [SMILES: COC(=O)c1cccc(Cn2cc(-c3ccc4cn[nH]c4c3)ccc2=O)c1] | (400 MHz, DMSO) δ 13.14 (s, 1H), 8.42 (d, J = 2.1 Hz, 1H), 8.08 (s, 1H), 8.01 (s, 1H), 7.95 (dd, J = 9.4, 2.2 Hz, 1H), 7.89 (d, J = 7.6 Hz, 1H), 7.81 (d, J = 8.4 Hz, 1H), 7.70 (d, J = 7.5 Hz, 1H), 7.66 (s, 1H), 7.52 (t, J = 7.7 Hz, 1H), 7.34 (d, J = 8.4 Hz, 1H), 6.57 (d, J = 9.4 Hz, 1H), 5.28 (s, 2H), 3.85 (s, 3H) | 360.1 | 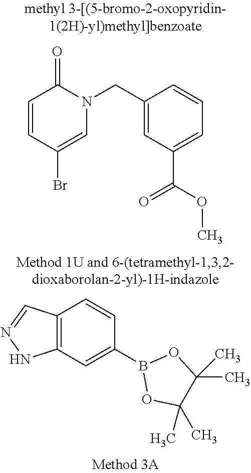 [SMILES: COC(=O)c1cccc(Cn2cc(Br)ccc2=O)c1 ; CC1(C)OB(c2ccc3cn[nH]c3c2)OC1(C)C] |
| 1W | 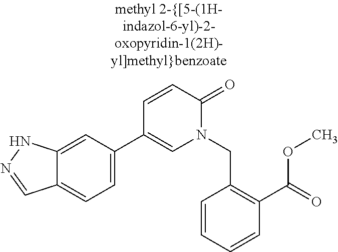 [SMILES: COC(=O)c1ccccc1Cn1cc(-c2ccc3cn[nH]c3c2)ccc1=O] | (400 MHz, DMSO) δ 13.13 (s, 1H), 8.30 (d, J = 2.6 Hz, 1H), 8.07 (s, 1H), 8.02 (dd, J = 9.5, 2.7 Hz, 1H), 7.95 (dd, J = 7.8, 1.2 Hz, 1H), 7.80 (d, J = 8.5 Hz, 1H), 7.67 (s, 1H), 7.57 (td, J = 7.7, 1.3 Hz, 1H), 7.43 (t, J = 7.6 Hz, 1H), 7.34 (dd, J = 8.5, 1.4 Hz, 1H), 6.93 (d, J = 7.8 Hz, 1H), 6.60 (d, J = 9.5 Hz, 1H), 5.56 (s, 2H), 3.91 (s, 3H) | 360.1 | 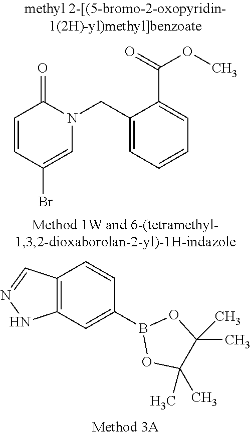 [SMILES: COC(=O)c1ccccc1Cn1cc(Br)ccc1=O ; CC1(C)OB(c2ccc3cn[nH]c3c2)OC1(C)C] |
| 1X | 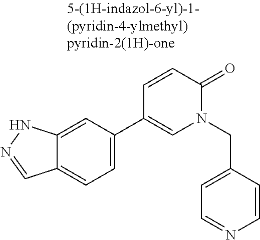 [SMILES: O=c1ccc(-c2ccc3cn[nH]c3c2)cn1Cc1ccncc1] | | 303.0 | 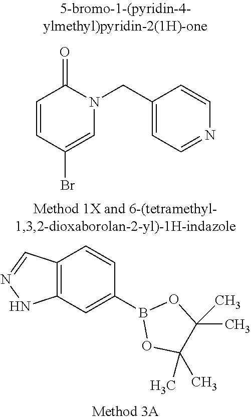 [SMILES: O=c1ccc(Br)cn1Cc1ccncc1 ; CC1(C)OB(c2ccc3cn[nH]c3c2)OC1(C)C] |
| 1Y | 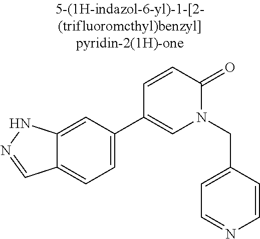 [SMILES: O=c1ccc(-c2ccc3cn[nH]c3c2)cn1Cc1ccncc1] | (400 MHz, DMSO) δ 13.14 (s, 1H), 8.31 (d, J = 2.6 Hz, 1H), 8.06 (dd, J = 9.6, 2.8 Hz, 2H), 7.81 (d, J = 8.3 Hz, 2H), 7.69 (s, 1H), 7.64 (t, J = 7.6 Hz, 1H), 7.51 (t, J = 7.8 Hz, 1H), 7.36 (dd, J = 8.5, 1.4 Hz, 1H), 6.92 (d, J = 7.9 Hz, 1H), 6.63 (d, J = 9.5 Hz, 1H), 5.44 (s, 2H) | 367.9 | 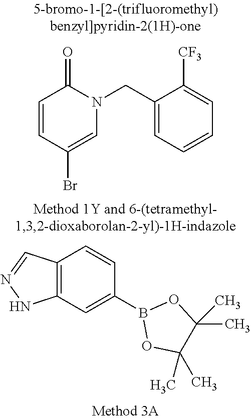 [SMILES: O=c1ccc(Br)cn1Cc1ccccc1C(F)(F)F ; CC1(C)OB(c2ccc3cn[nH]c3c2)OC1(C)C] |
| 1Z | 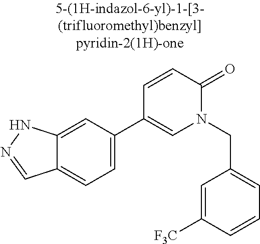 [SMILES: O=c1ccc(-c2ccc3cn[nH]c3c2)cn1Cc1cccc(C(F)(F)F)c1] | (400 MHz, DMSO) δ 13.14 (s, 1H), 8.44 (d, J = 2.7 Hz, 1H), 8.07 (s, 1H), 7.95 (dd, J = 9.5, 2.7 Hz, 1H), 7.81 (d, J = 8.7 Hz, 2H), 7.71-7.65 (m, 3H), 7.61 (d, J = 7.8 Hz, 1H), 7.35 (dd, J = 8.5, 1.5 Hz, 1H), 6.57 (d, J = 9.5 Hz, 1H), 5.29 (s, 2H) | 370.1 | 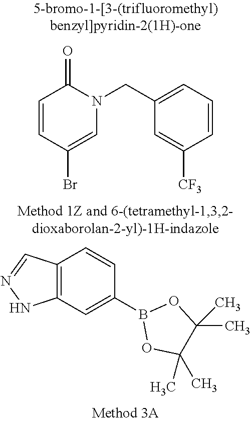 [SMILES: O=c1ccc(Br)cn1Cc1cccc(C(F)(F)F)c1 ; CC1(C)OB(c2ccc3cn[nH]c3c2)OC1(C)C] |
| 1AA | 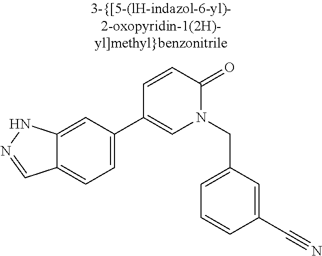 [SMILES: N#Cc1cccc(Cn2cc(-c3ccc4cn[nH]c4c3)ccc2=O)c1] | (400 MHz, DMSO) δ 13.14 (s, 1H), 8.40 (d, J = 2.5 Hz, 1H), 8.08 (s, 1H), 7.95 (dd, J = 9.5, 2.7 Hz, 1H), 7.88 (s, 1H), 7.81 (d, J = 8.4 Hz, 1H), 7.78 (d, J = 7.8 Hz, 1H), 7.74 (d, J = 8.0 Hz, 1H), 7.67 (s, 1H), 7.58 (t, J = 7.8 Hz, 1H), 7.36 (d, J = 9.7 Hz, 1H), 6.56 (d, J = 9.5 Hz, 1H), 5.24 (s, 2H) | 327.0 | 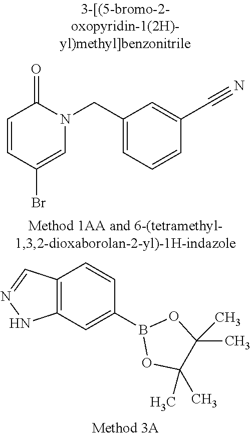 [SMILES: N#Cc1cccc(Cn2cc(Br)ccc2=O)c1 ; CC1(C)OB(c2ccc3cn[nH]c3c2)OC1(C)C] |
| 1AB | 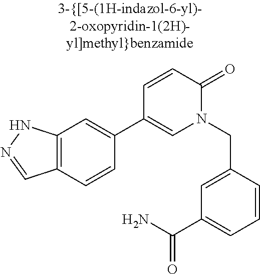 [SMILES: NC(=O)c1cccc(Cn2cc(-c3ccc4cn[nH]c4c3)ccc2=O)c1] | (400 MHz, DMSO) δ 13.14 (s, 1H), 8.37 (d, J = 2.6 Hz, 1H), 8.07 (s, 1H), 8.00 (s, 1H), 7.94 (dd, J = 9.5, 2.7 Hz, 1H), 7.89 (s, 1H), 7.80 (t, J = 8.7 Hz, 2H), 7.66 (s, 1H), 7.52 (d, J = 7.9 Hz, 1H), 7.44 (d, J = 7.7 Hz, 1H), 7.39 (s, 1H), 7.35 (dd, J = 8.5, 1.5 Hz, 1H), 6.57 (d, J = 9.5 Hz, 1H), 5.25 (s, 2H) | 345.1 | 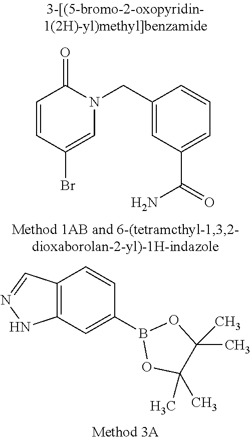 [SMILES: NC(=O)c1cccc(Cn2cc(Br)ccc2=O)c1 ; CC1(C)OB(c2ccc3cn[nH]c3c2)OC1(C)C] |
| 1AC | 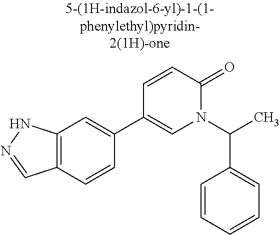 [SMILES: CC(c1ccccc1)n1cc(-c2ccc3cn[nH]c3c2)ccc1=O] | (400 MHz, DMSO) δ 13.10 (s, 1H), 8.06 (s, 1H), 7.91 (d, J = 2.5 Hz, 1H), 7.87 (dd, J = 9.4, 2.6 Hz, 1H), 7.78 (d, J = 8.4 Hz, 1H), 7.58 (s, 1H), 7.44-7.35 (m, 4H), 7.33- 7.23 (m, 2H), 6.57 (d, J = 9.4 Hz, 1H), 6.27 (q, J = 7.2 Hz, 1H), 1.82 (d, J = 7.2 Hz, 3H) | 316.2 | 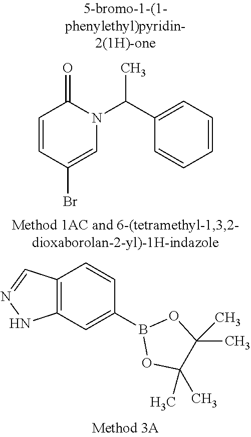 [SMILES: CC(c1ccccc1)n1cc(Br)ccc1=O ; CC1(C)OB(c2ccc3cn[nH]c3c2)OC1(C)C] |
| 1AD | 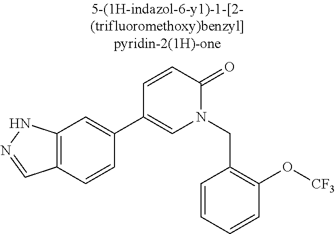 [SMILES: O=c1ccc(-c2ccc3cn[nH]c3c2)cn1Cc1ccccc1OC(F)(F)F] | (400 MHz, DMSO) δ 13.14 (s, 1H), 8.28 (d, J = 2.6 Hz, 1H), 8.07 (s, 1H), 7.99 (dd, J = 9.5, 2.7 Hz, 1H), 7.81 (d, J = 8.5 Hz, 1H), 7.67 (s, 1H), 7.48-7.40 (m, 2H), 7.38-7.32 (m, 2H), 7.16 (d, J = 7.4 Hz, 1H), 6.58 (d, J = 9.5 Hz, 1H), 5.29 (s, 2H) | 386.6 | 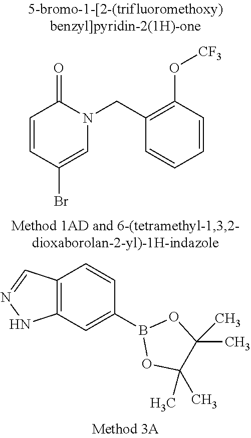 [SMILES: O=c1ccc(Br)cn1Cc1ccccc1OC(F)(F)F ; CC1(C)OB(c2ccc3cn[nH]c3c2)OC1(C)C] |
| 1AE | 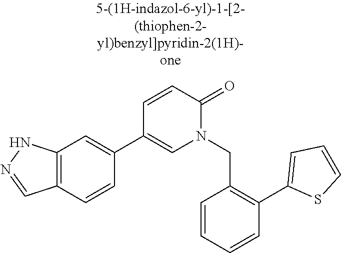 [SMILES: O=c1ccc(-c2ccc3cn[nH]c3c2)cn1Cc1ccccc1-c1cccs1] | (400 MHz, DMSO) δ 13.12 (s, 1H), 8.06 (s, 1H), 8.04 (d, J = 2.7 Hz, 1H), 7.97 (dd, J = 9.4, 2.7 Hz, 1H), 7.79 (d, J = 8.5 Hz, 1H), 7.70 (dd, J = 5.1, 1.0 Hz, 1H), 7.61 (s, 1H), 7.45 (dd, J = 5.7, 3.3 Hz, 1H), 7.38-7.35 (m, 2H), 7.32 (dd, J = 3.5, 1.0 Hz, 1H), 7.28 (dd, J = 8.5, 1.4 Hz, 1H), 7.22 (dd, J = 5.1, 3.5 Hz, 1H), 6.98- 6.95 (m, 1H), 6.56 (d, J = 9.4 Hz, 1H), 5.34 (s, 2H) | 384.2 | 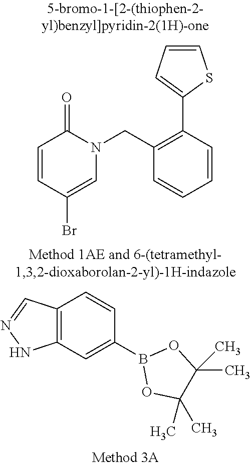 [SMILES: O=c1ccc(Br)cn1Cc1ccccc1-c1cccs1 ; CC1(C)OB(c2ccc3cn[nH]c3c2)OC1(C)C] |
| 1AG | 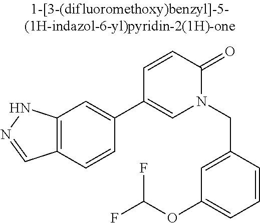 [SMILES: O=c1ccc(-c2ccc3cn[nH]c3c2)cn1Cc1cccc(OC(F)F)c1] | (400 MHz, DMSO) δ 13.14 (s, 1H), 8.36 (d, J = 2.6 Hz, 1H), 8.07 (d, J = 0.9 Hz, 1H), 7.94 (dd, J = 9.5, 2.7 Hz, 1H), 7.81 (d, J = 8.5 Hz, 1H), 7.66 (s, 1H), 7.42-7.39 (m, 1H), 7.34 (dd, J = 8.5, 1.5 Hz, 1H), 7.26 (d, J = 7.8 Hz, 1H), 7.22 (d, J = 2.4 Hz, 2H), 7.11 (dd, J = 8.1, 2.2 Hz, 1H), 6.57 (d, J = 9.5 Hz, 1H), 5.22 (s, 2H) | 368.2 | 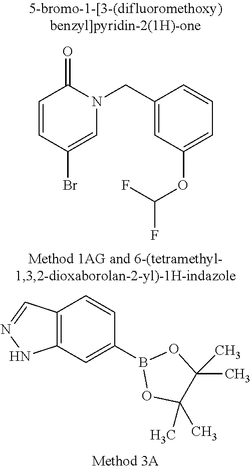 [SMILES: O=c1ccc(Br)cn1Cc1cccc(OC(F)F)c1 ; CC1(C)OB(c2ccc3cn[nH]c3c2)OC1(C)C] |
| 1AH | 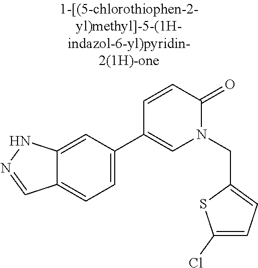 [SMILES: O=c1ccc(-c2ccc3cn[nH]c3c2)cn1Cc1ccc(Cl)s1] | (400 MHz, DMSO) δ 13.15 (s, 1H), 8.39 (d, J = 2.6 Hz, 1H), 8.08 (s, 1H), 7.93 (dd, J = 9.5, 2.7 Hz, 1H), 7.82 (d, J = 8.4 Hz, 1H), 7.64 (s, 1H), 7.32 (dd, J = 8.4, 1.4 Hz, 1H), 7.17 (d, J = 3.8 Hz, 1H), 6.99 (d, J = 3.8 Hz, 1H), 6.57 (d, J = 9.5 Hz, 1H), 5.27 (s, 2H) | 342.2 | 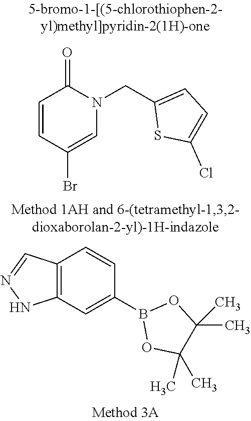 [SMILES: O=c1ccc(Br)cn1Cc1ccc(Cl)s1 ; CC1(C)OB(c2ccc3cn[nH]c3c2)OC1(C)C] |
| 1AI | 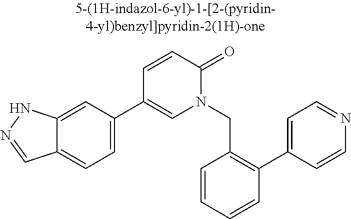 [SMILES: O=c1ccc(-c2ccc3cn[nH]c3c2)cn1Cc1ccccc1-c1ccncc1] | (300 MHz, DMSO) δ 13.10 (s, 2H), 8.66 (dd, J = 4.4, 1.6 Hz, 3H), 8.03 (d, J = 2.9 Hz, 3H), 7.97-7.87 (m, 2H), 7.77 (d, J = 8.4 Hz, 2H), 7.59 (s, 2H), 7.49 (dd, J = 4.4, 1.6 Hz, 4H), 7.39 (dd, J = 5.7, 3.3 Hz, 4H), 7.32- 7.21 (m, 4H), 7.01 (dd, J = 5.3, 3.7 Hz, 2H), 6.49 (d, J = 9.5 Hz, 2H), 5.17 (s, 3H) | 379.2 | 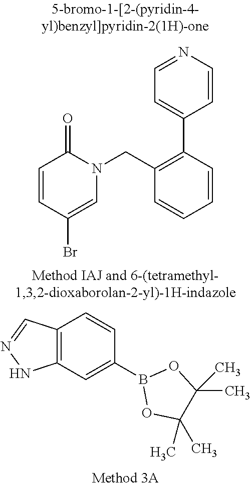 [SMILES: O=c1ccc(Br)cn1Cc1ccccc1-c1ccncc1 ; CC1(C)OB(c2ccc3cn[nH]c3c2)OC1(C)C] |
| 1AJ | 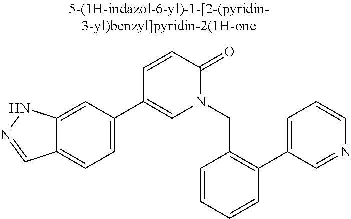 [SMILES: O=c1ccc(-c2ccc3cn[nH]c3c2)cn1Cc1ccccc1-c1cccnc1] | (300 MHz, DMSO) δ 13.10 (s, 1H), 8.70-8.49 (m, 2H), 8.08-7.97 (m, 2H), 7.91 (dd, J = 11.8, 5.1 Hz, 2H), 7.77 (d, J = 8.4 Hz, 1H), 7.58 (s, 1H), 7.49 (dd, J = 7.7, 4.7 Hz, 1H), 7.38 (dd, J = 5.7, 3.4 Hz, 2H), 7.33-7.19 (m, 2H), 7.07-6.97 (m, 1H), 6.48 (d, J = 9.5 Hz, 1H), 5.15 (s, 2H) | 379.3 | 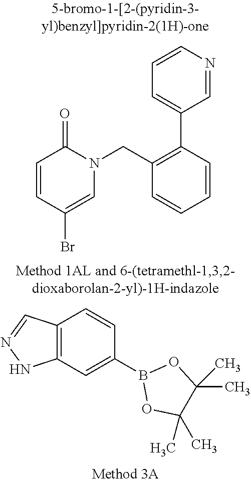 [SMILES: O=c1ccc(Br)cn1Cc1ccccc1-c1cccnc1 ; CC1(C)OB(c2ccc3cn[nH]c3c2)OC1(C)C] |
| 1AK | 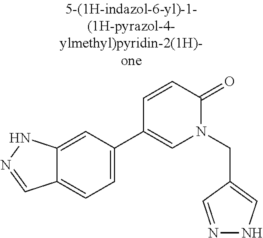 [SMILES: O=c1ccc(-c2ccc3cn[nH]c3c2)cn1Cc1cn[nH]c1] | (300 MHz, DMSO) δ 13.08 (bs, 1H), 8.25 (d, J = 2.5 Hz, 1H), 8.04 (d, J = 0.9 Hz, 1H), 7.84 (dd, J = 9.4, 2.7 Hz, 1H), 7.78 (dd, J = 8.5, 0.7 Hz, 1H), 7.67 (s, 2H), 7.59 (s, 1H), 7.29 (dd, J = 8.5, 1.5 Hz, 1H), 6.49 (d, J = 9.4 Hz, 1H), 5.02 (s, 2H) | 292.3 | 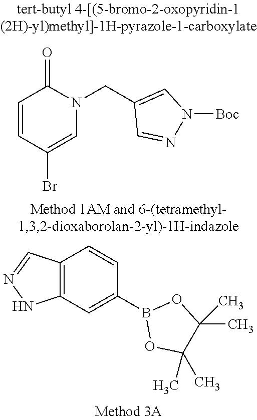 [SMILES: CC(C)(C)OC(=O)n1cc(Cn2cc(Br)ccc2=O)cn1 ; CC1(C)OB(c2ccc3cn[nH]c3c2)OC1(C)C] |
| 1AM | 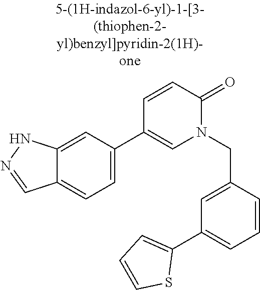 [SMILES: O=c1ccc(-c2ccc3cn[nH]c3c2)cn1Cc1cccc(-c2cccs2)c1] | (400 MHz, DMSO-d6) δ 13.12 (s, 1H), 8.39 (d, J = 2.7 Hz, 1H), 8.07 (s, 1H), 7.94 (dd, J = 9.5, 2.6 Hz, 1H), 7.81 (d, J = 8.4 Hz, 1H), 7.71 (s, 1H), 7.66 (s, 1H), 7.60 (d, J = 7.7 Hz, 1H), 7.57-7.52 (m, 1H), 7.50-7.46 (m, 1H), 7.40 (t, J = 7.7 Hz, 1H), 7.37- 7.33 (m, 1H), 7.30 (d, J = 7.7 Hz, 1H), 7.17-7.11 (m, 1H), 6.58 (d, J = 9.4 Hz, 1H), 5.26 (s, 2H). | 384.3 | 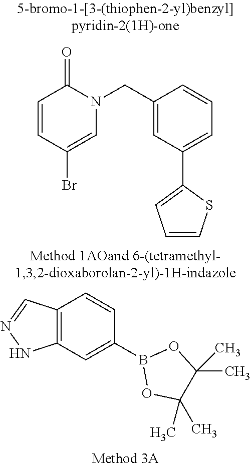 [SMILES: O=c1ccc(Br)cn1Cc1cccc(-c2cccs2)c1 ; CC1(C)OB(c2ccc3cn[nH]c3c2)OC1(C)C] |
| 1AN | 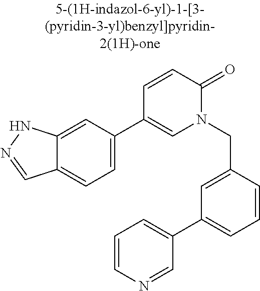 [SMILES: O=c1ccc(-c2ccc3cn[nH]c3c2)cn1Cc1cccc(-c2cccnc2)c1] | (400 MHz, DMSO) δ 13.13 (s, 1H), 8.89 (s, 1H), 8.60 (s, 1H), 8.41 (d, J = 2.4 Hz, 1H), 8.06 (d, J = 13.2 Hz, 2H), 7.94 (dd, J = 9.5, 2.5 Hz, 1H), 7.81 (d, J = 8.1 Hz, 2H), 7.66 (s, 2H), 7.50 (t, J = 7.5 Hz, 2H), 7.44 (d, J = 7.7 Hz, 1H), 7.35 (d, J = 8.5 Hz, 1H), 6.57 (d, J = 9.4 Hz, 1H), 5.29 (s, 2H) | 379.2 | 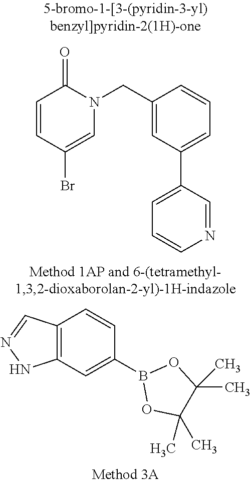 [SMILES: O=c1ccc(Br)cn1Cc1cccc(-c2cccnc2)c1 ; CC1(C)OB(c2ccc3cn[nH]c3c2)OC1(C)C] |
| 1AO | 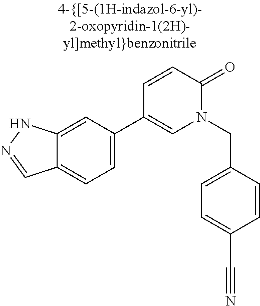 [SMILES: N#Cc1ccc(Cn2cc(-c3ccc4cn[nH]c4c3)ccc2=O)cc1] | (400 MHz, DMSO) δ 13.14 (s, 1H), 8.39 (d, J = 2.6 Hz, 1H), 8.08 (s, 1H), 7.97 (dd, J = 9.5, 2.7 Hz, 1H), 7.85 (s, 1H), 7.82 (d, J = 9.9 Hz, 2H), 7.67 (s, 1H), 7.54 (d, J = 8.3 Hz, 2H), 7.35 (dd, J = 8.5, 1.2 Hz, 1H), 6.58 (d, J = 9.5 Hz, 1H), 5.29 (s, 2H) | 327.2 | 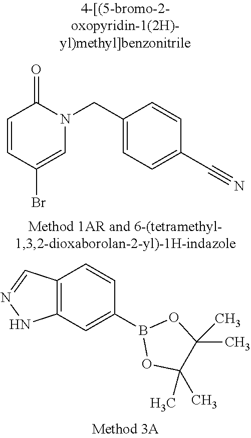 [SMILES: N#Cc1ccc(Cn2cc(Br)ccc2=O)cc1 ; CC1(C)OB(c2ccc3cn[nH]c3c2)OC1(C)C] |
| 1AS | 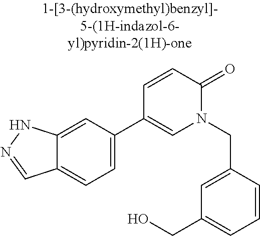 [SMILES: O=c1ccc(-c2ccc3cn[nH]c3c2)cn1Cc1cccc(CO)c1] | | 332.2 | 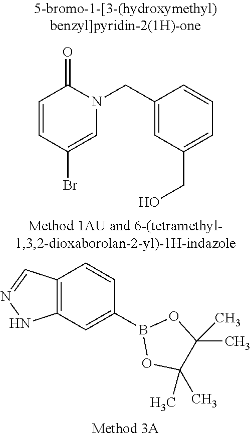 [SMILES: O=c1ccc(Br)cn1Cc1cccc(CO)c1 ; CC1(C)OB(c2ccc3cn[nH]c3c2)OC1(C)C] |
| 1AT | 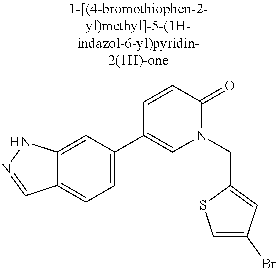 [SMILES: O=c1ccc(-c2ccc3cn[nH]c3c2)cn1Cc1cc(Br)cs1] | (400 MHz, DMSO) δ 13.15 (s, 1H), 8.40 (d, J = 2.6 Hz, 1H), 8.08 (s, 1H), 7.93 (dd, J = 9.5, 2.7 Hz, 1H), 7.82 (d, J = 8.4 Hz, 1H), 7.65 (s, 1H), 7.60 (d, J = 1.6 Hz, 1H), 7.33 (dd, J = 8.5, 1.5 Hz, 1H), 7.29 (d, J = 1.5 Hz, 1H), 6.57 (d, J = 9.5 Hz. 1H), 5.32 (s, 2H) | 386.4 | 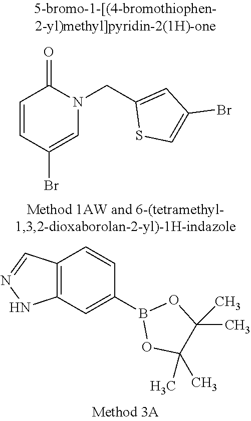 [SMILES: O=c1ccc(Br)cn1Cc1cc(Br)cs1 ; CC1(C)OB(c2ccc3cn[nH]c3c2)OC1(C)C] |
| 1AU | 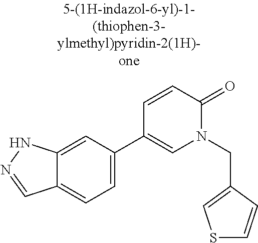 [SMILES: O=c1ccc(-c2ccc3cn[nH]c3c2)cn1Cc1ccsc1] | (300 MHz, DMSO) δ 13.09 (s, 1H), 8.29 (d, J = 2.3 Hz, 1H), 8.04 (s, 1H), 7.88 (dd, J = 9.5, 2.7 Hz, 1H), 7.78 (d, J = 8.4 Hz, 1H), 7.61 (s, 1H), 7.49 (dd, J = 4.1, 2.8 Hz, 2H), 7.30 (dd, J = 8.5, 1.5 Hz, 1H), 7.16 (dd, J = 3.8, 2.5 Hz, 1H), 6.52 (d, J = 9.5 Hz, 1H), 5.16 (s, 2H) | 308.2 | 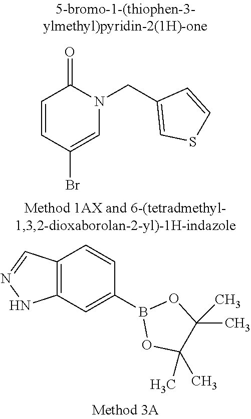 [SMILES: O=c1ccc(Br)cn1Cc1ccsc1 ; CC1(C)OB(c2ccc3cn[nH]c3c2)OC1(C)C] |
| 1AW | 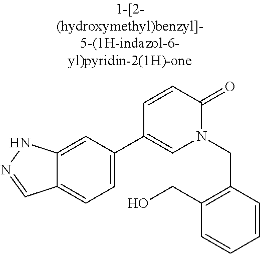 [SMILES: O=c1ccc(-c2ccc3cn[nH]c3c2)cn1Cc1ccccc1CO] | (400 MHz, DMSO) δ 13.12 (s, 1H), 8.23 (d, J = 2.6 Hz, 1H), 8.07 (s, 1H), 7.98 (dd, J = 9.5, 2.7 Hz, 1H), 7.80 (d, J = 8.4 Hz, 1H), 7.64 (s, 1H), 7.44 (d, J = 7.2 Hz, 1H), 7.32 (dd, J = 8.5, 1.4 Hz, 1H), 7.25 (ddd, J = 9.1, 7.3, 1.4 Hz, 2H), 6.94 (d, J = 7.3 Hz, 1H), 6.60 (d, J = 9.4 Hz, 1H), 5.32 (t, J = 5.4 Hz, 1H), 5.27 (s, 2H), 4.67 (d, J = 5.4 Hz, 2H) | 332.1 | 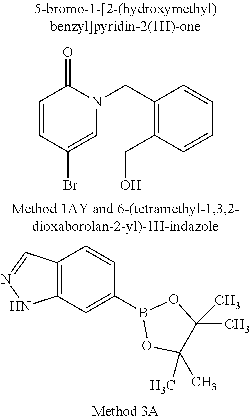 [SMILES: O=c1ccc(Br)cn1Cc1ccccc1CO ; CC1(C)OB(c2ccc3cn[nH]c3c2)OC1(C)C] |
| 1AX | 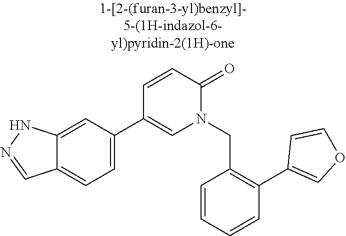 [SMILES: O=c1ccc(-c2ccc3cn[nH]c3c2)cn1Cc1ccccc1-c1ccoc1] | | 368.2 | 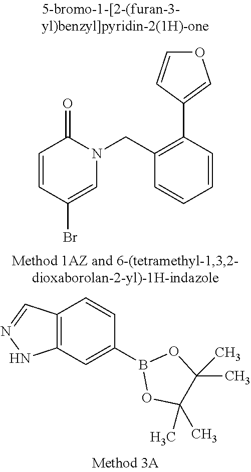 [SMILES: O=c1ccc(Br)cn1Cc1ccccc1-c1ccoc1 ; CC1(C)OB(c2ccc3cn[nH]c3c2)OC1(C)C] |
| 1AY | 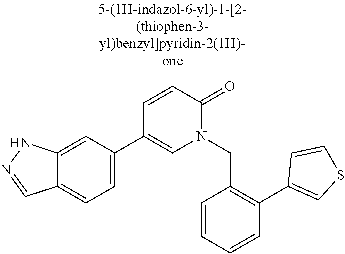 [SMILES: O=c1ccc(-c2ccc3cn[nH]c3c2)cn1Cc1ccccc1-c1ccsc1] | (300 MHz, DMSO) δ 13.09 (s, 1H), 8.04 (s, 1H), 7.97-7.90 (m, 2H), 7.77 (d, J = 8.4 Hz, 1H), 7.69 (dd, J = 4.9, 2.9 Hz, 1H), 7.64 (dd, J = 2.9, 1.3 Hz, 1H), 7.57 (s, 1H), 7.35-7.31 (m, 3H), 7.30-7.28 (m, 1H), 7.25 (dd, J = 8.5, 1.4 Hz, 1H), 6.96-6.91 (m, 1H), 6.52 (d, J = 9.1 Hz, 1H), 5.25 (s, 2H) | 384.1 | 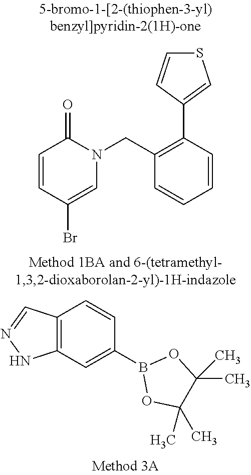 [SMILES: O=c1ccc(Br)cn1Cc1ccccc1-c1ccsc1 ; CC1(C)OB(c2ccc3cn[nH]c3c2)OC1(C)C] |
| 1AZ | 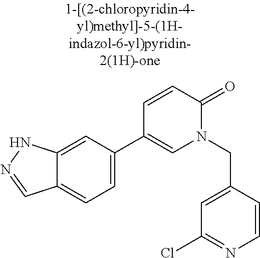 [SMILES: O=c1ccc(-c2ccc3cn[nH]c3c2)cn1Cc1ccnc(Cl)c1] | (400 MHz, DMSO) δ 13.15 (s, 1H), 8.39 (d, J = 3.8 Hz, 2H), 8.08 (s, 1H), 7.82 (d, J = 8.4 Hz, 1H), 7.69 (s, 1H), 7.44 (s, 2H), 7.34 (dd, J = 19.3, 6.7 Hz, 2H), 6.59 (d, J = 9.4 Hz, 1H), 5.25 (s, 2H) | 336.8 | 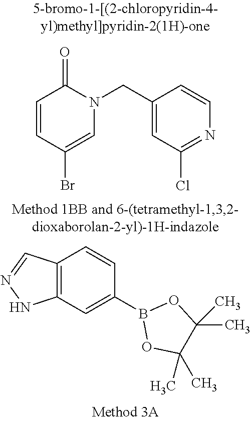 [SMILES: O=c1ccc(Br)cn1Cc1ccnc(Cl)c1 ; CC1(C)OB(c2ccc3cn[nH]c3c2)OC1(C)C] |
| 1BA | 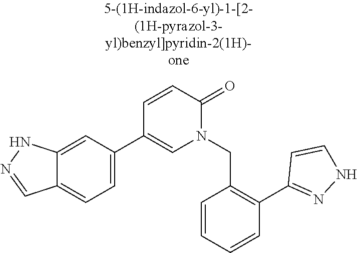 [SMILES: O=c1ccc(-c2ccc3cn[nH]c3c2)cn1Cc1ccccc1-c1cc[nH]n1] | (400 MHz, DMSO) δ 13.11 (s, 2H), 8.18 (s, 1H), 8.06 (s, 1H), 7.97 (d, J = 9.6 Hz, 1H), 7.92 (s, 1H), 7.78 (d, J = 8.4 Hz, 1H), 7.66 (d, J = 7.5 Hz, 1H), 7.60 (s, 1H), 7.40-7.25 (m, 3H), 6.96 (d, J = 7.6 Hz, 1H), 6.69 (s, 1H), 6.59 (d, J = 9.5 Hz, 1H), 5.55 (s, 2H) | 368.0 | 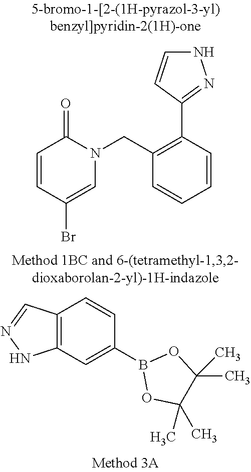 [SMILES: O=c1ccc(Br)cn1Cc1ccccc1-c1cc[nH]n1 ; CC1(C)OB(c2ccc3cn[nH]c3c2)OC1(C)C] |
| 1BB | 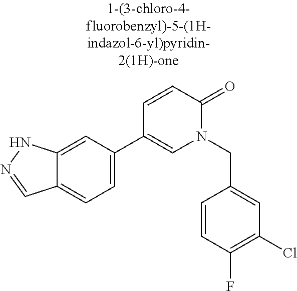 [SMILES: O=c1ccc(-c2ccc3cn[nH]c3c2)cn1Cc1ccc(F)c(Cl)c1] | (400 MHz, DMSO) δ 13.14 (s, 1H), 8.40 (d, J = 2.6 Hz, 1H), 8.08 (s, 1H), 7.94 (dd, J = 9.5, 2.7 Hz, 1H), 7.82 (d, J = 8.5 Hz, 1H), 7.71-7.64 (m, 2H), 7.49-7.37 (m, 2H), 7.35 (dd, J = 8.5, 1.3 Hz, 1H), 6.56 (d, J = 9.4 Hz, 1H), 5.18 (s, 2H) | 354.0 | 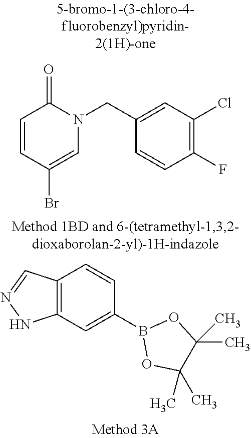 [SMILES: O=c1ccc(Br)cn1Cc1ccc(F)c(Cl)c1 ; CC1(C)OB(c2ccc3cn[nH]c3c2)OC1(C)C] |
| 1BC | 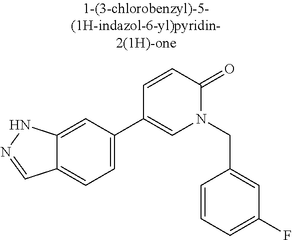 [SMILES: O=c1ccc(-c2ccc3cn[nH]c3c2)cn1Cc1cccc(F)c1] | (400 MHz, DMSO) δ 13.14 (s, 1H), 8.37 (d, J = 2.6 Hz, 1H), 8.07 (s, 1H), 7.95 (dd, J = 9.5, 2.7 Hz, 1H), 7.81 (d, J = 8.4 Hz, 1H), 7.66 (s, 1H), 7.44-7.37 (m, 1H), 7.36-7.32 (m, 1H), 7.23 (d, J = 7.9 Hz, 2H), 7.14 (t, J = 8.2 Hz, 1H), 6.57 (d, J = 9.5 Hz, 1H), 5.22 (s, 2H) | 320.0 | 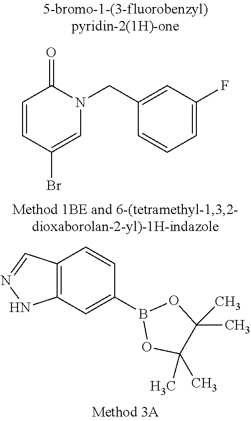 [SMILES: O=c1ccc(Br)cn1Cc1cccc(F)c1 ; CC1(C)OB(c2ccc3cn[nH]c3c2)OC1(C)C] |
| 1BD | 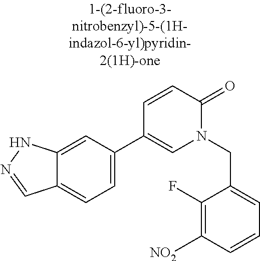 [SMILES: O=c1ccc(-c2ccc3cn[nH]c3c2)cn1Cc1cccc([N+](=O)[O-])c1F] | (400 MHz, DMSO) δ 13.16 (s, 1H), 8.38 (d, J = 2.4 Hz, 1H), 8.13- 8.06 (m, 2H), 7.99 (dd, J = 9.5, 2.7 Hz, 1H), 7.83 (d, J = 8.4 Hz, 1H), 7.68 (s, 1H), 7.63 (t, J = 6.4 Hz, 1H), 7.43 (t, J = 8.0 Hz, 1H), 7.36 (dd, J = 8.5, 1.3 Hz, 1H), 6.57 (d, J = 9.5 Hz, 1H), 5.34 (s, 2H) | 365.0 | 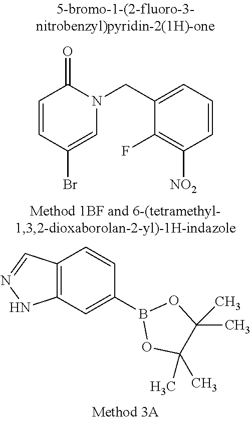 [SMILES: O=c1ccc(Br)cn1Cc1cccc([N+](=O)[O-])c1F ; CC1(C)OB(c2ccc3cn[nH]c3c2)OC1(C)C] |
| 1BE | 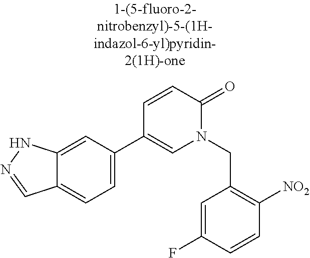 [SMILES: O=c1ccc(-c2ccc3cn[nH]c3c2)cn1Cc1cc(F)ccc1[N+](=O)[O-]] | (400 MHz, DMSO) δ 13.16 (s, 1H), 8.34 (d, J = 2.6 Hz, 1H), 8.27 (dd, J = 9.1, 5.2 Hz, 1H), 8.08 (s, 1H), 8.05 (dd, J = 9.5, 2.7 Hz, 1H), 7.82 (d, J = 8.4 Hz, 1H), 7.69 (s, 1H), 7.49- 7.42 (m, 1H), 7.36 (dd, J = 8.5, 1.4 Hz, 1H), 6.84 (dd, J = 9.5, 2.7 Hz, 1H), 6.61 (d, J = 9.5 Hz, 1H), 5.53 (s, 2H) | 365.0 | 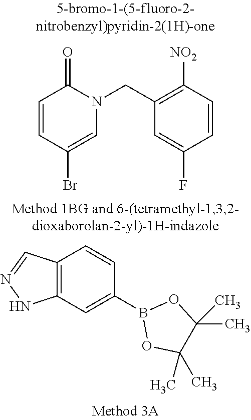 [SMILES: O=c1ccc(Br)cn1Cc1cc(F)ccc1[N+](=O)[O-] ; CC1(C)OB(c2ccc3cn[nH]c3c2)OC1(C)C] |
| 1BF | 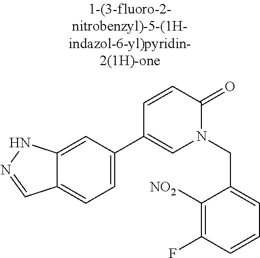 [SMILES: O=c1ccc(-c2ccc3cn[nH]c3c2)cn1Cc1cccc(F)c1[N+](=O)[O-]] | (400 MHz, DMSO) δ 13.16 (s, 1H), 8.38 (d, J = 2.6 Hz, 1H), 8.08 (s, 1H), 8.01 (dd, J = 9.5, 2.6 Hz, 1H), 7.82 (d, J = 8.5 Hz, 1H), 7.73- 7.65 (m, 2H), 7.59-7.53 (m, 1H), 7.35 (d, J = 8.5 Hz, 1H), 7.10 (d, J = 7.9 Hz, 1H), 6.56 (d, J = 9.5 Hz, 1H), 5.33 (s, 2H) | 365.0 | 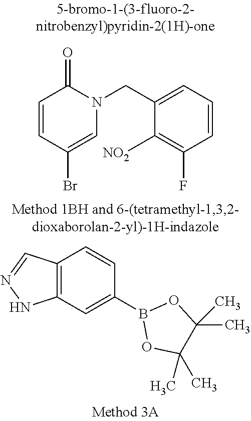 [SMILES: O=c1ccc(Br)cn1Cc1cccc(F)c1[N+](=O)[O-] ; CC1(C)OB(c2ccc3cn[nH]c3c2)OC1(C)C] |
| 1BG | 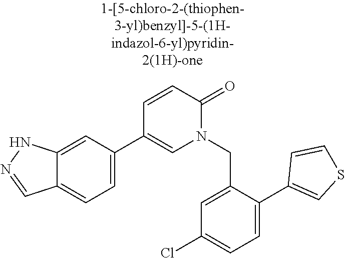 [SMILES: O=c1ccc(-c2ccc3cn[nH]c3c2)cn1Cc1cc(Cl)ccc1-c1ccsc1] | | 418.0 | 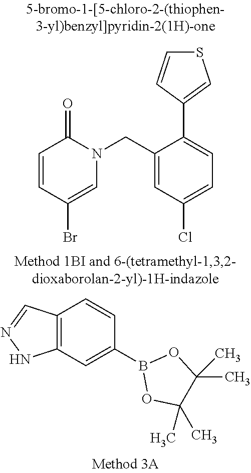 [SMILES: O=c1ccc(Br)cn1Cc1cc(Cl)ccc1-c1ccsc1 ; CC1(C)OB(c2ccc3cn[nH]c3c2)OC1(C)C] |
| 1BH | 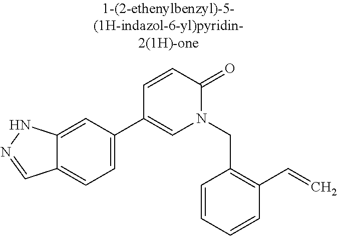 [SMILES: C=Cc1ccccc1Cn1cc(-c2ccc3cn[nH]c3c2)ccc1=O] | (400 MHz, DMSO) δ 13.12 (s, 1H), 8.14 (d, J = 2.5 Hz, 1H), 8.06 (s, 1H), 7.97 (dd, J = 9.5, 2.5 Hz, 1H), 7.80 (d, J = 8.4 Hz, 1H), 7.61 (d, J = 9.1 Hz, 2H), 7.35- 7.25 (m, 3H), 7.20 (dd, J = 17.3, 11.0 Hz, 1H), 7.01 (d, J = 7.5 Hz, 1H), 6.60 (d, J = 9.4 Hz, 1H), 5.78 (d, J = 17.4 Hz, 1H), 5.41 (d, J = 11.1 Hz, 1H), 5.32 (s, 2H) | 328.2 | 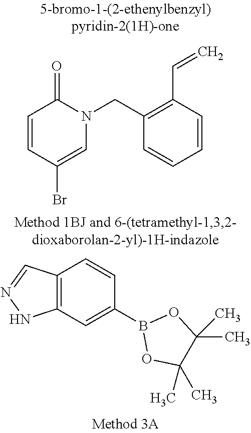 [SMILES: C=Cc1ccccc1Cn1cc(Br)ccc1=O ; CC1(C)OB(c2ccc3cn[nH]c3c2)OC1(C)C] |
| 1BI | 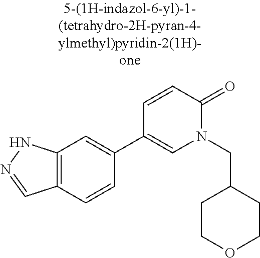 [SMILES: O=c1ccc(-c2ccc3cn[nH]c3c2)cn1CC1CCOCC1] | | 310.1 | 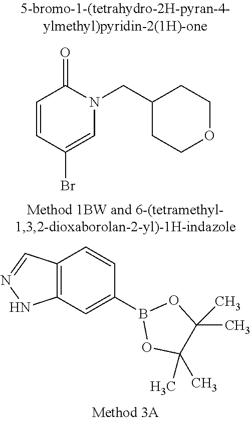 [SMILES: O=c1ccc(Br)cn1CC1CCOCC1 ; CC1(C)OB(c2ccc3cn[nH]c3c2)OC1(C)C] |
| 1BK | 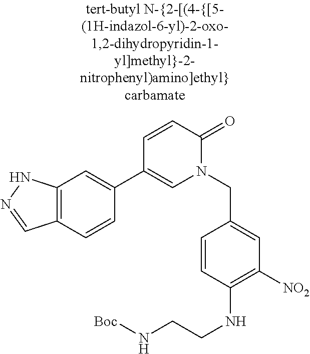 [SMILES: CC(C)(C)OC(=O)NCCNc1ccc(Cn2cc(-c3ccc4cn[nH]c4c3)ccc2=O)cc1[N+](=O)[O-]] | | | 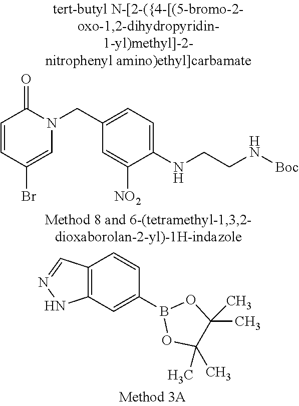 [SMILES: CC(C)(C)OC(=O)NCCNc1ccc(Cn2cc(Br)ccc2=O)cc1[N+](=O)[O-] ; CC1(C)OB(c2ccc3cn[nH]c3c2)OC1(C)C] |
| 1BN | 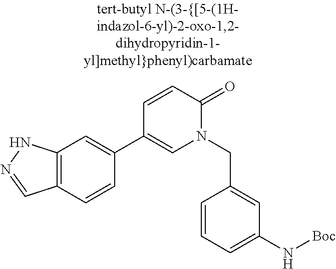 [SMILES: CC(C)(C)OC(=O)Nc1cccc(Cn2cc(-c3ccc4cn[nH]c4c3)ccc2=O)c1] | | | 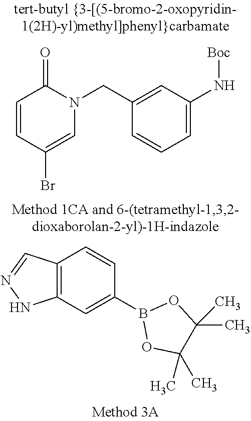 [SMILES: CC(C)(C)OC(=O)Nc1cccc(Cn2cc(Br)ccc2=O)c1 ; CC1(C)OB(c2ccc3cn[nH]c3c2)OC1(C)C] |
| 1BO | 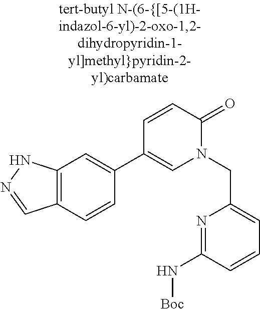 [SMILES: CC(C)(C)OC(=O)Nc1cccc(Cn2cc(-c3ccc4cn[nH]c4c3)ccc2=O)n1] | | | 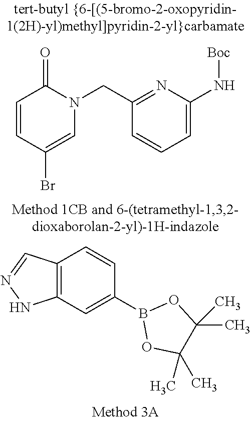 [SMILES: CC(C)(C)OC(=O)Nc1cccc(Cn2cc(Br)ccc2=O)n1 ; CC1(C)OB(c2ccc3cn[nH]c3c2)OC1(C)C] |
| 1BP | 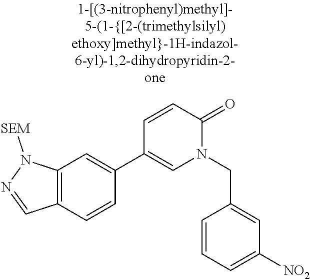 [SMILES: C[Si](C)(C)CCOCn1ncc2ccc(-c3ccc(=O)n(Cc4cccc([N+](=O)[O-])c4)c3)cc21] | | | 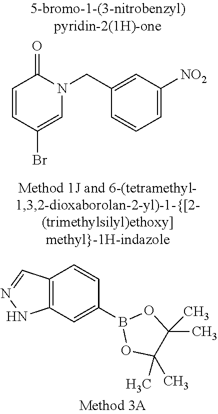 [SMILES: O=c1ccc(Br)cn1Cc1cccc([N+](=O)[O-])c1 ; CC1(C)OB(c2ccc3cn[nH]c3c2)OC1(C)C] |

### Example 2A

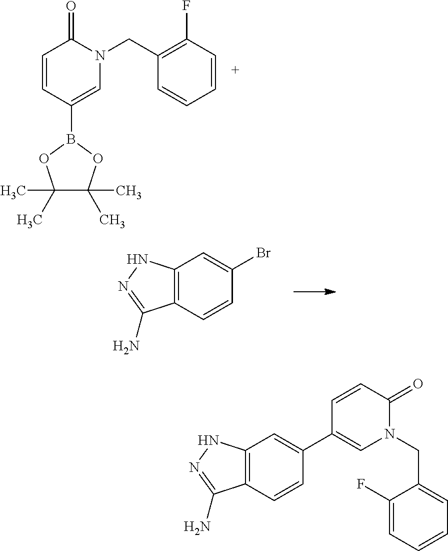 [SMILES: CC1(C)OB(c2ccc(=O)n(Cc3ccccc3F)c2)OC1(C)C ; Nc1n[nH]c2cc(Br)ccc12 ; Nc1n[nH]c2cc(-c3ccc(=O)n(Cc4ccccc4F)c3)ccc12]

### 5-(3-amino-1H-indazol-6-yl)-1-(2-fluorobenzyl)pyridin-2(1H)-one

A 1-[(2-fluorophenyl)methyl]-5-(tetramethyl-1,3,2-dioxaborolan-2-yl)-1,2-dihydropyridin-2-one (Method 2AD) (0.4 g, 1.2 mmol), 6-bromo-1H-indazol-3-amine (0.25 g, 1.2 mmol), caesium carbonate (0.9 g, 3 mmol) in mixture dioxane/water (2:1) were flushed with argon for 10 minutes and [1,1′-bis(diphenylphosphino)ferrocene]palladium(II) chloride, complex with dichloromethane (0.06 g) was added. The reaction mixture was heated at 125° C. under microwave irradiation until the reaction was completed. After cooled to ambient temperature reaction mixture was filtered through Celite, washed with ethyl acetate and solvents were evaporated under reduced pressure. Crude product was purified by flash chromatography (dichloromethane/methanol 95:5) to give 5-(3-amino-1H-indazol-6-yl)-1-(2-fluorobenzyl)pyridin-2(1H)-one (0.19 g); yield 48%. LC-MS (m/z) 335.1 (M+1). ¹H NMR (400 MHz, DMSO) δ 11.46 (s, 1H), 8.23 (d, J=2.5 Hz, 1H), 7.92 (dd, J=9.5, 2.7 Hz, 1H), 7.73 (d, J=8.4 Hz, 1H), 7.41-7.31 (m, 2H), 7.25 (d, J=10.7 Hz, 1H), 7.20-7.16 (m, 2H), 7.11 (dd, J=8.4, 1.3 Hz, 1H), 6.54 (d, J=9.5 Hz, 1H), 5.38 (s, 2H), 5.26 (s, 2H).

The following examples were prepared by the procedure of Example 2A, using the appropriate starting materials.

| Ex. | Product | ¹H NMR | m/z | Starting materials |
|---|---|---|---|---|
| 2B | 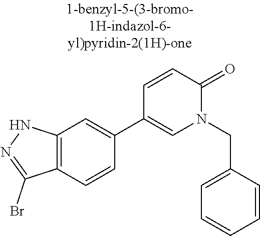 [SMILES: O=c1ccc(-c2ccc3c(Br)n[nH]c3c2)cn1Cc1ccccc1] | | 380.4 | 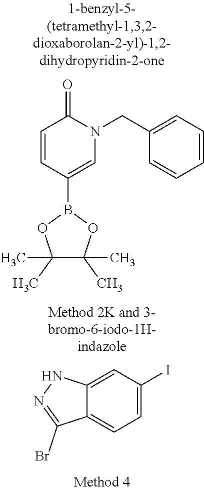 [SMILES: CC1(C)OB(c2ccc(=O)n(Cc3ccccc3)c2)OC1(C)C ; Brc1n[nH]c2cc(I)ccc12] |
| 2C | 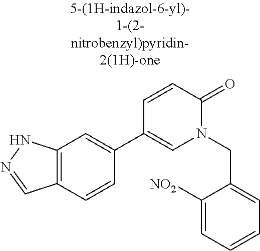 [SMILES: O=c1ccc(-c2ccc3cn[nH]c3c2)cn1Cc1ccccc1[N+](=O)[O-]] | | 347.3 | 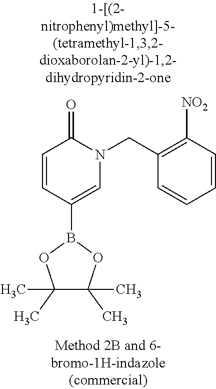 [SMILES: CC1(C)OB(c2ccc(=O)n(Cc3ccccc3[N+](=O)[O-])c2)OC1(C)C] |
| 2F | 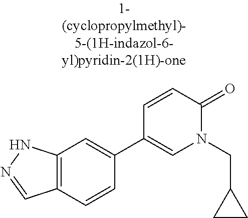 [SMILES: O=c1ccc(-c2ccc3cn[nH]c3c2)cn1CC1CC1] | (400 MHz, DMSO-d6) δ 13.11 (s, 1H), 8.20 (d, J = 2.7 Hz, 1H), 8.07 (s, 1H), 7.89 (dd, J = 9.4, 2.7 Hz, 1H), 7.81 (d, J = 8.4 Hz, 1H), 7.65 (s, 1H), 7.34 (dd, J = 8.5, 1.3 Hz, 1H), 6.52 (d, J = 9.4 Hz, 1H), 3.86 (d, J = 7.2 Hz, 2H), 1.34-1.28 | 266.1 | 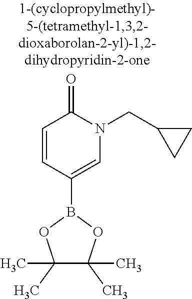 [SMILES: CC1(C)OB(c2ccc(=O)n(CC3CC3)c2)OC1(C)C] |
| | | (m, 1H), 0.53- | | |
| | | 0.42 (m, 4H). | | |
| 2G | 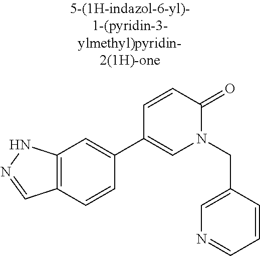 [SMILES: O=c1ccc(-c2ccc3cn[nH]c3c2)cn1Cc1cccnc1] | (300 MHz, DMSO) δ 13.09 (s, 1H), 8.65 (s, 1H), 8.48 (d, J = 3.9 Hz, 1H), 8.39 (d, J = 2.6 Hz, 1H), 8.05 (s, 1H), 7.91 (dd, J = 9.5, 2.6 Hz, 1H), 7.77 (t, J = 8.9 Hz, 2H), 7.64 (s, 1H), 7.40- 7.28 (m, 2H), 6.53 (d, J = 9.5 Hz, 1H), 5.21 (s, 2H) | 303.2 | 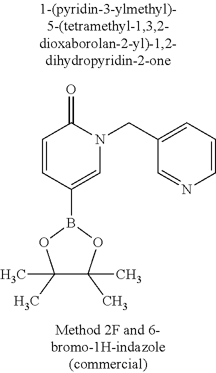 [SMILES: CC1(C)OB(c2ccc(=O)n(Cc3cccnc3)c2)OC1(C)C] |
| 2I | 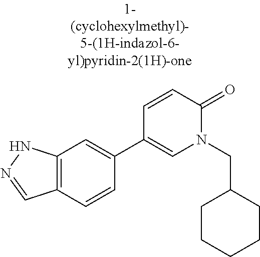 [SMILES: O=c1ccc(-c2ccc3cn[nH]c3c2)cn1CC1CCCCC1] | (400 MHz, DMSO-d6) δ 13.09 (s, 1H), 8.17-8.00 (m, 2H), 7.88 (dd, J = 9.4, 2.7 Hz, 1H), 7.80 (d, J = 8.4 Hz, 1H), 7.64 (s, 1H), 7.33 (d, J = 8.4 Hz, 1H), 6.50 (d, J = 9.4 Hz, 1H), 3.85 (d, J = 7.3 Hz, 2H), 1.90-1.77 (m, 1H), 1.71- 1.67 (m, 1H), 1.66- 1.54 (m, 3H), 1.25-1.15 (m, 4H), 1.09- 0.97 (m, 2H). | 308.1 | 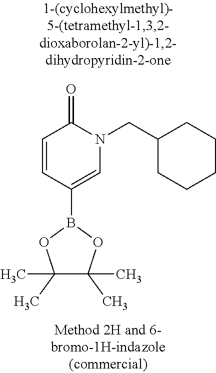 [SMILES: CC1(C)OB(c2ccc(=O)n(CC3CCCCC3)c2)OC1(C)C] |
| 2J | 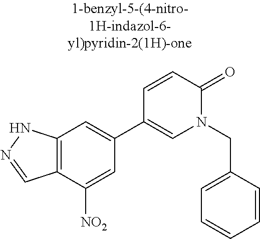 [SMILES: O=c1ccc(-c2cc([N+](=O)[O-])c3cn[nH]c3c2)cn1Cc1ccccc1] | | 347.1 | 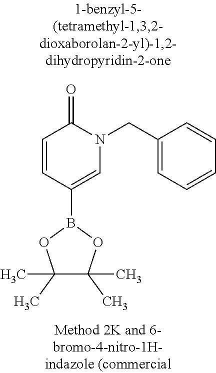 [SMILES: CC1(C)OB(c2ccc(=O)n(Cc3ccccc3)c2)OC1(C)C] |
| 2M | 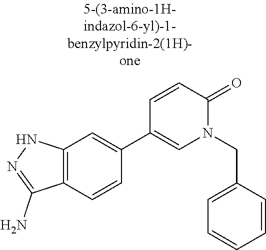 [SMILES: Nc1n[nH]c2cc(-c3ccc(=O)n(Cc4ccccc4)c3)ccc12] | (300 MHz, DMSO) δ 8.24 (d, J = 2.4 Hz, 1H), 7.85 (dd, J = 9.5, 2.7 Hz, 1H), 7.69 (dd, J = 8.4, 0.6 Hz, 1H), 7.40-7.21 (m, 5H), 7.08 (dd, J = 8.4, 1.5 Hz, 1H), 6.63-6.59 (m, 2H), 6.54- 6.48 (m, 1H), 5.18 (s, 2H) | 317.1 | 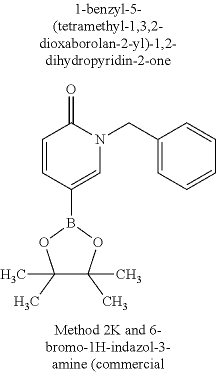 [SMILES: CC1(C)OB(c2ccc(=O)n(Cc3ccccc3)c2)OC1(C)C] |
| 2N | 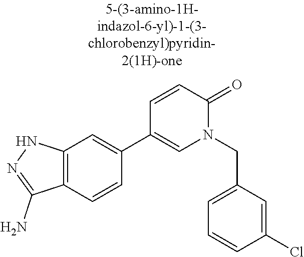 [SMILES: Nc1n[nH]c2cc(-c3ccc(=O)n(Cc4cccc(Cl)c4)c3)ccc12] | (400 MHz, DMSO) δ 11.46 (s, 1H), 8.33 (d, J = 2.6 Hz, 1H), 7.91 (dd, J = 9.5, 2.7 Hz, 1H), 7.73 (d, J = 8.4 Hz, 1H), 7.47 (s, 1H), 7.43- 7.32 (m, 4H), 7.13 (dd, J = 8.4, 1.3 Hz, 1H), 6.55 (d, J = 9.5 Hz, 1H), 5.38 (s, 2H), 5.20 (s, 2H) | 351.2 | 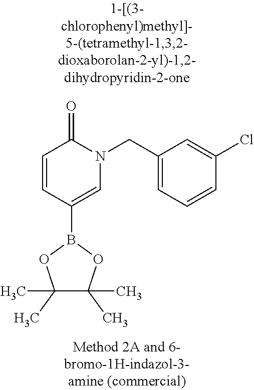 [SMILES: CC1(C)OB(c2ccc(=O)n(Cc3cccc(Cl)c3)c2)OC1(C)C] |
| 2O | 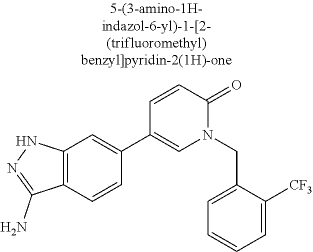 [SMILES: Nc1n[nH]c2cc(-c3ccc(=O)n(Cc4ccccc4C(F)(F)F)c3)ccc12] | (400 MHz, DMSO-d6) δ 11.44 (s, 1H), 8.25 (d, J = 2.6 Hz, 1H), 8.01 (dd, J = 9.5, 2.7 Hz, 1H), 7.81 (d, J = 7.6 Hz, 1H), 7.72 (d, J = 8.4 Hz, 1H), 7.64 (t, J = 7.7 Hz, 1H), 7.51 (t, J = 7.7 Hz, 1H), 7.38 (s, 1H), 7.14 (dd, J = 8.4, 1.3 Hz, 1H), 6.91 (d, J = 7.8 Hz, 1H), 6.61 (d, J = 9.5 Hz, 1H), 5.43 (s, 2H), 5.35 (s, 2H). | 385.1 | 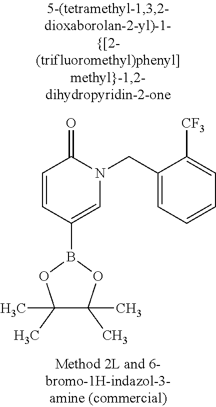 [SMILES: CC1(C)OB(c2ccc(=O)n(Cc3ccccc3C(F)(F)F)c2)OC1(C)C] |
| 2P | 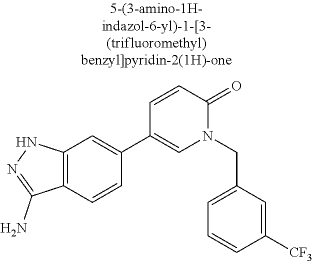 [SMILES: Nc1n[nH]c2cc(-c3ccc(=O)n(Cc4cccc(C(F)(F)F)c4)c3)ccc12] | (400 MHz, DMSO) δ 8.53 (d, J = 2.5 Hz, 1H), 8.03 (d, J = 8.6 Hz, 1H), 7.97 (dd, J = 9.5, 2.6 Hz, 1H), 7.79 (s, 1H), 7.71- 7.65 (m, 2H), 7.61 (d, J = 7.7 Hz, 1H), 7.58 (s, 1H), 7.43 (d, J = 9.9 Hz, 1H), 6.58 (d, J = 9.5 Hz, 1H), 5.29 (s, 2H) | 385.1 | 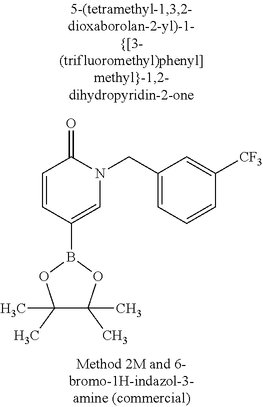 [SMILES: CC1(C)OB(c2ccc(=O)n(Cc3cccc(C(F)(F)F)c3)c2)OC1(C)C] |
| 2R | 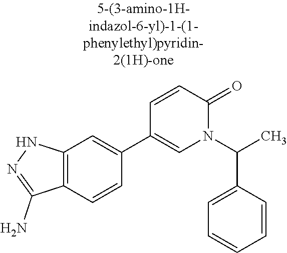 [SMILES: CC(c1ccccc1)n1cc(-c2ccc3c(N)n[nH]c3c2)ccc1=O] | (400 MHz, DMSO) δ 7.93 (d, J = 2.6 Hz, 1H), 7.88-7.83 (m, 2H), 7.40 (t, J = 6.9 Hz, 5H), 7.32- 7.28 (m, 1H), 7.25 (d, J = 8.9 Hz, 1H), 6.57 (d, J = 9.4 Hz, 1H), 6.25 (q, J = 7.1 Hz, 1H), 1.82 (d, J = 7.2 Hz, 3H) | 331.2 | 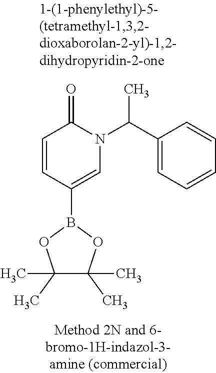 [SMILES: CC(c1ccccc1)n1cc(B2OC(C)(C)C(C)(C)O2)ccc1=O] |
| 2S | 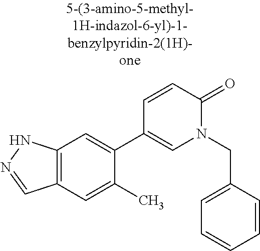 [SMILES: Cc1cc2cn[nH]c2cc1-c1ccc(=O)n(Cc2ccccc2)c1] | | 316.3 | 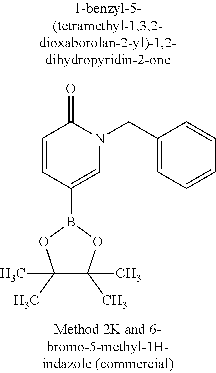 [SMILES: CC1(C)OB(c2ccc(=O)n(Cc3ccccc3)c2)OC1(C)C] |
| 2T | 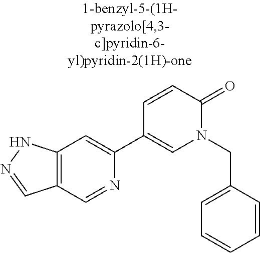 [SMILES: O=c1ccc(-c2cc3[nH]ncc3cn2)cn1Cc1ccccc1] | (300 MHz, DMSO) δ 13.49 (s, 1H), 9.07 (s, 1H), 8.68 (d, J = 2.5 Hz, 1H), 8.25 (dd, J = 9.6, 2.6 Hz, 2H), 7.83 (s, 1H), 7.57- 7.45 (m, 1H), 7.34 (d, J = 4.4 Hz, 3H), 7.31-7.24 (m, 1H), 6.55 (d, J = 9.5 Hz, 1H), 5.24 (s, 2H) | 303.1 | 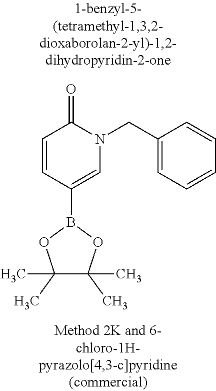 [SMILES: CC1(C)OB(c2ccc(=O)n(Cc3ccccc3)c2)OC1(C)C] |
| 2U | 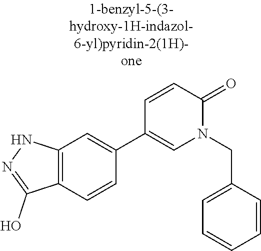 [SMILES: O=c1ccc(-c2ccc3c(O)n[nH]c3c2)cn1Cc1ccccc1] | | 318.1 | 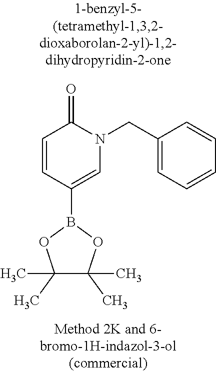 [SMILES: CC1(C)OB(c2ccc(=O)n(Cc3ccccc3)c2)OC1(C)C] |
| 2W | 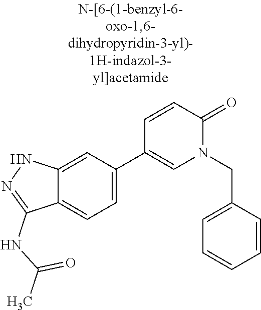 [SMILES: CC(=O)Nc1n[nH]c2cc(-c3ccc(=O)n(Cc4ccccc4)c3)ccc12] | | 359.4 | 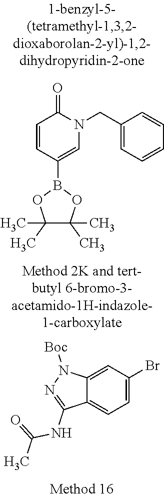 [SMILES: CC1(C)OB(c2ccc(=O)n(Cc3ccccc3)c2)OC1(C)C ; CC(=O)Nc1nn(C(=O)OC(C)(C)C)c2cc(Br)ccc12] |
| 2X | 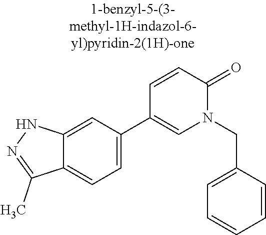 [SMILES: Cc1n[nH]c2cc(-c3ccc(=O)n(Cc4ccccc4)c3)ccc12] | (300 MHz, DMSO) δ 12.66 (s, 1H), 8.30 (d, J = 2.4 Hz, 1H), 7.89 (dd, J = 9.5, 2.7 Hz, 1H), 7.71 (d, J = 8.4 Hz, 1H), 7.53 (s, 1H), 7.41- 7.20 (m, 5H), 6.53 (d, J = 9.4 Hz, 1H), 5.19 (s, 2H), 1.12-0.89 (m, 4H) | 316.2 | 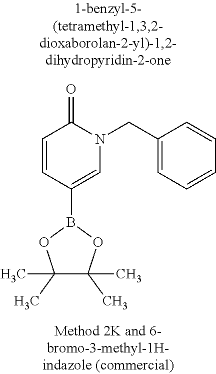 [SMILES: CC1(C)OB(c2ccc(=O)n(Cc3ccccc3)c2)OC1(C)C] |
| 2Y | 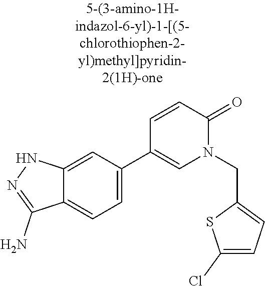 [SMILES: Nc1n[nH]c2cc(-c3ccc(=O)n(Cc4ccc(Cl)s4)c3)ccc12] | | 357.0 | 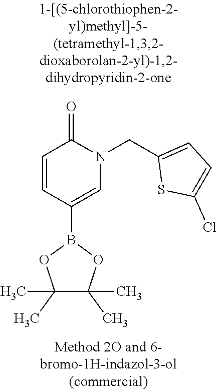 [SMILES: CC1(C)OB(c2ccc(=O)n(Cc3ccc(Cl)s3)c2)OC1(C)C] |
| 2Z | 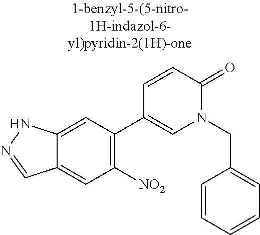 [SMILES: O=c1ccc(-c2cc3[nH]ncc3cc2[N+](=O)[O-])cn1Cc1ccccc1] | (300 MHz, DMSO) δ 13.70 (s, 8H), 8.65 (s, 5H), 8.38-8.32 (m, 5H), 8.06 (d, J = 2.3 Hz, 7H), 7.56 (s, 6H), 7.41 (dd, J = 9.4, 2.7 Hz, 7H), 7.36-7.23 (m, 26H), 6.45 (d, J = 9.6 Hz, 6H), 5.14 (s, 11H), 3.15 (s, 10H) | 347.2 | 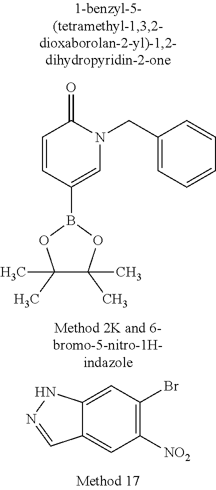 [SMILES: CC1(C)OB(c2ccc(=O)n(Cc3ccccc3)c2)OC1(C)C ; O=[N+]([O-])c1cc2cn[nH]c2cc1Br] |
| 2AA | 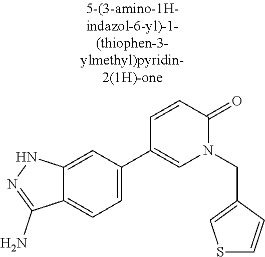 [SMILES: Nc1n[nH]c2cc(-c3ccc(=O)n(Cc4ccsc4)c3)ccc12] | (300 MHz, DMSO) δ 11.41 (s, 1H), 8.23 (d, J = 2.6 Hz, 1H), 7.83 (dd, J = 9.5, 2.7 Hz, 1H), 7.69 (d, J = 8.4 Hz, 1H), 7.48 (d, J = 4.4 Hz, 2H), 7.29 (s, 1H), 7.11 (ddd, J = 9.8, 6.4, 1.7 Hz, 2H), 6.50 (d, J = 9.4 Hz, 1H), 5.33 (s, 2H), 5.15 (s, 2H), 3.30 (s, 5H), 3.15 (d, J = 5.2 Hz, 2H) | 323.0 | 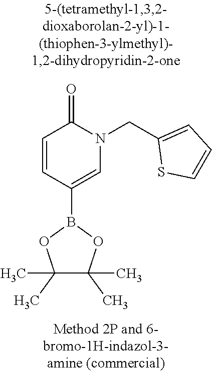 [SMILES: CC1(C)OB(c2ccc(=O)n(Cc3cccs3)c2)OC1(C)C] |
| 2AB | 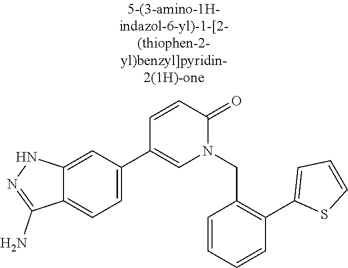 [SMILES: Nc1n[nH]c2cc(-c3ccc(=O)n(Cc4ccccc4-c4cccs4)c3)ccc12] | | 399.0 | 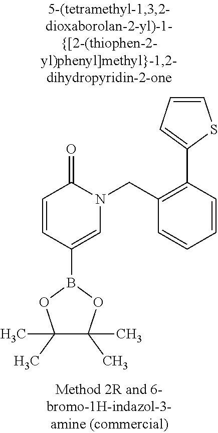 [SMILES: CC1(C)OB(c2ccc(=O)n(Cc3ccccc3-c3cccs3)c2)OC1(C)C] |
| 2AC | 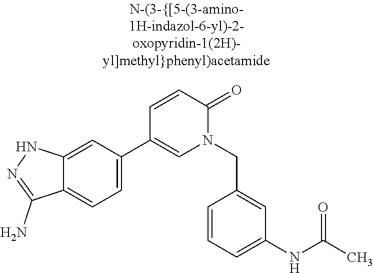 [SMILES: CC(=O)Nc1cccc(Cn2cc(-c3ccc4c(N)n[nH]c4c3)ccc2=O)c1] | (400 MHz, DMSO) δ 9.96 (s, 1H), 8.31 (d, J = 2.6 Hz, 1H), 7.93 (dd, J = 9.5, 2.7 Hz, 1H), 7.85 (d, J = 8.5 Hz, 1H), 7.56 (d, J = 8.4 Hz, 1H), 7.47 (d, J = 11.8 Hz, 2H), 7.27 (t, J = 8.0 Hz, 2H), 7.03 (d, J = 7.7 Hz, 1H), 6.56 (d, J = 9.5 Hz, 1H), 5.18 (s, 1H), 2.01 (s, 3H) | 374.1 | 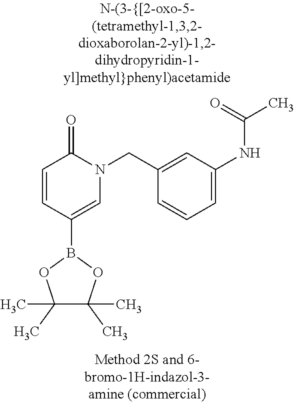 [SMILES: CC(=O)Nc1cccc(Cn2cc(B3OC(C)(C)C(C)(C)O3)ccc2=O)c1] |
| 2AD | 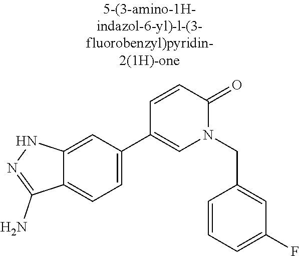 [SMILES: Nc1n[nH]c2cc(-c3ccc(=O)n(Cc4cccc(F)c4)c3)ccc12] | (400 MHz, DMSO) δ 11.45 (s, 1H), 8.31 (d, J = 2.6 Hz, 1H), 7.90 (dd, J = 9.5, 2.7 Hz, 1H), 7.73 (d, J = 8.4 Hz, 1H), 7.41 (td, J = 8.1, 6.3 Hz, 1H), 7.35 (s, 1H), 7.24- 7.19 (m, 2H), 7.12 (dd, J = 8.4, 1.4 Hz, 2H), 6.55 (d, J = 9.5 Hz, 1H), 5.37 (s, 2H), 5.21 (s, 2H) | 335.2 | 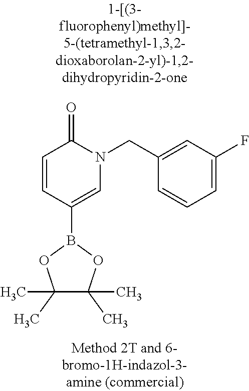 [SMILES: CC1(C)OB(c2ccc(=O)n(Cc3cccc(F)c3)c2)OC1(C)C] |
| 2AE | 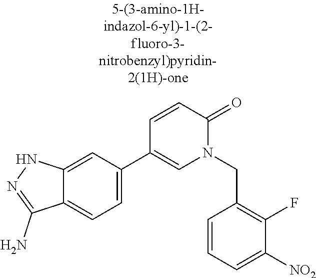 [SMILES: Nc1n[nH]c2cc(-c3ccc(=O)n(Cc4cccc([N+](=O)[O-])c4F)c3)ccc12] | (400 MHz, DMSO) δ 11.48 (s, 1H), 8.32 (d, J = 2.4 Hz, 1H), 8.09 (dd, J = 11.1, 4.2 Hz, 1H), 7.95 (dd, J = 9.5, 2.7 Hz, 1H), 7.74 (d, J = 8.4 Hz, 1H), 7.61 (t, J = 6.4 Hz, 1H), 7.42 (t, J = 8.0 Hz, 1H), 7.37 (s, 1H), 7.14 (dd, J = 8.4, 1.3 Hz, 1H), 6.55 (d, J = 9.5 Hz, 1H), 5.38 (s, 2H), 5.34 (s, 2H) | 380 | 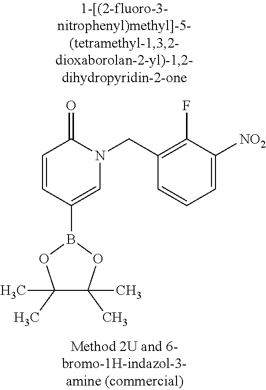 [SMILES: CC1(C)OB(c2ccc(=O)n(Cc3cccc([N+](=O)[O-])c3F)c2)OC1(C)C] |
| 2AF | 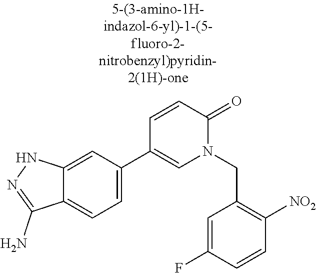 [SMILES: Nc1n[nH]c2cc(-c3ccc(=O)n(Cc4cc(F)ccc4[N+](=O)[O-])c3)ccc12] | (400 MHz, DMSO) δ 11.47 (s, 1H), 8.31-8.23 (m, 2H), 8.00 (dd, J = 9.5, 2.6 Hz, 1H), 7.73 (d, J = 8.4 Hz, 1H), 7.49-7.41 (m, 1H), 7.37 (s, 1H), 7.13 (d, J = 8.4 Hz, 1H), 6.82 (dd, J = 9.6, 2.7 Hz, 1H), 6.59 (d, J = 9.5 Hz, 1H), 5.52 (s, 2H), 5.38 (s, 2H) | 380.0 | 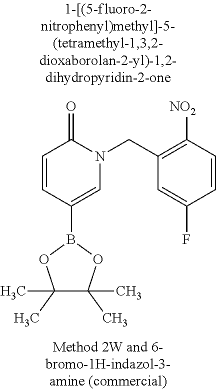 [SMILES: CC1(C)OB(c2ccc(=O)n(Cc3cc(F)ccc3[N+](=O)[O-])c2)OC1(C)C] |
| 2AG | 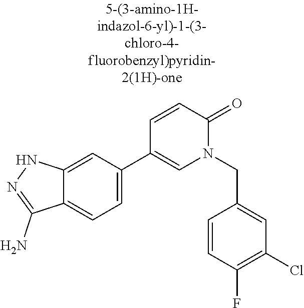 [SMILES: Nc1n[nH]c2cc(-c3ccc(=O)n(Cc4ccc(F)c(Cl)c4)c3)ccc12] | (400 MHz, DMSO) δ 8.41 (d, J = 2.6 Hz, 1H), 7.93 (dd, J = 9.5, 2.7 Hz, 1H), 7.85 (d, J = 8.3 Hz, 1H), 7.67 (dd, J = 7.2, 1.9 Hz, 1H), 7.48-7.40 (m, 3H), 7.28 (dd, J = 8.5, 1.4 Hz, 1H), 6.56 (d, J = 9.4 Hz, 1H), 5.17 (s, 2H) | 369.1 | 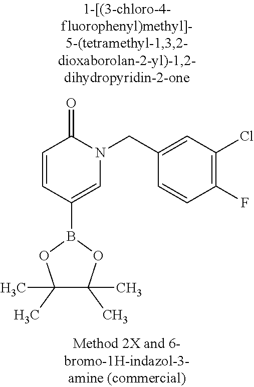 [SMILES: CC1(C)OB(c2ccc(=O)n(Cc3ccc(F)c(Cl)c3)c2)OC1(C)C] |
| 2AH | 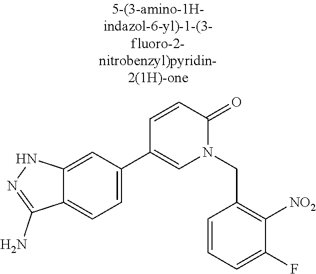 [SMILES: Nc1n[nH]c2cc(-c3ccc(=O)n(Cc4cccc(F)c4[N+](=O)[O-])c3)ccc12] | (400 MHz, DMSO) δ 8.39 (d, J = 2.6 Hz, 1H), 8.00 (dd, J = 9.5, 2.7 Hz, 1H), 7.86 (d, J = 8.5 Hz, 1H), 7.69 (td, J = 8.2, 5.6 Hz, 1H), 7.60- 7.53 (m, 1H), 7.48 (s, 1H), 7.29 (dd, J = 8.6, 1.3 Hz, 1H), 7.09 (d, J = 7.9 Hz, 1H), 6.56 (d, J = 9.5 Hz, 1H), 5.33 (s, 1H) | 380.1 | 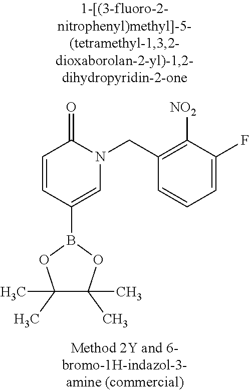 [SMILES: CC1(C)OB(c2ccc(=O)n(Cc3cccc(F)c3[N+](=O)[O-])c2)OC1(C)C] |
| 2AI | 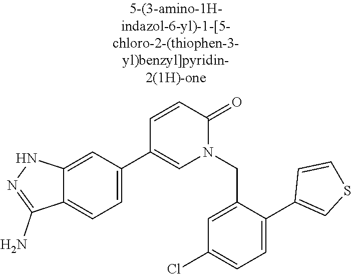 [SMILES: Nc1n[nH]c2cc(-c3ccc(=O)n(Cc4cc(Cl)ccc4-c4ccsc4)c3)ccc12] | (400 MHz, DMSO) δ 11.46 (s, 1H), 7.95 (dt, J = 9.3, 2.5 Hz, 2H), 7.73 (ddd, J = 5.0, 4.2, 2.5 Hz, 3H), 7.42- 7.39 (m, 2H), 7.39-7.35 (m, 1H), 7.31 (s, 1H), 7.07 (dd, J = 8.4, 1.4 Hz, 1H), 6.92 (d, J = 1.8 Hz, 1H), 6.54 (d, J = 9.3 Hz, 1H), 5.38 (s, 2H), 5.25 (s, 2H) | 433.0 | 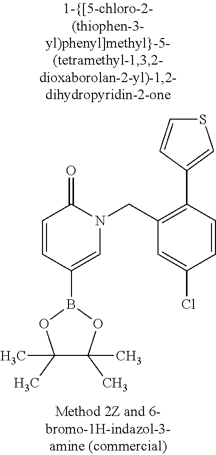 [SMILES: CC1(C)OB(c2ccc(=O)n(Cc3cc(Cl)ccc3-c3ccsc3)c2)OC1(C)C] |
| 2AJ | 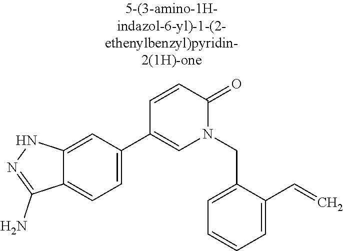 [SMILES: C=Cc1ccccc1Cn1cc(-c2ccc3c(N)n[nH]c3c2)ccc1=O] | (400 MHz, DMSO) δ 11.44 (s, 1H), 8.09 (d, J = 2.6 Hz, 1H), 7.92 (dd, J = 9.5, 2.7 Hz, 1H), 7.71 (d, J = 8.4 Hz, 1H), 7.62- 7.58 (m, 1H), 7.34-7.28 (m, 3H), 7.19 (dd, J = 17.3, 11.0 Hz, 1H), 7.07 (dd, J = 8.4, 1.4 Hz, 1H), 7.01- 6.97 (m, 1H), 6.57 (d, J = 9.5 Hz, 1H), 5.81-5.75 (m, 1H), 5.41 (dd, J = 11.0, 1.3 Hz, 1H), | 343.2 | 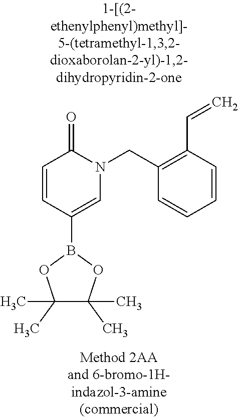 [SMILES: C=Cc1ccccc1Cn1cc(B2OC(C)(C)C(C)(C)O2)ccc1=O] |
| | | 5.37 (s, 2H), | | |
| | | 5.31 (s, 2H) | | |
| 2AK | 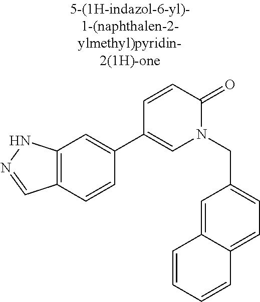 [SMILES: O=c1ccc(-c2ccc3cn[nH]c3c2)cn1Cc1ccc2ccccc2c1] | | 352.4 | 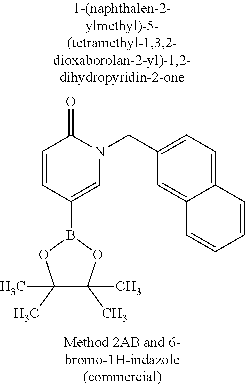 [SMILES: CC1(C)OB(c2ccc(=O)n(Cc3ccc4ccccc4c3)c2)OC1(C)C] |
| 2AL | 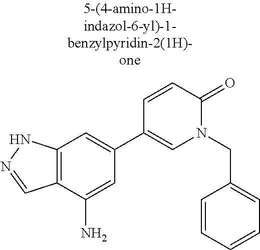 [SMILES: Nc1cc(-c2ccc(=O)n(Cc3ccccc3)c2)cc2[nH]ncc12] | (400 MHz, DMSO) δ 12.69 (s, 1H), 8.11 (d, J = 2.7 Hz, 1H), 8.07 (s, 1H), 7.76 (dd, J = 9.4, 2.7 Hz, 1H), 7.36 (d, J = 4.4 Hz, 4H), 7.29 (dd, J = 8.9, 4.5 Hz, 1H), 6.71 (s, 1H), 6.53 (d, J = 9.4 Hz, 1H), 6.26 (s, 1H), 5.83 (s, 2H), 5.21 (s, 2H) | 317.1 | 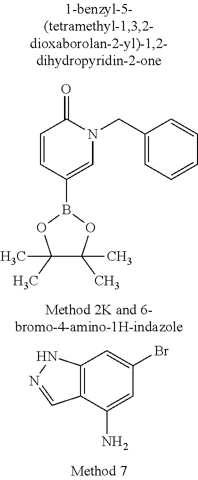 [SMILES: CC1(C)OB(c2ccc(=O)n(Cc3ccccc3)c2)OC1(C)C ; Nc1cc(Br)cc2[nH]ncc12] |
| 2AU | 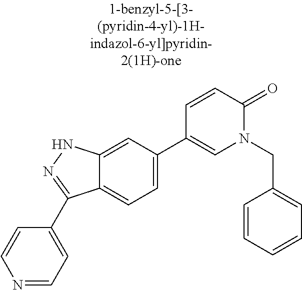 [SMILES: O=c1ccc(-c2ccc3c(-c4ccncc4)n[nH]c3c2)cn1Cc1ccccc1] | | 379.4 | 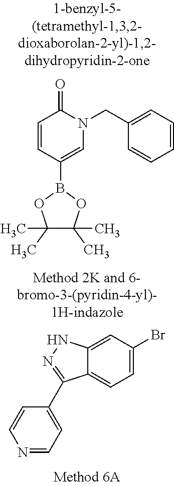 [SMILES: CC1(C)OB(c2ccc(=O)n(Cc3ccccc3)c2)OC1(C)C ; Brc1ccc2c(-c3ccncc3)n[nH]c2c1] |
| 2AW | 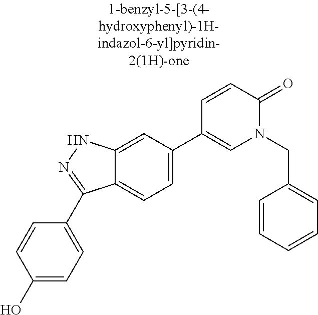 [SMILES: O=c1ccc(-c2ccc3c(-c4ccc(O)cc4)n[nH]c3c2)cn1Cc1ccccc1] | | 394.5 | 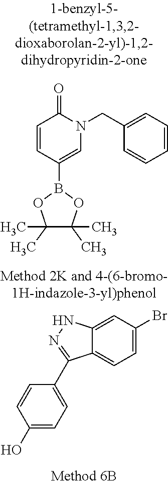 [SMILES: CC1(C)OB(c2ccc(=O)n(Cc3ccccc3)c2)OC1(C)C ; Oc1ccc(-c2n[nH]c3cc(Br)ccc23)cc1] |
| 2AX | 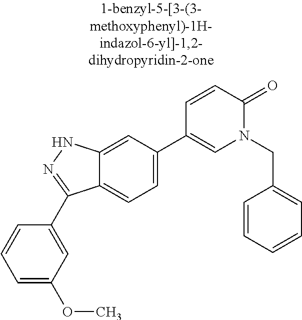 [SMILES: COc1cccc(-c2n[nH]c3cc(-c4ccc(=O)n(Cc5ccccc5)c4)ccc23)c1] | | 408.4 | 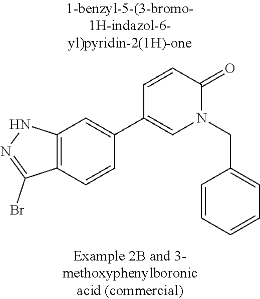 [SMILES: O=c1ccc(-c2ccc3c(Br)n[nH]c3c2)cn1Cc1ccccc1] |
| 2AY | 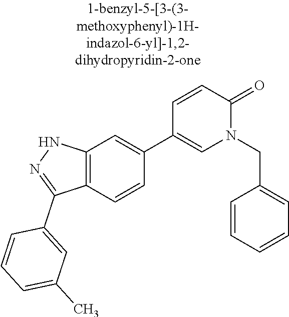 [SMILES: Cc1cccc(-c2n[nH]c3cc(-c4ccc(=O)n(Cc5ccccc5)c4)ccc23)c1] | | 392.4 | 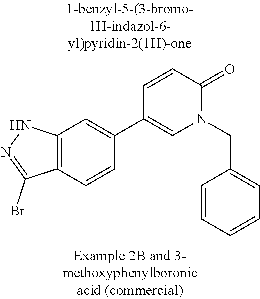 [SMILES: O=c1ccc(-c2ccc3c(Br)n[nH]c3c2)cn1Cc1ccccc1] |
| 2AZ | 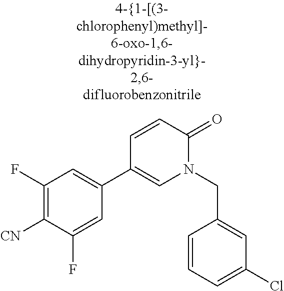 | | | 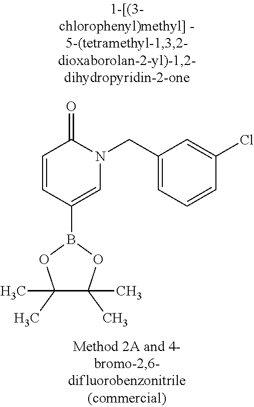 [SMILES: CC1(C)OB(c2ccc(=O)n(Cc3cccc(Cl)c3)c2)OC1(C)C] |
| 2BA | 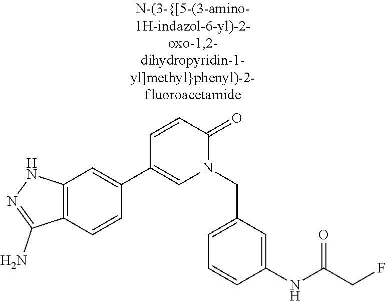 [SMILES: Nc1n[nH]c2cc(-c3ccc(=O)n(Cc4cccc(NC(=O)CF)c4)c3)ccc12] | 400 MHz, DMSO δ 11.44 (s, 1H), 10.12 (s, 1H), 8.32 (s, 1H), 8.24 (d, J = 2.6 Hz, 1H), 7.90 (dd, J = 9.5, 2.7 Hz, 1H), 7.72 (d, J = 8.4 Hz, 1H), 7.62 (d, J = 8.1 Hz, 1H), 7.56 (s, 1H), 7.32 (dd, J = 14.4, 6.6 Hz, 2H), 7.12 (dd, J = 11.1, 4.2 Hz, 2H), 6.54 (d, J = 9.4 Hz, 1H), 5.36 (s, 2H), 5.20 (s, | | 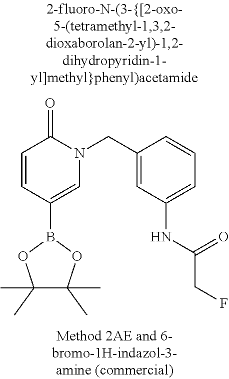 [SMILES: CC1(C)OB(c2ccc(=O)n(Cc3cccc(NC(=O)CF)c3)c2)OC1(C)C] |
| | | 2H), 5.01 (s, | | |
| | | 1H), 4.89 (s, | | |
| | | 1H). | | |
| 2BB | 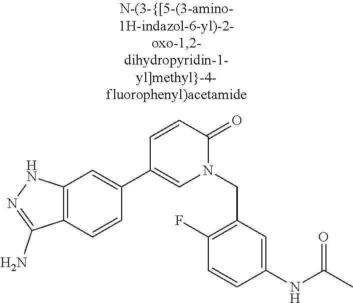 [SMILES: CC(=O)Nc1ccc(F)c(Cn2cc(-c3ccc4c(N)n[nH]c4c3)ccc2=O)c1] | 400 MHz, DMSO δ 11.45 (s, 1H), 9.96 (s, 1H), 8.22 (s, 1H), 7.94 (dd, J = 9.5, 2.5 Hz, 1H), 7.73 (d, J = 8.3 Hz, 1H), 7.69- 7.61 (m, 1H), 7.36 (s, 1H), 7.24 (d, J = 4.3 Hz, 1H), 7.15 (dd, J = 19.6, 9.2 Hz, 2H), 6.55 (d, J = 9.5 Hz, 1H), 5.36 (s, 2H), 5.22 (s, 2H), 1.97 (s, 3H). | | 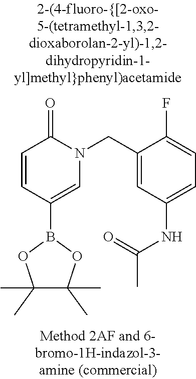 [SMILES: CC(=O)Nc1ccc(F)c(Cn2cc(B3OC(C)(C)C(C)(C)O3)ccc2=O)c1] |
| 2BC | 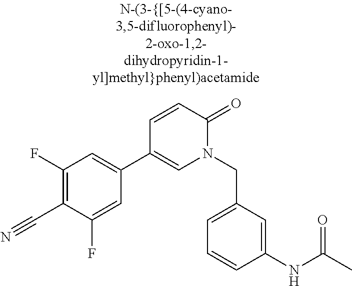 [SMILES: CC(=O)Nc1cccc(Cn2cc(-c3cc(F)c(C#N)c(F)c3)ccc2=O)c1] | | | 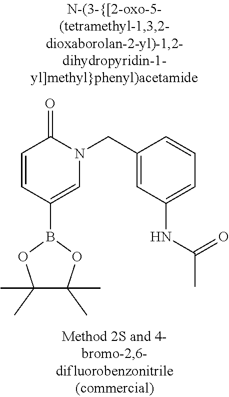 [SMILES: CC(=O)Nc1cccc(Cn2cc(B3OC(C)(C)C(C)(C)O3)ccc2=O)c1] |
| 2BD | 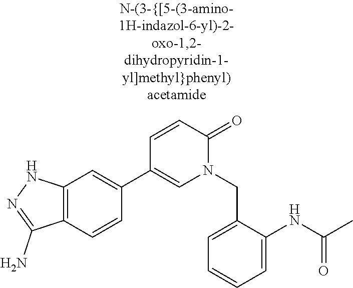 [SMILES: CC(=O)Nc1ccccc1Cn1cc(-c2ccc3c(N)n[nH]c3c2)ccc1=O] | 400 MHz, DMSO δ 11.47 (s, 1H), 10.37 (s, 1H), 8.33 (d, J = 2.5 Hz, 1H), 7.96 (dd, J = 9.4, 2.6 Hz, 1H), 7.73 (d, J = 8.3 Hz, 2H), 7.46 (d, J = 7.6 Hz, 1H), 7.36 (s, 1H), 7.33- 7.28 (m, 1H), 7.13 (dd, J = 11.3, 4.1 Hz, 2H), 6.63 (d, J = 9.4 Hz, 1H), 5.37 (s, 2H), 5.22 (s, 2H), 2.13 (s, 3H). | | 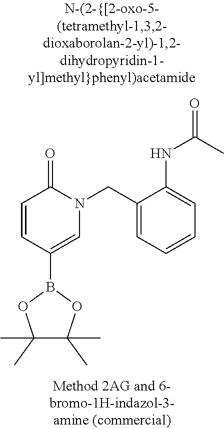 [SMILES: CC(=O)Nc1ccccc1Cn1cc(B2OC(C)(C)C(C)(C)O2)ccc1=O] |
| 2BE | 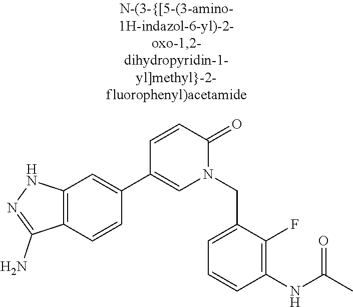 [SMILES: CC(=O)Nc1cccc(Cn2cc(-c3ccc4c(N)n[nH]c4c3)ccc2=O)c1F] | (400 MHz, DMSO) δ 11.45 (s, 1H), 9.75 (s, 1H), 8.23 (d, J = 2.5 Hz, 1H), 7.91 (dd, J = 9.5, 2.7 Hz, 1H), 7.82 (t, J = 7.6 Hz, 1H), 7.73 (d, J = 8.4 Hz, 1H), 7.35 (s, 1H), 7.11 (dd, J = 12.0, 4.8 Hz, 2H), 6.94 (t, J = 6.7 Hz, 1H), 6.53 (d, J = 9.5 Hz, 1H), 5.36 (s, 2H), 5.26 (s, 2H), 2.08 (s, 3H). | | 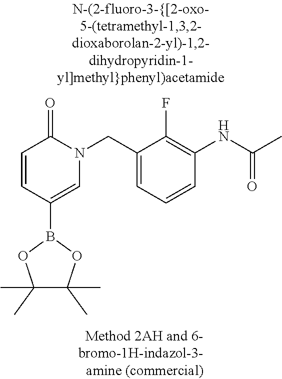 [SMILES: CC(=O)Nc1cccc(Cn2cc(B3OC(C)(C)C(C)(C)O3)ccc2=O)c1F] |
| 2BF | 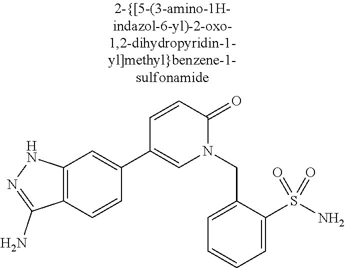 [SMILES: Nc1n[nH]c2cc(-c3ccc(=O)n(Cc4ccccc4S(N)(=O)=O)c3)ccc12] | (400 MHz, DMSO) 5 11.44 (s, 1H), 8.21 (d, J = 2.6 Hz, 1H), 7.98 (ddd, J = 9.1, 8.7, 2.0 Hz, 2H), 7.74- 7.65 (m, 3H), 7.56 (dt, J = 7.6, 3.8 Hz, 1H), 7.49 (t, J = 7.1 Hz, 1H), 7.36 (s, 1H), 7.12 (dd, J = 8.4, 1.3 Hz, 1H), 7.00 (d, J = 7.6 Hz, 1H), 6.63 (d, J = 9.5 Hz, 1H), 5.64 (s, 2H), 5.35 (s, 2H). | | 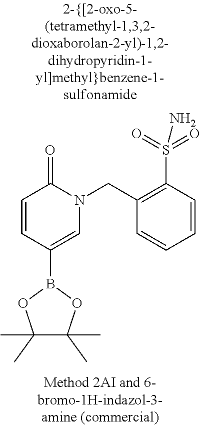 [SMILES: CC1(C)OB(c2ccc(=O)n(Cc3ccccc3S(N)(=O)=O)c2)OC1(C)C] |

### Example 3A

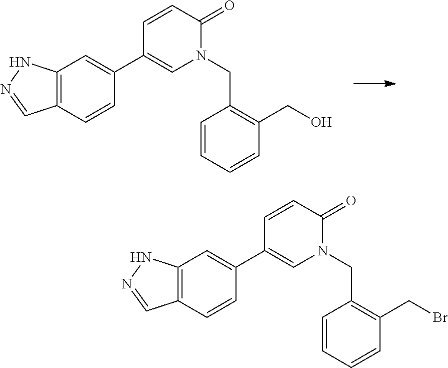 [SMILES: O=c1ccc(-c2ccc3cn[nH]c3c2)cn1Cc1ccccc1CO ; O=c1ccc(-c2ccc3cn[nH]c3c2)cn1Cc1ccccc1CBr]

### 1-[2-(bromomethyl)benzyl]-5-(1H-indazol-6-yl)pyridin-2(1H)-one

To a solution of 1-[2-(hydroxymethyl)benzyl]-5-(1H-indazol-6-yl)pyridin-2(1H)-one (Example 1AW) (0.06 g, 0.2 mmol) in dry acetonitrile (2 mL), cooled to 0° C., phosphorus tribromide (0.01 mL, 0.1 mmol) was dropped. The reaction mixture was heated at 110° C. for 3 h and then stirred at room temperature overnight. Then to the cooled to 0° C. reaction mixture water was added. The precipitated product was collected by filtration and purified by flash chromatography (dichloromethane/methanol 95:5) to give 1-[2-(bromomethyl)benzyl]-5-(1H-indazol-6-yl)pyridin-2(1H)-one (0.06 g) as a white solid; yield 75%. LC-MS (m/z) 394.1 (M+1).

The following examples were prepared by the procedure of Example 3A, using the appropriate starting materials.

| Ex. | Product | ¹H NMR | m/z | Starting materials |
|---|---|---|---|---|
| 3B | 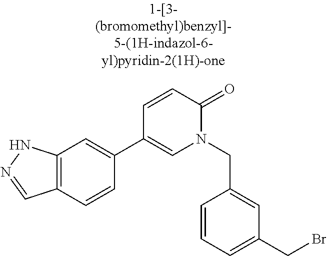 [SMILES: O=c1ccc(-c2ccc3cn[nH]c3c2)cn1Cc1cccc(CBr)c1] | (400 MHz, DMSO) δ 8.36 (d, J = 2.6 Hz, 1H), 8.08 (d, J = 0.9 Hz, 1H), 7.94 (ddd, J = 8.5, 5.7, 2.7 Hz, 1H), 7.81 (dd, J = 8.4, 0.6 Hz, 1H), 7.66 (d, J = 3.7 Hz, 1H), 7.47 (s, 1H), 7.39-7.30 (m, 6H), 6.57 (dd, J = 9.4, 2.3 Hz, 1H), 5.22 (s, J = 8.3 Hz, 2H) | 394.3 | 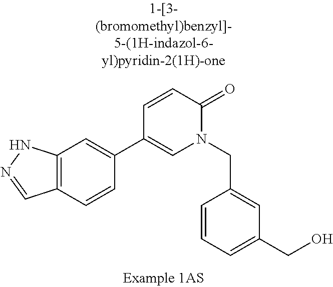 [SMILES: O=c1ccc(-c2ccc3cn[nH]c3c2)cn1Cc1cccc(CO)c1] |

### Example 4A

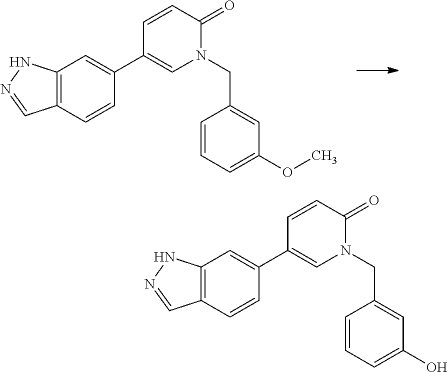

### 1-(3-hydroxybenzyl)-5-(1H-indazol-6-yl)pyridin-2(1H)-one

To a solution of 5-(1H-indazol-6-yl)-1-(3-methoxybenzyl)pyridin-2(1H)-one (Example 1I) (0.07, 0.2 mmol) in dry dichloromethane (1.5 mL), cooled to −78° C., boron tribromide (0.1 mL, 1 mmol) was dropped. Then the reaction mixture was slowly warmed up to room temperature and stirred overnight. Next methanol (5 mL) was added and the reaction mixture was evaporated under reduced pressure to dryness. The crude product was purified by column chromatography (silica gel, dichloromethane/methanol 98:2) to give 1-(3-hydroxybenzyl)-5-(1H-indazol-6-yl)pyridin-2(1H)-one (0.01 g); yield 15%. LC-MS (m/z) 318.1 (M+1); ¹H NMR (400 MHz, DMSO) δ 13.11 (s, 1H), 8.27 (d, J=2.6 Hz, 1H), 8.07 (s, 1H), 7.93 (dd, J=9.5, 2.7 Hz, 1H), 7.80 (d, J=8.4 Hz, 1H), 7.64 (s, 1H), 7.33 (dd, J=8.5, 1.4 Hz, 1H), 7.13 (t, J=7.8 Hz, 1H), 6.80 (d, J=7.6 Hz, 1H), 6.75 (s, 1H), 6.66 (dd, J=7.8, 2.1 Hz, 1H), 6.56 (d, J=9.4 Hz, 1H), 5.13 (s, 2H).

The following examples were prepared by the procedure of Example 4A, using the appropriate starting materials.

| Ex. | Product | ¹H NMR | m/z | Starting materials |
|---|---|---|---|---|
| 4B | 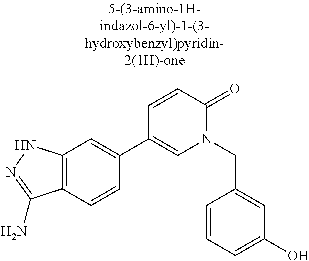 [SMILES: Nc1n[nH]c2cc(-c3ccc(=O)n(Cc4cccc(O)c4)c3)ccc12] | (300 MHz, DMSO) δ 9.42 (bs, 1H), 8.28 (d, J = 2.6 Hz, 1H), 7.90 (dd, J = 9.5, 2.7 Hz, 1H), 7.84 (d, J = 8.5 Hz, 1H), 7.44 (s 1H), 7.27 (dd, J = 8.5, 1.4 Hz, 1H), 7.14-7.07 (m, 1H), 6.76 (d, J = 7.7 Hz, 1H), 6.73- 6.69 (m, 1H), 6.64 (dd, J = 8.1, 1.6 Hz, 1H), 6.54 (d, J = 9.5 Hz, 1H), 5.10 (s, 2H) | 331.1 | 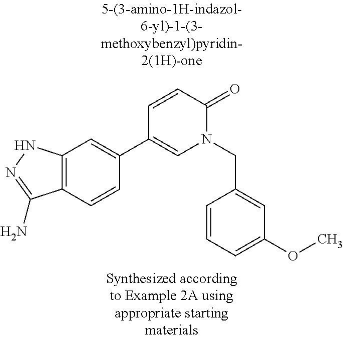 [SMILES: COc1cccc(Cn2cc(-c3ccc4c(N)n[nH]c4c3)ccc2=O)c1] |

### Example 5

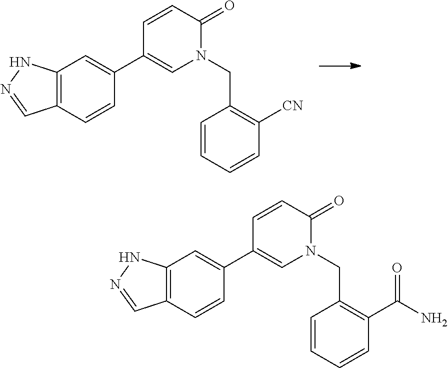 [SMILES: N#Cc1ccccc1Cn1cc(-c2ccc3cn[nH]c3c2)ccc1=O ; NC(=O)c1ccccc1Cn1cc(-c2ccc3cn[nH]c3c2)ccc1=O]

### 2-{[5-(1H-indazol-6-yl)-2-oxopyridin-1(2H)-yl]methyl}benzamide

A 2-{[5-(1H-indazol-6-yl)-2-oxopyridin-1(2H)-yl]methyl}benzonitrile (Example 1C) (0.06 g, 0.2 mmol) was dissolved in a mixture of methanol/water (5:1) then potassium carbonate (0.15 g, 1.1 mmol) was added. The reaction mixture was cooled to 0° C. and hydrogen peroxide 30% (0.14 mL, 4.2 mmol) was dropped. After warmed up to the room temperature stirring was continuous overnight. Then water and ethyl acetate were added to the mixture. Organic phase was separated, dried over anhydrous sodium sulfate, filtered and solvent was removed under reduced pressure. The product was purified by column chromatography (silica gel; dichloromethane/methanol 9:1) to give 2-{[5-(1H-indazol-6-yl)-2-oxopyridin-1(2H)-yl]methyl}benzamide (0.3 g); yield 46%. LC-MS (m/z) 345.2 (M+1); ¹H NMR (300 MHz, DMSO) δ 13.09 (s, 1H), 8.32 (d, J=2.5 Hz, 1H), 8.05 (dd, J=8.7, 3.9 Hz, 2H), 7.94 (dd, J=9.5, 2.7 Hz, 1H), 7.77 (dd, J=8.5, 0.7 Hz, 1H), 7.63 (s, 1H), 7.60-7.47 (m, 2H), 7.44-7.26 (m, 3H), 7.13-7.01 (m, 1H), 6.55 (d, J=9.5 Hz, 1H), 5.38 (s, 2H), 3.17-3.12 (m, 2H).

### Example 6A

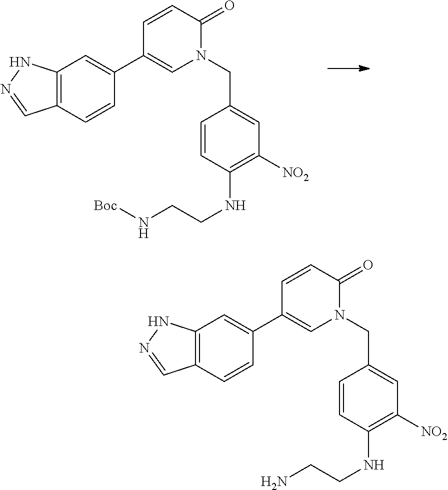 [SMILES: CC(C)(C)OC(=O)NCCNc1ccc(Cn2cc(-c3ccc4cn[nH]c4c3)ccc2=O)cc1[N+](=O)[O-] ; NCCNc1ccc(Cn2cc(-c3ccc4cn[nH]c4c3)ccc2=O)cc1[N+](=O)[O-]]

### 1-{4-[(2-aminoethyl)amino]-3-nitrobenzyl}-5-(1H-indazol-6-yl)pyridin-2(1H)-one hydrochloride

A tert-butyl N-{2-[(4-{[5-(1H-indazol-6-yl)-2-oxo-1,2-dihydropyridin-1-yl]methyl}-2-nitrophenyl)amino]ethyl}carbamate (Example 1BK) (0.07 g, 0.14 mmol) was dissolved in methanol (0.5 mL) and 4M HCl in dioxane (1 mL) was added. The reaction mixture was stirred at room temperature overnight. The precipitated product was collected by filtration, washed with diethyl ether and air dried to give of 1-{4-[(2-aminoethyl)amino]-3-nitrobenzyl}-5-(1H-indazol-6-yl)pyridin-2(1H)-one hydrochloride (0.05 g); yield 77%; LC-MS (m/z) 405.1 (M+1). ¹H NMR (400 MHz, DMSO) δ 8.46 (d, J=2.6 Hz, 1H), 8.27 (d, J=2.0 Hz, 1H), 8.21 (s, 1H), 8.07 (d, J=6.7 Hz, 1H), 8.03 (s, 2H), 7.91 (dd, J=9.5, 2.7 Hz, 1H), 7.82 (d, J=8.5 Hz, 1H), 7.71 (dd, J=8.9, 2.0 Hz, 1H), 7.66 (s, 1H), 7.34 (dd, J=8.5, 1.3 Hz, 1H), 7.19 (d, J=9.0 Hz, 1H), 6.55 (d, J=9.4 Hz, 1H), 5.14 (s, 2H), 3.67 (s, 2H), 2.98 (dd, J=11.8, 6.0 Hz, 2H).

The following examples were prepared by the procedure of Example 6A, using the appropriate starting materials.

| Ex. | Product | ¹H NMR | m/z | Starting material |
|---|---|---|---|---|
| 6B | 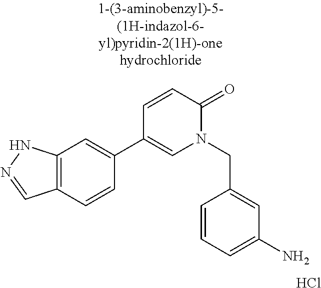 [SMILES: Nc1cccc(Cn2cc(-c3ccc4cn[nH]c4c3)ccc2=O)c1 ; Cl] | (400 MHz, DMSO) δ 13.12 (s, 1H), 8.20 (s, 1H), 8.07 (s, 1H), 7.86 (dd, J = 45.3, 8.7 Hz, 2H), 7.64 (s, 1H), 7.32 (d, J = 8.2 Hz, 1H), 6.98 (t, J = 7.4 Hz, 1H), 6.64- 6.41 (m, 4H), 5.11 (s, 2H), 5.07 (s, 2H) | 317.2 | 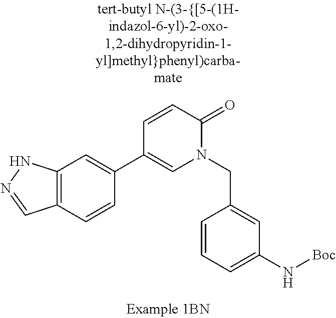 [SMILES: CC(C)(C)OC(=O)Nc1cccc(Cn2cc(-c3ccc4cn[nH]c4c3)ccc2=O)c1] |
| 6C | 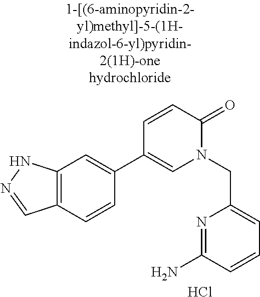 [SMILES: Nc1cccc(Cn2cc(-c3ccc4cn[nH]c4c3)ccc2=O)n1 ; Cl] | (300 MHz, DMSO) δ 8.43 (d, J = 2.5 Hz, 1H), 8.06 (s, 1H), 8.01 (dd, J = 9.5, 2.6 Hz, 1H), 7.83 (dd, J = 17.1, 6.8 Hz, 2H), 7.69 (s, 1H), 7.36 (dd, J = 8.5, 1.4 Hz, 1H), 6.90 (d, J = 8.8 Hz, 1H), 6.60- 6.54 (m, 2H), 5.24 (s, 2H) | 318.1 | 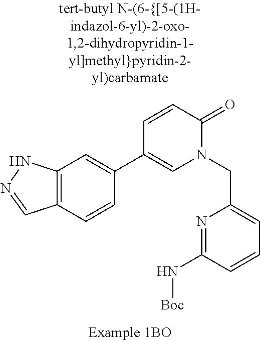 [SMILES: CC(C)(C)OC(=O)Nc1cccc(Cn2cc(-c3ccc4cn[nH]c4c3)ccc2=O)n1] |
| 6D | 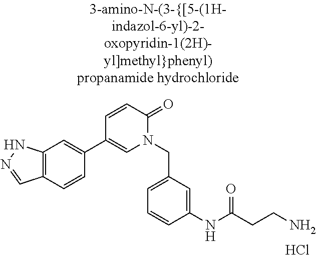 [SMILES: NCCC(=O)Nc1cccc(Cn2cc(-c3ccc4cn[nH]c4c3)ccc2=O)c1 ; Cl] | | 388.0 | 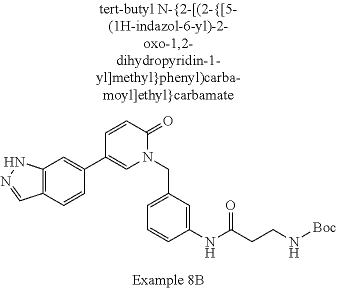 [SMILES: CC(C)(C)OC(=O)NCCC(=O)Nc1cccc(Cn2cc(-c3ccc4cn[nH]c4c3)ccc2=O)c1] |
| 6E | 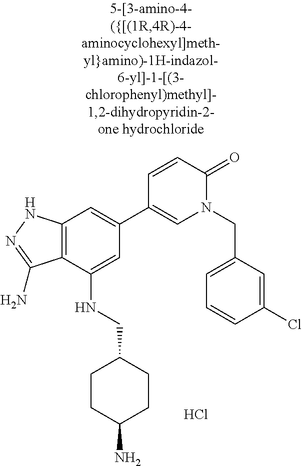 [SMILES: Nc1n[nH]c2cc(-c3ccc(=O)n(Cc4cccc(Cl)c4)c3)cc(NC[C@H]3CC[C@H](N)CC3)c12 ; Cl] | (400 MHz, DMSO) δ 11.33 (s, 1H), 8.43 (s, 1H), 8.18 (d, J = 2.5 Hz, 1H), 7.81 (dd, J = 9.4, 2.6 Hz, 1H), 7.45 (s, 1H), 7.37 (ddd, J = 13.2, 9.7, 6.8 Hz, 3H), 6.56- 6.49 (m, 2H), 5.95 (s, 1H), 5.56 (t, J = 5.6 Hz, 1H), 5.20 (s, 2H), 5.03 (s, 2H), 3.05 (t, J = 5.9 Hz, 2H), 2.83 (s, 1H), 1.92 (s, 4H), 1.63 (s, 1H), 1.23 (d, J = 13.2 Hz, 3H), 1.07 (t, J = 11.8 Hz, 2H) | 477.2 | 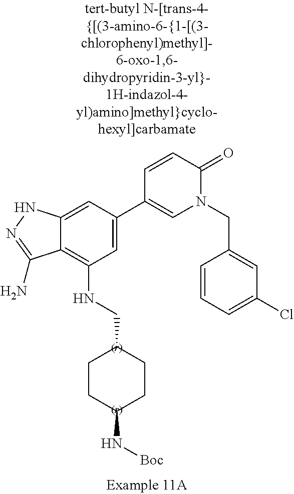 [SMILES: CC(C)(C)OC(=O)N[C@H]1CC[C@H](CNc2cc(-c3ccc(=O)n(Cc4cccc(Cl)c4)c3)cc3[nH]nc(N)c23)CC1] |
| 6F | 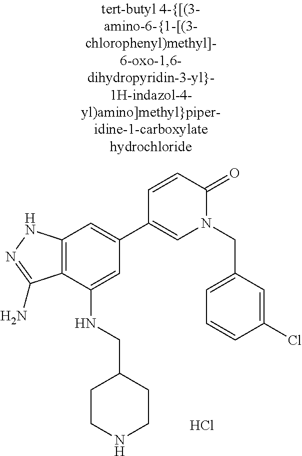 [SMILES: Nc1n[nH]c2cc(-c3ccc(=O)n(Cc4cccc(Cl)c4)c3)cc(NCC3CCNCC3)c12 ; Cl] | (400 MHz, DMSO) δ 11.34 (s, 1H), 8.40 (s, 1H), 8.19 (d, J = 2.5 Hz, 1H), 7.82 (dd, J = 9.5, 2.7 Hz, 1H), 7.45 (s, 1H), 7.43- 7.30 (m, 3H), 6.52 (d, J = 9.3 H, 2H), 5.98 (s, 1H), 5.63 (t, J = 5.5 Hz, 1H), 5.20 (s, 2H), 5.05 (s, 2H), 2.77-2.62 (m, J = 17.5, 6.5 Hz, 2H), 2.54 (s, 1H), 1.86 (d, J = 13.0 Hz, 3H), 1.38-1.14 (m, 3H) | 462.9 | 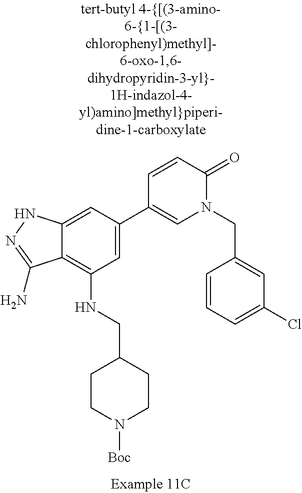 [SMILES: CC(C)(C)OC(=O)N1CCC(CNc2cc(-c3ccc(=O)n(Cc4cccc(Cl)c4)c3)cc3[nH]nc(N)c23)CC1] |
| 6G | 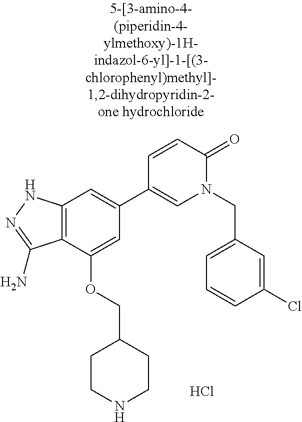 [SMILES: Nc1n[nH]c2cc(-c3ccc(=O)n(Cc4cccc(Cl)c4)c3)cc(OCC3CCNCC3)c12 ; Cl] | (400 MHz, DMSO) δ 11.50 (bs, 1H), 8.39 (s, 1H), 8.31 (s, 1H), 7.91 (dd, J = 9.5, 2.2 Hz, 1H), 7.44 (s, 1H), 7.42-7.31 (m, 3H), 6.91 (s, 1H), 6.56- 6.48 (m, 2H), 5.20 (s, 2H), 4.98 (bs, 2H), 4.05 (d, J = 4.4 Hz, 2H), 2.81 (bs, 2H), 2.12 (bs, 1H), 1.89 (d, J = 9.5 Hz, 2H), 1.50 (bs, 2H) | 463.9 | 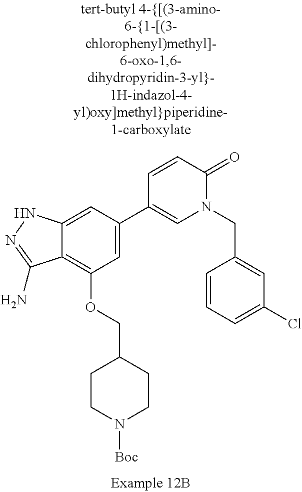 [SMILES: CC(C)(C)OC(=O)N1CCC(COc2cc(-c3ccc(=O)n(Cc4cccc(Cl)c4)c3)cc3[nH]nc(N)c23)CC1] |
| 6H | 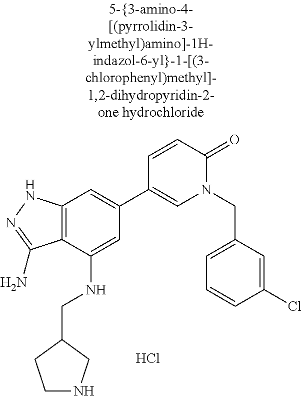 [SMILES: Nc1n[nH]c2cc(-c3ccc(=O)n(Cc4cccc(Cl)c4)c3)cc(NCC3CCNC3)c12 ; Cl] | (400 MHz, DMSO) δ 11.35 (s, 1H), 8.40 (s, 1H), 8.21 (d, J = 2.5 Hz, 1H), 7.83 (dd, J = 9.5, 2.6 Hz, 1H), 7.45 (s, 1H), 7.42- 7.31 (m, 3H), 6.56 (s, 1H), 6.51 (d, J = 7.5 Hz, 1H), 6.04 (s, 1H), 5.71 (t, 1H), 5.21 (s, 2H), 5.10 (s, 2H), 3.11-2.99 (m, 2H), 2.90 (dd, J = 11.0, 6.7 Hz, 1H), 2.71-2.59 (m, 2H), 2.03 (dd, J = 12.9, 5.3 Hz, 1H), 1.71-1.59 (m, 1H), 1.24 (s, 1H). | | 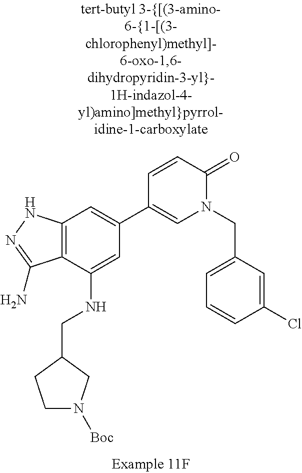 [SMILES: CC(C)(C)OC(=O)N1CCC(CNc2cc(-c3ccc(=O)n(Cc4cccc(Cl)c4)c3)cc3[nH]nc(N)c23)C1] |
| 6I | 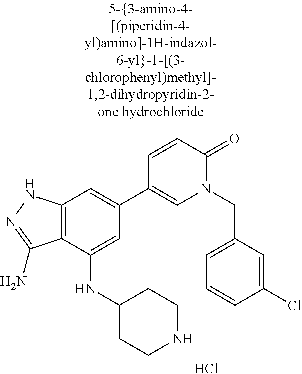 [SMILES: Nc1n[nH]c2cc(-c3ccc(=O)n(Cc4cccc(Cl)c4)c3)cc(NC3CCNCC3)c12 ; Cl] | (400 MHz, DMSO) δ 11.38 (s, 1H), 8.34 (s, 1H), 8.19 (d, J = 2.6 Hz, 1H), 7.82 (dd, J = 9.5, 2.6 Hz, 1H), 7.45 (s, 1H), 7.42- 7.33 (m, 3H), 6.56 (s, 1H), 6.52 (d, J = 9.4 Hz, 1H), 6.07 (s, 1H), 5.39 (d, J = 7.5 Hz, 1H), 5.20 (s, 2H), 3.72 (s, 1H), 3.25 (d, J = 12.5 Hz, 2H), 2.94 (t, J = 11.0 Hz, 2H), 2.10 (d, J = 10.8 Hz, 2H), 1.63 (d, J = 11.2 Hz, 2H). | | 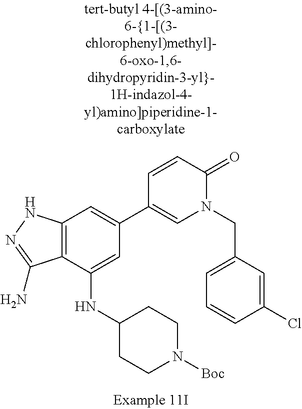 [SMILES: CC(C)(C)OC(=O)N1CCC(Nc2cc(-c3ccc(=O)n(Cc4cccc(Cl)c4)c3)cc3[nH]nc(N)c23)CC1] |

### Example 7A

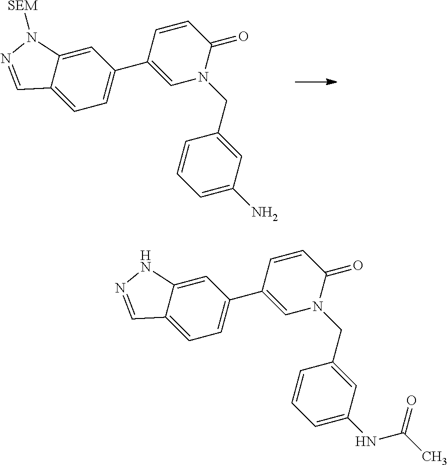 [SMILES: C[Si](C)(C)CCOCn1ncc2ccc(-c3ccc(=O)n(Cc4cccc(N)c4)c3)cc21 ; CC(=O)Nc1cccc(Cn2cc(-c3ccc4cn[nH]c4c3)ccc2=O)c1]

### N-(3-{[5-(1H-indazol-6-yl)-2-oxopyridin-1(2H)-yl]methyl}phenyl)acetamide

To a solution of 1-[(2-aminophenyl)methyl]-5-(1-{[2-(trimethylsilyl)ethoxy]methyl}-1H-indazol-6-yl)-1,2-dihydropyridin-2-one (Method 9) (0.05 g, 0.1 mmol) in dry dichloromethane (4 mL) pyridine (0.03 mL, 0.4 mmol) was added followed by acyl chloride (0.03 mL, 0.4 mmol). The reaction mixture was stirred at room temperature for 3 h and then evaporated under reduced pressure to dryness. The crude product was purified by column chromatography (silica gel; dichloromethane/methanol 95:5) to give N-(3-{[2-oxo-5-(1-{[2-(trimethylsilyl)ethoxy]methyl}-1H-indazol-6-yl)-1,2-dihydropyridin-1-yl]methyl}phenyl)acetamide (0.03 g). Then to the dissolved product (0.03 g, 0.1 mmol) in tetrahydrofuran (1.5 mL) 1.8M solution of tetra-n-butylammonium fluoride in tetrahydrofuran (2.5 mL) and 3A molecular sieves were added. The reaction mixture was stirred at 70° C. for 18 hrs. The molecular sieves were filtered off and filtrate was evaporated under reduced pressure to dryness. Purification of crude product by column chromatography (silica gel; dichloromethane/methanol 4:1) to give N-(3-{[5-(1H-indazol-6-yl)-2-oxopyridin-1(2H)-yl]methyl}phenyl)acetamide (0.01 g) as a yellowish powder; yield 45%. LC-MS (m/z) 359.3 (M+1); ¹H NMR (400 MHz, DMSO) δ 13.13 (s, 1H), 9.96 (s, 1H), 8.30 (d, J=2.6 Hz, 1H), 8.07 (s, 1H), 7.94 (dd, J=9.5, 2.7 Hz, 1H), 7.81 (d, J=8.4 Hz, 1H), 7.65 (s, 1H), 7.57 (d, J=8.2 Hz, 1H), 7.48 (s, 1H), 7.34 (dd, J=8.5, 1.4 Hz, 1H), 7.27 (t, J=7.9 Hz, 1H), 7.04 (d, J=7.6 Hz, 1H), 6.56 (d, J=9.5 Hz, 1H), 5.18 (s, 2H), 2.01 (s, 3H).

The following examples were prepared by the procedure of Example 7A, using the appropriate starting materials.

| Ex. | Product | ¹H NMR | m/z | Starting materials |
|---|---|---|---|---|
| 7B | 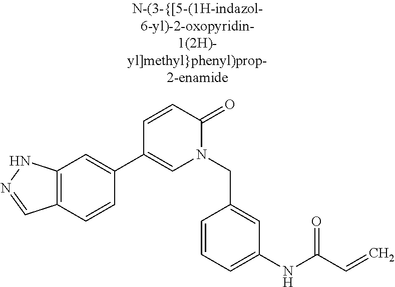 [SMILES: C=CC(=O)Nc1cccc(Cn2cc(-c3ccc4cn[nH]c4c3)ccc2=O)c1] | | 371.2 | 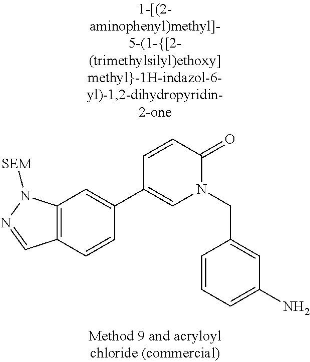 [SMILES: C[Si](C)(C)CCOCn1ncc2ccc(-c3ccc(=O)n(Cc4cccc(N)c4)c3)cc21] |

### Example 8A

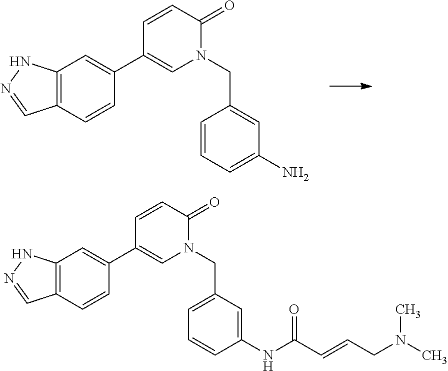

### (2Z)-4-(dimethylamino)-N-(2-{[5-(1H-indazol-6-yl)-2-oxo-1,2-dihydropyridin-1-yl]methyl}phenyl)but-2-enamide

A mixture of 1-(3-aminobenzyl)-5-(1H-indazol-6-yl)pyridin-2(1H)-one (Method 10) (0.022 g, 0.07 mmol), trans-4-dimethylaminocrotonic acid hydrochloride (0.034 g, 0.2 mmol), 4-(4,6-dimethoxy-1,3,5-triazin-2-yl)-4-methylmorpholinium chloride (0.05 g, 0.2 mmol) and pyridine (0.03 mL, 0.4 mmol) were stirred at room temperature for 4 h. After that time the solvent was evaporated under reduced pressure to dryness. The residue was purified by flash chromatography (dichloromethane/methanol 4:1) to give (2Z)-4-(dimethylamino)-N-(2-{[5-(1H-indazol-6-yl)-2-oxo-1,2-dihydropyridin-1-yl]methyl}phenyl)but-2-enamide as a brown solid (0.02 g); yield 57%. LC-MS (m/z) 428.1 (M+1); ¹H NMR (400 MHz, DMSO) δ 13.17 (s, 1H), 10.85 (s, 1H), 10.43 (s, 1H), 8.33 (d, J=2.5 Hz, 1H), 8.08 (s, 1H), 7.96 (dd, J=9.5, 2.7 Hz, 1H), 7.82 (d, J=8.5 Hz, 1H), 7.73-7.58 (m, 3H), 7.39-7.30 (m, 2H), 7.12 (d, J=7.5 Hz, 1H), 6.82-6.73 (m, 1H), 6.58 (d, J=9.5 Hz, 1H), 6.46 (d, J=15.4 Hz, 1H), 5.22 (s, 2H), 3.82 (t, J=6.1 Hz, 2H), 2.70 (s, 6H).

The following examples were prepared by the procedure of Example 8A, using the appropriate starting materials.

| | | ¹H | | |
|---|---|---|---|---|
| Ex. | Product | NMR | m/z | Starting materials |
| 8B | 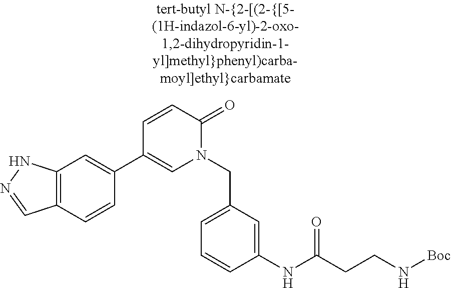 [SMILES: CC(C)(C)OC(=O)NCCC(=O)Nc1cccc(Cn2cc(-c3ccc4cn[nH]c4c3)ccc2=O)c1] | | | 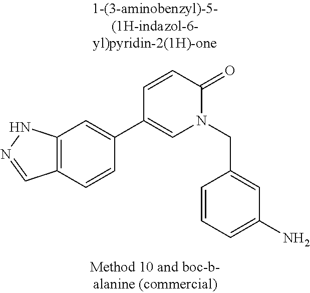 [SMILES: Nc1cccc(Cn2cc(-c3ccc4cn[nH]c4c3)ccc2=O)c1] |

### Example 9

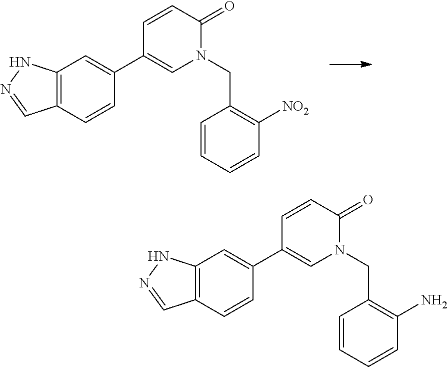 [SMILES: O=c1ccc(-c2ccc3cn[nH]c3c2)cn1Cc1ccccc1[N+](=O)[O-] ; Nc1ccccc1Cn1cc(-c2ccc3cn[nH]c3c2)ccc1=O]

### 1-(2-aminobenzyl)-5-(1H-indazol-6-yl)pyridin-2(1H)-one

A mixture of 5-(1H-indazol-6-yl)-1-(2-nitrobenzyl)pyridin-2(1H)-one (Example 2C) (0.1 g, 0.3 mmol) and tin (II) chloride (0.3 g, 1.4 mmol) in ethyl acetate were heated at reflux for 30 min. After cooled to the ambient temperature water was added followed by 1M aqueous solution of hydrochloride. Water phase was extracted with ethyl acetate. Combined organic layers were washed with brine, dried over anhydrous magnesium sulfate and concentrated under reduced pressure. The crude product was purified by column chromatography (silica gel; dichloromethane/methanol 4:1) to give 1-(2-aminobenzyl)-5-(1H-indazol-6-yl)pyridin-2(1H)-one (0.02 g); yield 50%. LC-MS (m/z) 317.4 (M+1).

### Example 10

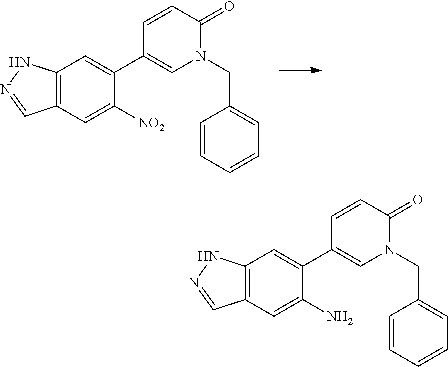 [SMILES: O=c1ccc(-c2cc3[nH]ncc3cc2[N+](=O)[O-])cn1Cc1ccccc1 ; Nc1cc2cn[nH]c2cc1-c1ccc(=O)n(Cc2ccccc2)c1]

### 5-(5-amino-1H-indazol-6-yl)-1-benzylpyridin-2(1H)-one

A 1-benzyl-5-(5-nitro-1H-indazol-6-yl)pyridin-2(1H)-one (Example 2Z) (0.04 g, 0.12 mmol) was dissolved in ethanol (3 mL), then Raney's nickel was added followed by hydrazine monohydrate (0.06 mL, 1.1 mmol). The reaction mixture was stirred at room temperature for 40 min., filtered through Celite and washed with methanol. Filtrate was concentrated under reduced pressure and crude product was purified by preparative HPLC to give 5-(5-amino-1H-indazol-6-yl)-1-benzylpyridin-2(1H)-one (0.01 g); yield 27%. LC-MS (m/z) 317.1 (M+1).

### Example 11A

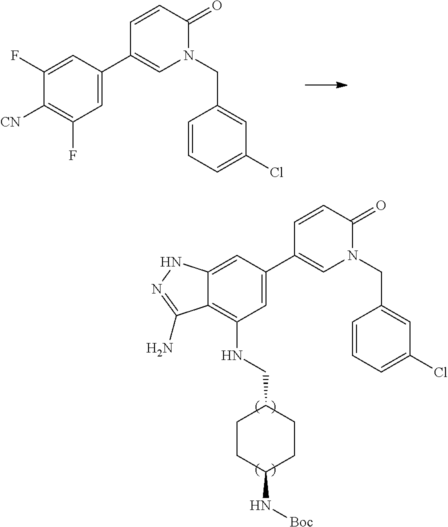

### tert-butyl N-[trans-4-{[(3-amino-6-{1-[(3-chlorophenyl)methyl]-6-oxo-1,6-dihydropyridin-3-yl}-1H-indazol-4-yl)amino]methyl}cyclohexyl]carbamate

A 4-{1-[(3-chlorophenyl)methyl]-6-oxo-1,6-dihydropyridin-3-yl}-2,6-difluorobenzonitrile (Example 2AZ) (0.13 g, 0.23 mmol), trans-4-(boc-amino)cyclohexylmethylamine (0.09 g, 0.4 mmol), DIPEA (0.8 mL, 0.5 mmol) were suspended in 1,4-dioxane (1 mL). The reaction mixture was heated at 90° C. until the starting material was consumed. Then hydrazine monohydrate wad added and reaction mixture was heated under reflux for next 24 h. After cooled to room temperature water was added and water phase was extracted with ethyl acetate. Combined organic phases were dried over sodium sulfate and concentrated under reduced pressure. The crude product was purified by flash chromatography (ethyl acetate/methanol 10%) to give tert-butyl N-[trans-4-{[(3-amino-6-{1-[(3-chlorophenyl)methyl]-6-oxo-1,6-dihydropyridin-3-yl}-1H-indazol-4-yl)amino]methyl}cyclohexyl]carbamate (0.07 g); yield 54%. LC-MS (m/z) 577.2 (M+1).

The following examples were prepared by the procedure of Example 11A, using the appropriate starting materials.

| Ex. | Product | ¹H NMR | m/z | Starting materials |
|---|---|---|---|---|
| 11B | 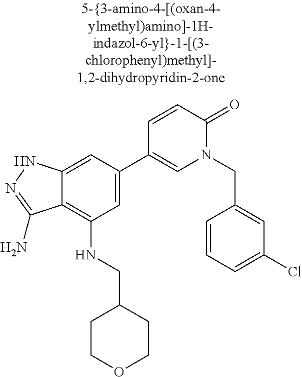 [SMILES: Nc1n[nH]c2cc(-c3ccc(=O)n(Cc4cccc(Cl)c4)c3)cc(NCC3CCOCC3)c12] | (400 MHz, DMSO) δ 11.32 (s, 1H), 8.18 (d, J = 2.6 Hz, 1H), 7.82 (dd, J = 9.5, 2.6 Hz, 1H), 7.45 (s, 1H), 7.42- 7.31 (m, 3H), 6.52 (d, J = 8.6 Hz, 2H), 5.98 (s, 1H), 5.59 (t, J = 5.6 Hz, 1H), 5.20 (s, 2H), 5.03 (s, 2H), 3.88 (dd, J = 11.3, 2.8 Hz, 2H), 3.29 (d, J = 13.2 Hz, 2H), 3.10 (t, J = 6.2 Hz, 2H), 1.99-1.86 (m, 1H), 1.72 (d, J = 12.8 Hz, 2H), 1.27 (qd, J = 12.1, 4.5 Hz, 2H) | 463.9 | 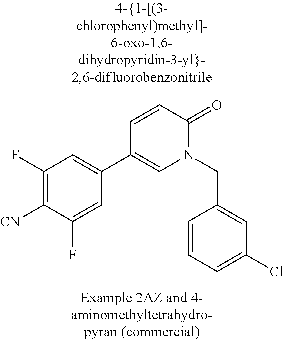 |
| 11C | 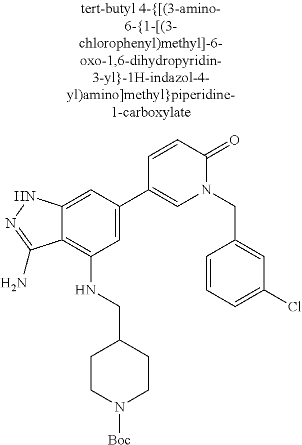 [SMILES: CC(C)(C)OC(=O)N1CCC(CNc2cc(-c3ccc(=O)n(Cc4cccc(Cl)c4)c3)cc3[nH]nc(N)c23)CC1] | | 562.9 | 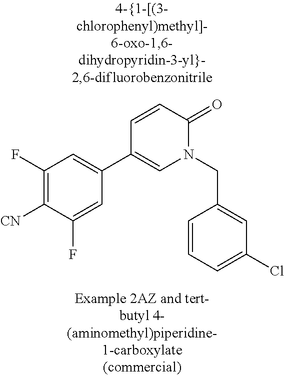 |
| 11D | 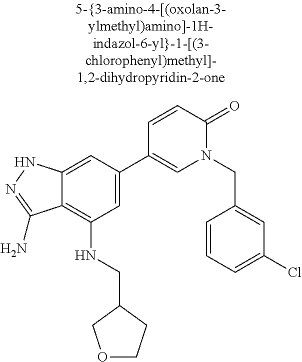 [SMILES: Nc1n[nH]c2cc(-c3ccc(=O)n(Cc4cccc(Cl)c4)c3)cc(NCC3CCOC3)c12] | (400 MHz, DMSO) δ 11.33 (s, 1H), 8.20 (d, J = 2.5 Hz, 1H), 7.83 (dd, J = 9.5, 2.7 Hz, 1H), 7.45 (bs, 1H), 7.41- 7.28 (m, 3H), 6.57- 6.48 (m, 2H), 6.02 (s, 1H), 5.63 (t, J = 5.4 Hz, 1H), 5.20 (s, 2H), 5.06 (s, 2H), 3.85- 3.73 (m, 2H), 3.66 (dd, J = 15.0, 7.8 Hz, 1H), 3.55 (dd, J = 8.5, 5.3 Hz, 1H), 3.24-3.12 (m, 2H), 2.71-2.59 (m, 1H), 2.09-1.97 (m, 1H), 1.73-1.60 (m, 1H) | 449.9 | 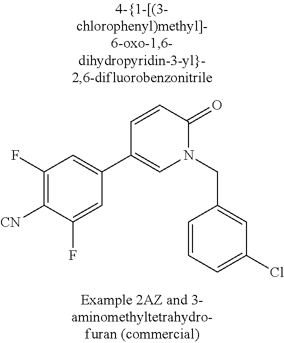 |
| 11F | 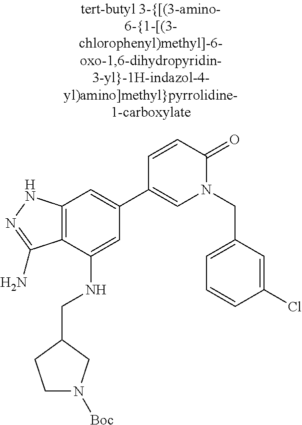 [SMILES: CC(C)(C)OC(=O)N1CCC(CNc2cc(-c3ccc(=O)n(Cc4cccc(Cl)c4)c3)cc3[nH]nc(N)c23)C1] | | | 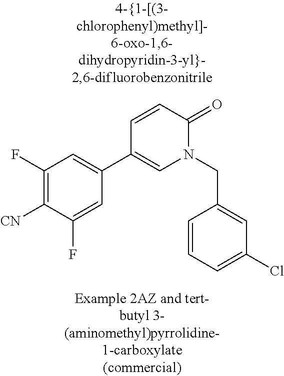 |
| 11G | 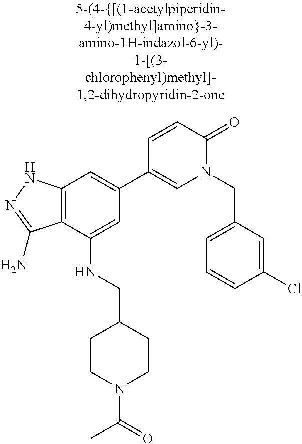 [SMILES: CC(=O)N1CCC(CNc2cc(-c3ccc(=O)n(Cc4cccc(Cl)c4)c3)cc3[nH]nc(N)c23)CC1] | (400 MHz, DMSO) δ 11.32 (s, 1H), 8.18 (d, J = 2.6 Hz, 1H), 7.82 (dd, J = 9.5, 2.6 Hz, 1H), 7.45 (s, 1H), 7.42- 7.32 (m, 3H), 6.52 (d, J = 8.3 Hz, 2H), 5.98 (s, 1H), 5.61 (t, J = 5.6 Hz, 1H), 5.20 (s, 2H), 5.04 (s, 2H), 4.38 (d, J = 12.7 Hz, 1H), 3.83 (d, J = 14.0 Hz, 1H), 3.11 (t, J = 6.2 Hz, 2H), 2.99 (t, J = 11.3 Hz, 1H), 1.99 (s, 3H), 1.93 (s, 1H), 1.82 (t, J = 15.6 Hz, 2H), 1.26-0.99 (m, 3H). | | 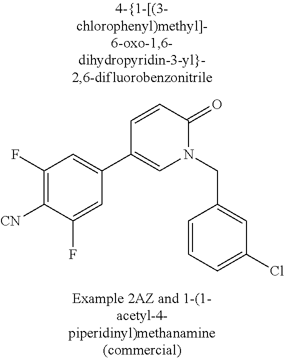 |
| 11I | 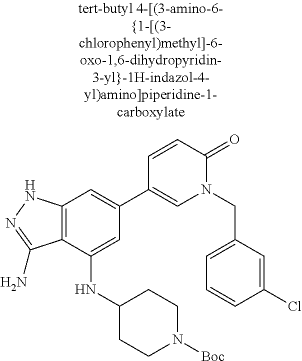 [SMILES: CC(C)(C)OC(=O)N1CCC(Nc2cc(-c3ccc(=O)n(Cc4cccc(Cl)c4)c3)cc3[nH]nc(N)c23)CC1] | | | 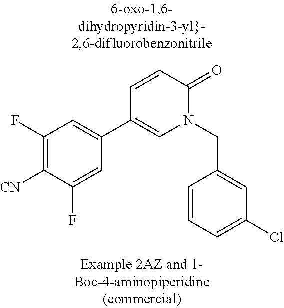 |
| 11J | 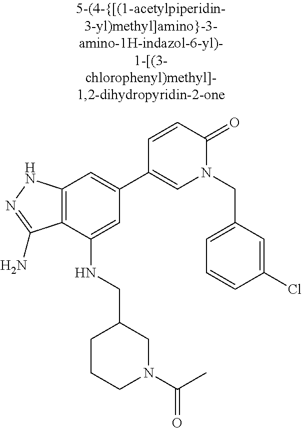 [SMILES: CC(=O)N1CCCC(CNc2cc(-c3ccc(=O)n(Cc4cccc(Cl)c4)c3)cc3[nH]nc(N)c23)C1] | (400 MHz, DMSO) δ 11.34 (s, 1H), 8.19 (dd, J = 6.1, 2.5 Hz, 1H), 7.82 (ddd, J = 9.2, 6.5, 2.6 Hz, 1H), 7.47-7.30 (m, 4H), 6.53 (t, J = 10.7 Hz, 2H), 5.98 (d, J = 16.3 Hz, 1H), 5.63 (dd, J = 17.3, 11.4 Hz, 1H), 5.21 (d, J = 2.6 Hz, 2H), 5.06 (s, 2H), 3.97 (ddd, J = 77.7, 76.9, 12.0 Hz, 2H), 3.97 (ddd, J = 77.7, 76.9, 12.0 Hz, 2H), 3.17-3.03 (m, 3H), 3.20-3.00 (m, 3H), 3.00-2.70 (m, 1H), 1.98 (d, J = 8.5 Hz, 3H), 1.76 (dd, J = 77.0, 31.7 Hz, 3H), 1.48-1.12 (m, 3H). | | 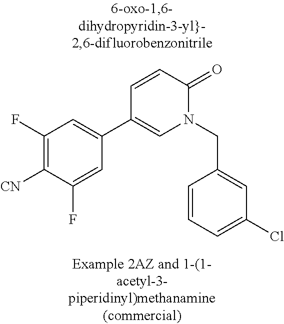 |

### Example 12A

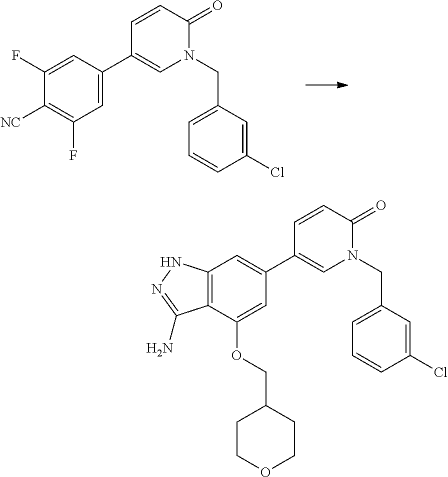

### 5-[3-amino-4-(oxan-4-ylmethoxy)-1H-indazol-6-yl]-1-[(3-chlorophenyl)methyl]-1,2-dihydropyridin-2-one

To a solution of tetrahydro-2H-pyran-4-ylmethanol (0.021 g, 0.18 mmol) in THF (1 mL) cooled to 0° C., NaH (60% dispersion in oil) (0.009 g, 0.22 mmol) was added. The mixture was stirred for 30 min at 0° C. before adding a solution of 4-{1-[(3-chlorophenyl)methyl]-6-oxo-1,6-dihydropyridin-3-yl}-2,6-difluorobenzonitrile (Example 2AZ) (0.065 g, 0.18 mmol) in THF (1 mL). The reaction mixture was allowed to warm up to room temperature and then was heated at 50° C. overnight. Next hydrazine monohydrate (0.45 g, 9.1 mmol) was added and heating was continued at 60° C. overnight. After cooled to the room temperature water was added and water phase was extracted with ethyl acetate. Combined organic phases were washed with brine, dried over sodium sulfate and concentrated under reduced pressure. The crude product was purified by flash chromatography (DCM/methanol 7%) to give 5-[3-amino-4-(oxan-4-ylmethoxy)-1H-indazol-6-yl]-1-[(3-chlorophenyl)methyl]-1,2-dihydropyridin-2-one (0.049 g); yield 57%. LC-MS (m/z) 465.2 (M+1). ¹H NMR (400 MHz, DMSO) δ 11.48 (s, 1H), 8.30 (d, J=2.6 Hz, 1H), 7.91 (dd, J=9.5, 2.7 Hz, 1H), 7.44 (s, 1H), 7.42-7.29 (m, 3H), 6.91 (d, J=0.7 Hz, 1H), 6.54 (d, J=9.5 Hz, 1H), 6.49 (s, 1H), 5.20 (s, 2H), 4.94 (s, 2H), 4.03 (d, J=6.3 Hz, 2H), 3.91 (dd, J=11.1, 3.3 Hz, 2H), 3.38 (t, J=10.8 Hz, 2H), 2.15 (s, 1H), 1.75 (s, 3H), 1.39 (qd, J=12.2, 4.3 Hz, 2H).

The following examples were prepared by the procedure of Example 12A, using the appropriate starting materials.

| Ex. | Product | ¹H NMR | m/z | Starting materials |
|---|---|---|---|---|
| 12B | 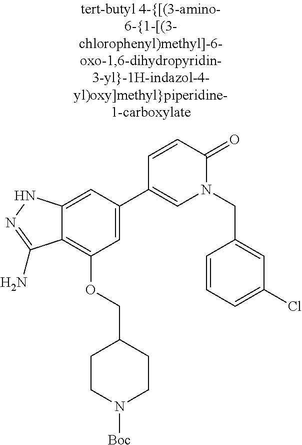 [SMILES: CC(C)(C)OC(=O)N1CCC(COc2cc(-c3ccc(=O)n(Cc4cccc(Cl)c4)c3)cc3[nH]nc(N)c23)CC1] | | 563.9 | 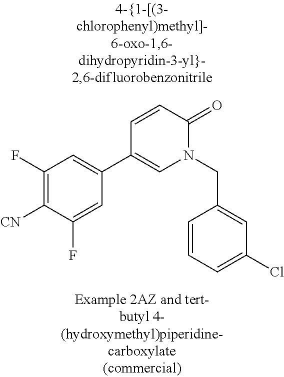 |
| 12C | 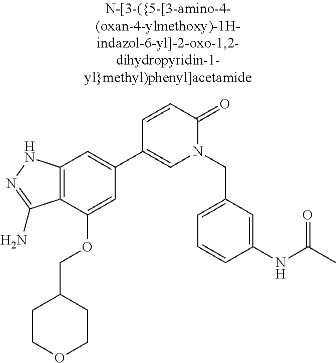 [SMILES: CC(=O)Nc1cccc(Cn2cc(-c3cc(OCC4CCOCC4)c4c(N)n[nH]c4c3)ccc2=O)c1] | (400 MHz, DMSO) δ 11.47 (s, 1H), 9.95 (s, 1H), 8.22 (d, J = 2.6 Hz, 1H), 7.90 (dd, J = 9.5, 2.7 Hz, 1H), 7.54 (d, J = 8.0 Hz, 1H), 7.48 (s, 1H), 7.26 (t, J = 7.9 Hz, 1H), 7.01 (d, J = 8.0 Hz, 1H), 6.89 (s, 1H), 6.53 (d, J = 9.4 Hz, 1H), 6.49 (s, 1H), 5.18 (s, 2H), 4.93 (s, 2H), 4.03 (d, J = 6.3 Hz, 2H), 3.91 (d, J = 8.2 Hz, 2H), 3.39 (d, J = 11.6 Hz, 2H), 2.14 (s, 1H), 2.01 (s, 3H), 1.75 (d, J = 12.4 Hz, 2H), 1.39 (d, J = 8.5 Hz, 2H). | | 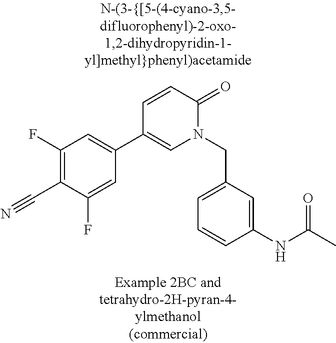 [SMILES: CC(=O)Nc1cccc(Cn2cc(-c3cc(F)c(C#N)c(F)c3)ccc2=O)c1] |

### Methods:

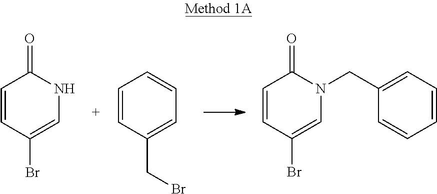 [SMILES: O=c1ccc(Br)c[nH]1 ; BrCc1ccccc1 ; O=c1ccc(Br)cn1Cc1ccccc1]

### 1-benzyl-5-bromopyridin-2(1H)-one

5-Bromopyridin-2-ol (3 g, 17.2 mmol) was suspended in dry DMF (20 mL), cooled to 0° C. then sodium hydride (60% in oil) (0.7 g, 18.9 mmol) was added in portions. The reaction mixture was stirred for 20 min then benzyl bromide (2.7 mL, 18.9 mmol) was dropped. The stirring was continues overnight at room temperature. To the reaction mixture water was added and water phase was extracted with ethyl acetate. Combined organic phases were washed with brine, dried over sodium sulfate and concentrated under reduced pressure. The crude product was purified by column chromatography (silica gel, dichlorometane/methanol 9:1) to give 1-benzyl-5-bromopyridin-2(1H)-one as an yellowish solid (3.8 g); yield 83%. LC-MS (m/z) 265.8 (M+1). ¹H NMR (400 MHz, DMSO) δ 8.17 (dd, J=2.8, 0.4 Hz, 1H), 7.55 (dd, J=9.7, 2.8 Hz, 1H), 7.38-7.27 (m, 5H), 6.43-6.40 (m, 1H), 5.07 (s, 2H).

The following compounds were prepared by the procedure of Method 1A, using the appropriate starting materials.

| Method | Product | ¹H NMR | m/z | Starting material |
|---|---|---|---|---|
| 1B | 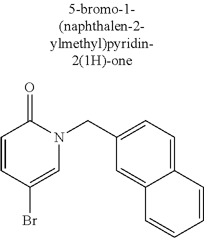 [SMILES: O=c1ccc(Br)cn1Cc1ccc2ccccc2c1] | (400 MHz, DMSO) δ 8.17-8.10 (m, 1H), 8.03- 7.95 (m, 2H), 7.92 (t, J = 8.1 Hz, 1H), 7.65- 7.54 (m, 3H), 7.49 (dd, J = 8.2, 7.2 Hz, 1H), 7.16 (dd, J = 7.1, 0.9 Hz, 1H), 6.52 (dd, J = 9.7, 0.4 Hz, 1H), 5.58 (s, 2H) | 317.0 | 5-bromopyridin-2-ol (commercial) and 2- (bromomethyl)naphthalene (commercial) |
| 1C | 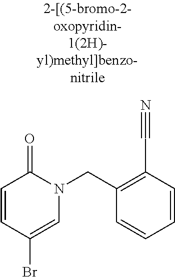 [SMILES: N#Cc1ccccc1Cn1cc(Br)ccc1=O] | (400 MHz, DMSO) δ 8.21 (d, J = 2.7 Hz, 1H), 7.88 (dd, J = 7.7, 0.8 Hz, 1H), 7.73- 7.59 (m, 2H), 7.55-7.46 (m, 1H), 7.14 (d, J = 7.8 Hz, 1H), 6.44 (d, J = 9.7 Hz, 1H), 5.26 (s, 2H) | 290.0 | 5-bromopyridin-2-ol (commercial) and 2- (bromomethyl)benzo- nitryle (commercial) |
| 1D | 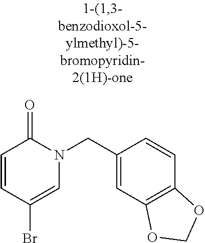 [SMILES: O=c1ccc(Br)cn1Cc1ccc2c(c1)OCO2] | (300 MHz, DMSO) δ 8.11 (dd, J = 2.8, 0.5 Hz, 1H), 7.49 (dd, J = 9.7, 2.8 Hz, 1H), 6.94 (d, J = 1.0 Hz, 1H), 6.85 (d, J = 0.7 Hz, 2H), 6.42-6.32 (m, 1H), 5.97 (s, 2H), 4.93 (s, 2H) | 309.0 | 5-bromopyridin-2-ol (commercial) and 5- (bromomethyl)-1,3- benzodioxole (commercial) |
| 1F | 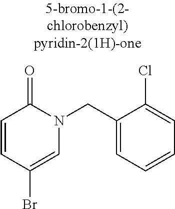 [SMILES: O=c1ccc(Br)cn1Cc1ccccc1Cl] | (300 MHz, DMSO) δ 8.04 (d, J = 2.8 Hz, 1H), 7.60 (dd, J = 9.7, 2.8 Hz, 1H), 7.54- 7.45 (m, 1H), 7.41-7.21 (m, 2H), 6.94- 6.81 (m, 1H), 6.44 (d, J = 9.7 Hz, 1H), 5.12 (s, 2H) | 299.0 | 5-bromopyridin-2-ol (commercial) and 2- chlorobenzylbromide (commercial) |
| 1G | 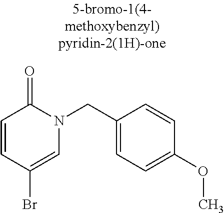 [SMILES: COc1ccc(Cn2cc(Br)ccc2=O)cc1] | (400 MHz, DMSO-d6) δ 8.13 (d, J = 3.2 Hz, 1H), 7.51 (dd, J = 9.7, 2.8 Hz, 1H), 7.36- 7.28 (m, 2H), 6.95-6.88 (m, 2H), 6.40 (d, J = 9.7 Hz, 1H), 4.99 (s, 2H), 3.73 (s, 3H). | 295.9 | 5-bromopyridin-2-ol (commercial) and 4- methoxybenzyl chloride (commercial) |
| 1H | 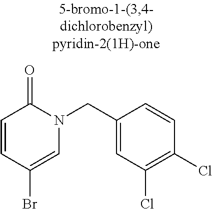 [SMILES: O=c1ccc(Br)cn1Cc1ccc(Cl)c(Cl)c1] | (300 MHz, DMSO) δ 8.20 (d, J = 2.8 Hz, 1H), 7.63-7.57 (m, 2H), 7.54 (dd, J = 9.7, 2.8 Hz, 1H), 7.29 (dd, J = 8.3, 2.1 Hz, 1H), 6.40 (d, J = 9.7 Hz, 1H), 5.02 (s, 2H) | 333.9 | 5-bromopyridin-2-ol (commercial) and 3,4- dichlorobenzylbromide (commercial) |
| 1I | 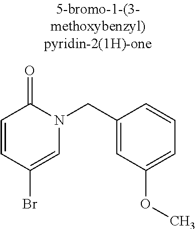 [SMILES: COc1cccc(Cn2cc(Br)ccc2=O)c1] | (400 MHz, DMSO) δ 8.15 (d, J = 2.7 Hz, 1H), 7.55 (dd, J = 9.7, 2.8 Hz, 1H), 7.27 (t, J = 7.9 Hz, 1H), 6.95- 6.82 (m, 3H), 6.43 (d, J = 9.7 Hz, 1H), 5.04 (s, 2H), 3.74 (s, 3H) | 295.9 | 5-bromopyridin-2-ol (commercial) and 3- methoxybenzyl chloride (commercial) |
| 1J | 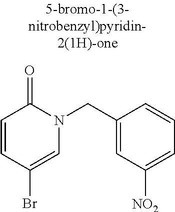 [SMILES: O=c1ccc(Br)cn1Cc1cccc([N+](=O)[O-])c1] | | 310.8 | 5-bromopyridin-2-ol (commercial) and 3- nitrobenzyl bromide (commercial) |
| 1K | 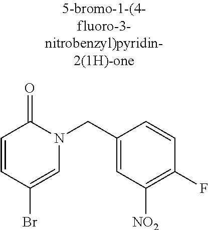 [SMILES: O=c1ccc(Br)cn1Cc1ccc(F)c([N+](=O)[O-])c1] | (300 MHz, DMSO) δ 8.26 (dd, J = 2.8, 0.5 Hz, 1H), 8.18 (dd, J = 7.3, 2.3 Hz, 1H), 7.78 (ddd, J = 8.6, 4.4, 2.3 Hz, 1H), 7.60- 7.39 (m, 2H), 6.41 (d, J = 9.7 Hz, 1H), 5.10 (s, 2H) | 328.7 | 5-bromopyridin-2-ol (commercial) and 4- fluoro-3-nitrobenzyl bromide (commercial) |
| 1M | 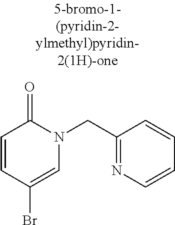 [SMILES: O=c1ccc(Br)cn1Cc1ccccn1] | (400 MHz, DMSO-d6) δ 8.51 (ddd, J = 4.8, 1.7, 0.9 Hz, 1H), 8.14 (d, J = 2.8 Hz, 1H), 7.78 (td, J = 7.7, 1.8 Hz, 1H), 7.57 (dd, J = 9.7, 2.8 Hz, 1H), 7.34- 7.24 (m, 2H), 6.40 (d, J = 9.7 Hz, 1H), 5.17 (s, 2H) | 266.1 | 5-bromopyridin-2-ol (commercial) and 2- (bromomethyl)pyridine hydrobromide |
| 1N | 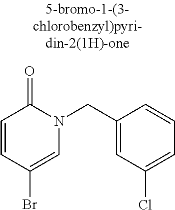 [SMILES: O=c1ccc(Br)cn1Cc1cccc(Cl)c1] | (400 MHz, DMSO) δ 8.22 (d, J = 2.8 Hz, 1H), 7.56 (dd, J = 9.7, 2.8 Hz, 1H), 7.44- 7.34 (m, 3H), 7.29 (dt, J = 4.1, 3.6 Hz, 1H), 6.43 (d, J = 9.7 Hz, 1H), 5.06 (s, 2H) | 299.8 | 5-bromopyridin-2-ol (commercial) and 3- chlorobenzyl bromide (commercial) |
| 1O | 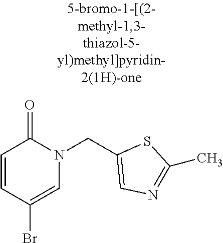 | | 286.1 | 5-bromopyridin-2-ol (commercial) and 4- chloromethyl-2- methylthiazole (commercial) |
| 1S | 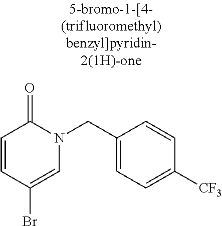 [SMILES: O=c1ccc(Br)cn1Cc1ccc(C(F)(F)F)cc1] | (400 MHz, DMSO-d6) δ 8.23 (d, J = 2.7 Hz, 1H), 7.73 (d, J = 8.1 Hz, 2H), 7.58 (dd, J = 9.7, 2.8 Hz, 1H), 7.51 (d, J = 8.0 Hz, 2H), 6.44 (d, J = 9.7 Hz, 1H), 5.16 (s, 2H). | 347.6 | 5-bromopyridin-2-ol (commercial) and 4- trifluoromethylbenzyl- chloride (commercial) |
| 1U | 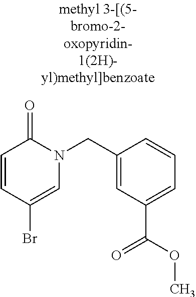 [SMILES: COC(=O)c1cccc(Cn2cc(Br)ccc2=O)c1] | | 323.6 | 5-bromopyridin-2-ol (commercial) and 3- bromomethyl benzoic acid methyl ester (commercial) |
| 1W | 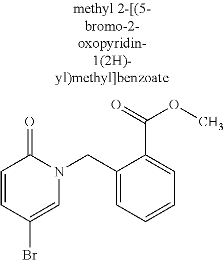 [SMILES: COC(=O)c1ccccc1Cn1cc(Br)ccc1=O] | | 323.8 | 5-bromopyridin-2-ol (commercial) and 2- bromomethyl benzoic acid methyl ester (commercial) |
| 1X | 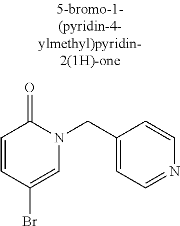 [SMILES: O=c1ccc(Br)cn1Cc1ccncc1] | | 266.8 | 5-bromopyridin-2-ol (commercial) and (4- bromomethyl)pyridine hydrobromide (commercial) |
| 1Y | 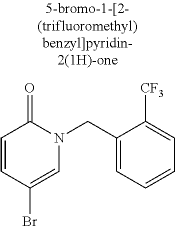 [SMILES: O=c1ccc(Br)cn1Cc1ccccc1C(F)(F)F] | (400 MHz, DMSO-d6) δ 8.12 (d, J = 2.8 Hz, 1H), 7.79 (d, J = 7.7 Hz, 1H), 7.70-7.59 (m, 2H), 7.51 (t, J = 7.6 Hz, 1H), 6.88 (d, J = 7.8 Hz, 1H), 6.49 (d, J = 9.7 Hz, 1H), 5.29 (s, 2H). | 333.9 | 5-bromopyridin-2-ol (commercial) and 2- trifluoromethylbenzyl bromide (commercial) |
| 1Z | 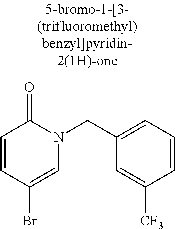 [SMILES: O=c1ccc(Br)cn1Cc1cccc(C(F)(F)F)c1] | (400 MHz, DMSO-d6) δ 8.27 (d, J = 2.8 Hz, 1H), 7.73 (s, 1H), 7.72-7.59 (m, 3H), 7.57 (dd, J = 9.7, 2.8 Hz, 1H), 6.44 (d, J = 9.7 Hz, 1H), 5.15 (s, 2H). | 333.9 | 5-bromopyridin-2-ol (commercial) and 3- trifluoromethylbenzyl bromide (commercial) |
| 1AA | 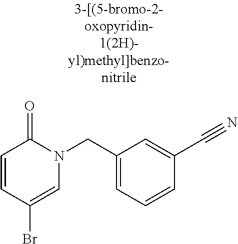 [SMILES: N#Cc1cccc(Cn2cc(Br)ccc2=O)c1] | | 290.8 | 5-bromopyridin-2-ol (commercial) and 3- (bromomethyl)benzo- nitryle (commercial) |
| 1AB | 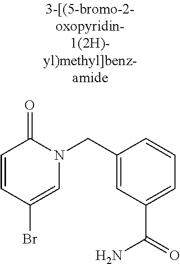 [SMILES: NC(=O)c1cccc(Cn2cc(Br)ccc2=O)c1] | (400 MHz, DMSO-d6) δ 8.19 (d, J = 2.8 Hz, 1H), 7.98 (s, 1H), 7.83-7.77 (m, 2H), 7.56 (dd, J = 9.7, 2.8 Hz, 1H), 7.47-7.36 (m, 3H), 6.43 (d, J = 9.7 Hz, 1H), 5.11 (s, 2H). | 308.0 | 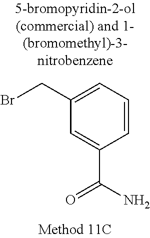 [SMILES: NC(=O)c1cccc(CBr)c1] |
| 1AC | 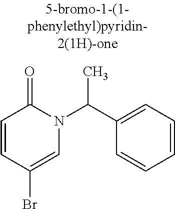 [SMILES: CC(c1ccccc1)n1cc(Br)ccc1=O] | (400 MHz, DMSO-d6) δ 7.84 (d, J = 2.7, 1H), 7.50 (dd, J = 9.7, 2.8 Hz, 1H), 7.46-7.25 (m, 5H), 6.42 (d, J = 9.7 Hz, 1H), 6.12 (q, J = 7.2 Hz, 1H), 1.72 (d, J = 7.2 Hz, 3H) | 279.0 | 5-bromopyridin-2-ol (commercial) and 1- (bromoethyl)benzene (commercial) |
| 1AD | 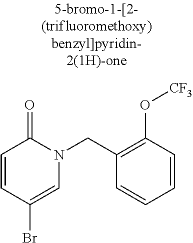 [SMILES: O=c1ccc(Br)cn1Cc1ccccc1OC(F)(F)F] | | 349.0 | 5-bromopyridin-2-ol (commercial) and 2- (trifluoromethoxy) benzyl bromide (commercial) |
| 1AE | 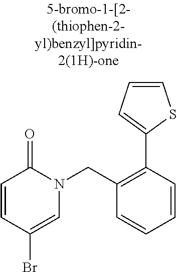 [SMILES: O=c1ccc(Br)cn1Cc1ccccc1-c1cccs1] | (400 MHz, DMSO-d6) δ 7.81 (d, J = 2.8 Hz, 1H), 7.67 (dd, J = 5.1, 1.2 Hz, 1H), 7.57 (dd, J = 9.7, 2.8 Hz, 1H), 7.46-7.40 (m, 1H), 7.40- 7.34 (m, 2H), 7.25 (dd, J = 3.5, 1.1 Hz, 1H), 7.19 (dd, J = 5.1, 3.5 Hz, 1H), 6.92 (dd, J = 5.5, 3.6 Hz, 1H), 6.41 (d, J = 9.7 Hz, 1H), 5.19 (s, 2H). | 347.8 | 5-bromopyridin-2-ol (commercial) and 2- [2- (bromomethyl)phenyl] thiophene (commercial) |
| 1AG | 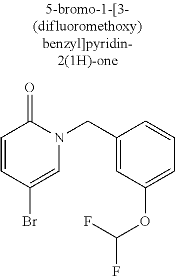 [SMILES: O=c1ccc(Br)cn1Cc1cccc(OC(F)F)c1] | | 331.8 | 5-bromopyridin-2-ol (commercial) and 3- (difluoromethoxy) benzyl bromide (commercial) |
| 1AH | 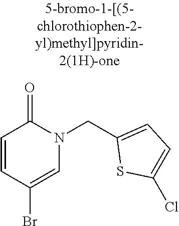 [SMILES: O=c1ccc(Br)cn1Cc1ccc(Cl)s1] | (400 MHz, DMSO-d6) δ 8.22 (d, J = 2.8 Hz, 1H), 7.55 (dd, J = 9.7, 2.8 Hz, 1H), 7.10 (d, J = 3.8 Hz, 1H), 6.99 (d, J = 3.8, Hz, 1H), 6.44 (d, J = 9.7 Hz, 1H), 5.13 (s, 2H). | 305.8 | 5-bromopyridin-2-ol (commercial) and 2- chloro-5- (chloromethyl)thiophene (commercial) |
| 1AI | 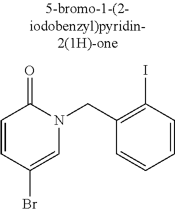 [SMILES: O=c1ccc(Br)cn1Cc1ccccc1I] | (400 MHz, DMSO-d6) δ 8.04 (d, J = 2.7 Hz, 1H), 7.93 (dd, J = 7.8, 1.2 Hz, 1H), 7.64 (dd, J = 9.7, 2.8 Hz, 1H), 7.38 (td, J = 7.6, 1.2 Hz, 1H), 7.08 (td, J = 7.7, 1.6 Hz, 1H), 6.71 (dd, J = 7.7, 1.3 Hz, 1H), 6.48 (d, J = 9.7 Hz, 1H), 5.02 (s, 2H). | 391.6 | 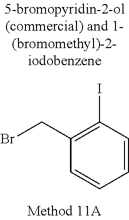 |
| 1AJ | 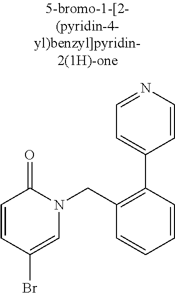 [SMILES: O=c1ccc(Br)cn1Cc1ccccc1-c1ccncc1] | (400 MHz, DMSO-d6) δ 8.72-8.65 (m, 2H), 7.83 (d, J = 2.8 Hz, 1H), 7.54 (dd, J = 9.7, 2.8 Hz, 1H), 7.46-7.39 (m, 4H), 7.32- 7.27 (m, 1H), 7.01 (dd, J = 5.1, 3.8 Hz, 1H), 6.36 (d, J = 9.7 Hz, 1H), 5.05 (s, 2H). | 342.8 | 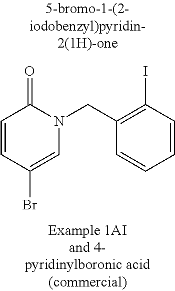 [SMILES: O=c1ccc(Br)cn1Cc1ccccc1I] |
| 1AK | 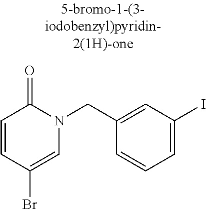 [SMILES: O=c1ccc(Br)cn1Cc1cccc(I)c1] | (400 MHz, DMSO-d6) δ 8.21 (d, J = 2.8 Hz, 1H), 7.78-7.71 (m, 1H), 7.70- 7.64 (m, 1H), 7.56 (dd, J = 9.7, 2.8 Hz, 1H), 7.33 (d, J = 7.7 Hz, 1H), 7.16 (t, J = 7.8 Hz, 1H), 6.42 (d, J = 9.7 Hz, 1H), 5.02 (s, 2H). | 391.7 | 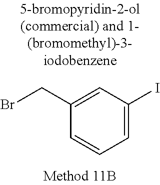 |
| 1AL | 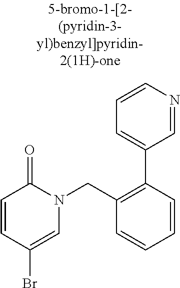 [SMILES: O=c1ccc(Br)cn1Cc1ccccc1-c1cccnc1] | | 342.0 | 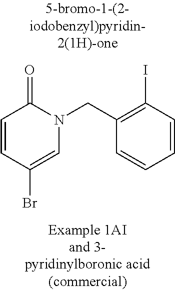 [SMILES: O=c1ccc(Br)cn1Cc1ccccc1I] |
| 1AM | 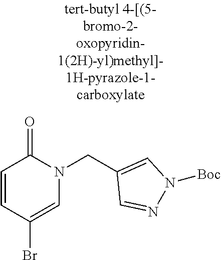 [SMILES: CC(C)(C)OC(=O)n1cc(Cn2cc(Br)ccc2=O)cn1] | | 355.7 | 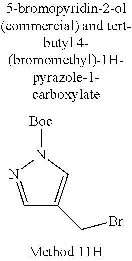 [SMILES: CC(C)(C)OC(=O)n1cc(CBr)cn1] |
| 1AO | 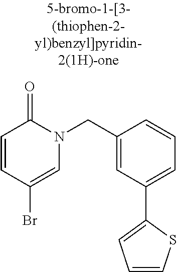 [SMILES: O=c1ccc(Br)cn1Cc1cccc(-c2cccs2)c1] | (400 MHz, DMSO-d6) δ 8.69-8.63 (m, 2H), 8.25 (d, J = 2.8 Hz, 1H), 7.82 (s, 1H), 7.74 (d, J = 7.8 Hz, 1H), 7.71- 7.64 (m, 2H), 7.55 (dd, J = 9.7, 2.8 Hz, 1H), 7.51 (t, J = 7.7 Hz, 1H), 7.41 (d, J = 7.3 Hz, 1H), 6.43 (d, J = 9.7 Hz, 1H), 5.15 (s, 2H). | 347.6 | 5-bromopyridin-2-ol (commercial) and 2- [3- (bromomethyl)phenyl] thiophene (commercial) |
| 1AP | 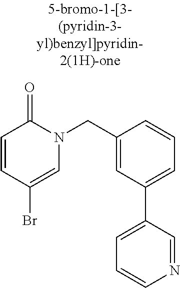 [SMILES: O=c1ccc(Br)cn1Cc1cccc(-c2cccnc2)c1] | | 342.0 | 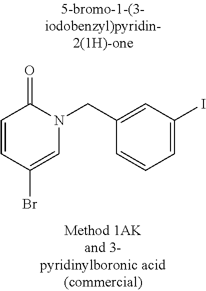 [SMILES: O=c1ccc(Br)cn1Cc1cccc(I)c1] |
| 1AR | 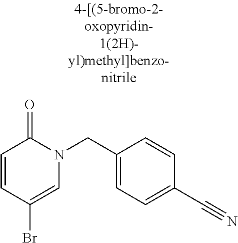 [SMILES: N#Cc1ccc(Cn2cc(Br)ccc2=O)cc1] | | 290.6 | 5-bromopyridin-2-ol (commercial) and 4- cyanobenzyl bromide (commercial) |
| 1AU | 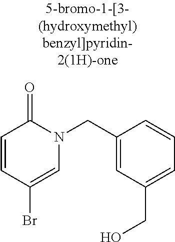 [SMILES: O=c1ccc(Br)cn1Cc1cccc(CO)c1] | | 295.7 | 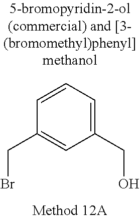 |
| 1AW | 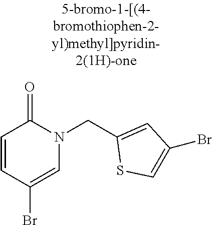 [SMILES: O=c1ccc(Br)cn1Cc1cc(Br)cs1] | (400 MHz, DMSO-d6) δ 8.22 (d, J = 2.6 Hz, 1H), 7.62-7.57 (m, 1H), 7.55 (dd, J = 9.7, 2.8 Hz, 1H), 7.25-7.20 (m, 1H), 6.43 (d, J = 9.7 Hz, 1H), 5.19 (s, 2H). | | 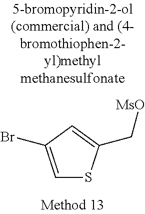 [SMILES: CS(=O)(=O)OCc1cc(Br)cs1] |
| 1AX | 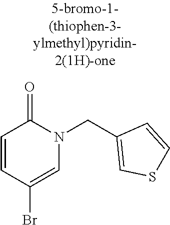 [SMILES: O=c1ccc(Br)cn1Cc1ccsc1] | | 271.7 | 5-bromopyridin-2-ol (commercial) and 3- bromomethylthiophene (commercial) |
| 1AY | 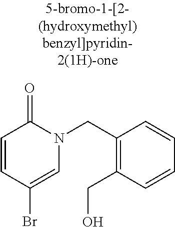 [SMILES: O=c1ccc(Br)cn1Cc1ccccc1CO] | | 295.7 | 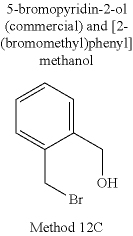 |
| 1AZ | 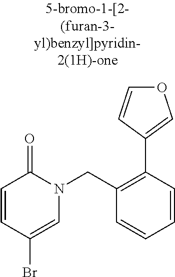 [SMILES: O=c1ccc(Br)cn1Cc1ccccc1-c1ccoc1] | | 331.9 | 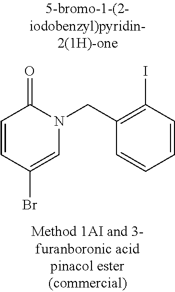 [SMILES: O=c1ccc(Br)cn1Cc1ccccc1I] |
| 1BA | 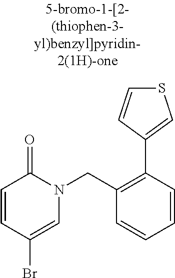 [SMILES: O=c1ccc(Br)cn1Cc1ccccc1-c1ccsc1] | | 347.7 | 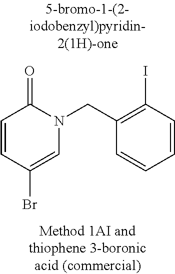 [SMILES: O=c1ccc(Br)cn1Cc1ccccc1I] |
| 1BB | 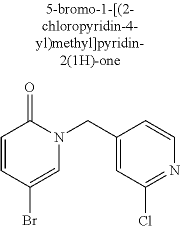 [SMILES: O=c1ccc(Br)cn1Cc1ccnc(Cl)c1] | | 300.7 | 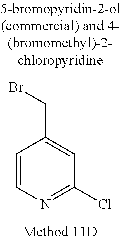 |
| 1BC | 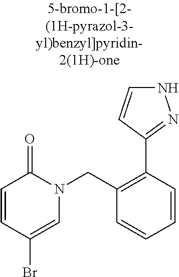 [SMILES: O=c1ccc(Br)cn1Cc1ccccc1-c1cc[nH]n1] | | 331.0 | 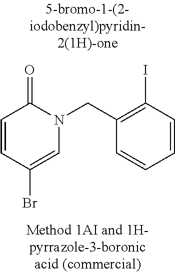 [SMILES: O=c1ccc(Br)cn1Cc1ccccc1I] |
| 1BD | 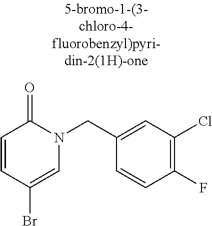 [SMILES: O=c1ccc(Br)cn1Cc1ccc(F)c(Cl)c1] | | 317.4 | 5-bromopyridin-2-ol (commercial) and 3- chloro-4-fluorobenzyl bromide (commercial) |
| 1BE | 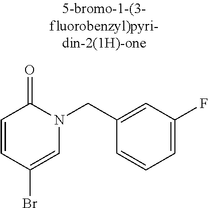 [SMILES: O=c1ccc(Br)cn1Cc1cccc(F)c1] | (400 MHz, DMSO-d6) δ 8.20 (d, J = 2.8 Hz, 1H), 7.56 (dd, J = 9.7, 2.8 Hz, 1H), 7.44- 7.35 (m, 1H), 7.16 (t, J = 7.6 Hz, 3H), 6.43 (d, J = 9.7 Hz, 1H), 5.08 (s, 2H). | 283.8 | 5-bromopyridin-2-ol (commercial) and 3- fluorobenzyl bromide (commercial) |
| 1BF | 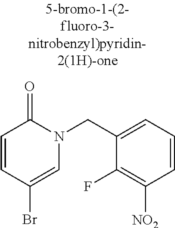 [SMILES: O=c1ccc(Br)cn1Cc1cccc([N+](=O)[O-])c1F] | | 328.7 | 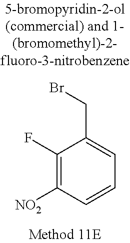 [SMILES: O=[N+]([O-])c1cccc(CBr)c1F] |
| 1BG | 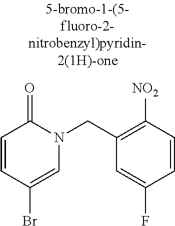 [SMILES: O=c1ccc(Br)cn1Cc1cc(F)ccc1[N+](=O)[O-]] | | | 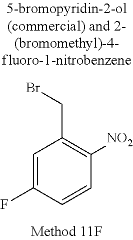 [SMILES: O=[N+]([O-])c1ccc(F)cc1CBr] |
| 1BH | 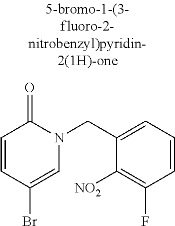 [SMILES: O=c1ccc(Br)cn1Cc1cccc(F)c1[N+](=O)[O-]] | | 328.8 | 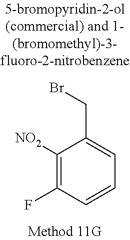 [SMILES: O=[N+]([O-])c1c(F)cccc1CBr] |
| 1BI | 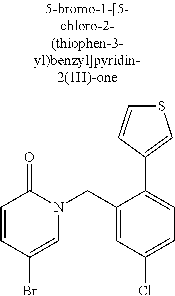 [SMILES: O=c1ccc(Br)cn1Cc1cc(Cl)ccc1-c1ccsc1] | | 381.6 | 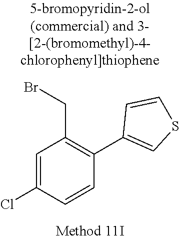 [SMILES: Clc1ccc(-c2ccsc2)c(CBr)c1] |
| 1BJ | 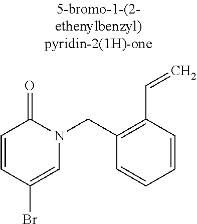 [SMILES: C=Cc1ccccc1Cn1cc(Br)ccc1=O] | | 291.6 | 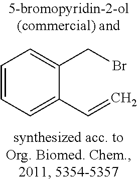 |
| 1BL | 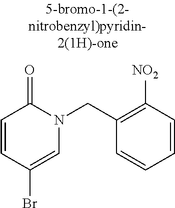 [SMILES: O=c1ccc(Br)cn1Cc1ccccc1[N+](=O)[O-]] | | 310.9 | 5-bromopyridin-2-ol (commercial) and 2- nitrobenzyl bromide (commercial) |
| 1BO | 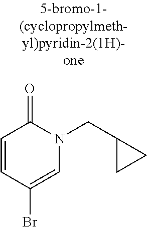 [SMILES: O=c1ccc(Br)cn1CC1CC1] | (400 MHz, DMSO-d6) δ 8.05 (d, J = 2.8 Hz, 1H), 7.52 (dd, J = 9.7, 2.8 Hz, 1H), 6.38 (d, J = 9.7 Hz, 1H), 3.72 (d, J = 7.2 Hz, 2H), 1.29- 1.14 (m, 1H), 0.51-0.43 (m, 2H), 0.42- 0.35 (m, 2H). | 229.9 | 5-bromopyridin-2-ol (commercial) and cyclopropylmethyl- bromide (commercial) |
| 1BP | 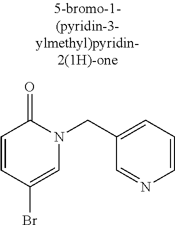 [SMILES: O=c1ccc(Br)cn1Cc1cccnc1] | | | 5-bromopyridin-2-ol (commercial) and 3- (bromomethyl)pyridine hydrobromide (commercial) |
| 1BS | 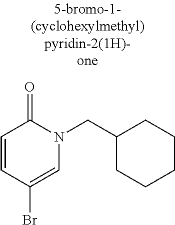 [SMILES: O=c1ccc(Br)cn1CC1CCCCC1] | (400 MHz, DMSO-d6) δ 7.95 (d, J = 2.8 Hz, 1H), 7.51 (dd, J = 9.7, 2.8 Hz, 1H), 6.36 (d, J = 9.7 Hz, 1H), 3.71 (d, J = 7.3 Hz, 2H), 1.75- 1.47 (m, 6H), 1.23-1.09 (m, 3H), 1.06- 0.88 (m, 2H). | 271.9 | 5-bromopyridin-2-ol (commercial) and cyclohexylmethyl bromide (commercial) |
| 1BU | 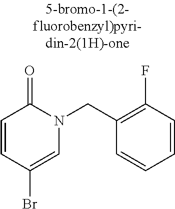 [SMILES: O=c1ccc(Br)cn1Cc1ccccc1F] | (400 MHz, DMSO-d6) δ 8.31 (s, 1H), 7.90 (s, 1H), 5.02 (s, 1H), 4.37 (s, 1H), 2.59 (s, 3H), 2.33 (dd, J = 12.8, 3.4 Hz, 2H), 2.03 (s, 5H), 1.84 (d, J = 12.1 Hz, 2H), 1.63 (d, J = 12.4 Hz, 2H). | 283.0 | 5-bromopyridin-2-ol (commercial) and 2- fluorobenzyl bromide (commercial) |
| 1BW | 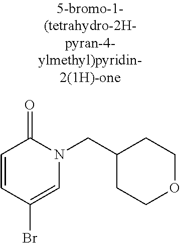 [SMILES: O=c1ccc(Br)cn1CC1CCOCC1] | (400 MHz, DMSO) δ 7.96 (dd, J = 2.8, 0.5 Hz, 1H), 7.63 (dd, J = 9.8, 2.8 Hz, 1H), 7.56-7.39 (m, 5H), 6.48 (dd, J = 9.8, 0.6 Hz, 1H) | 273.9 | 5-bromopyridin-2-ol (commercial) and 4- (bromomethyl)tetra- hydropyran (commercial) |
| 1CA | 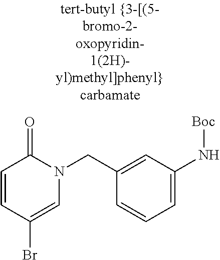 [SMILES: CC(C)(C)OC(=O)Nc1cccc(Cn2cc(Br)ccc2=O)c1] | | 380.7 | 5-bromopyridin-2-ol (commercial) and tert- butyl 3- (bromomethyl)phenyl carbamate (commercial) |
| 1CB | 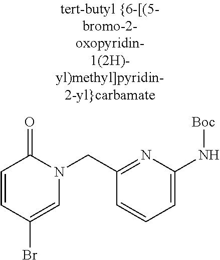 [SMILES: CC(C)(C)OC(=O)Nc1cccc(Cn2cc(Br)ccc2=O)n1] | | | 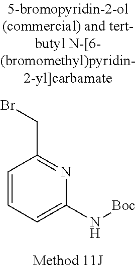 [SMILES: CC(C)(C)OC(=O)Nc1cccc(CBr)n1] |
| 1CC | 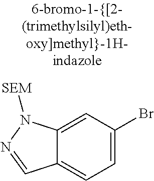 [SMILES: C[Si](C)(C)CCOCn1ncc2ccc(Br)cc21] | | | 6-bromo-1H-indazole (commercial) and 2- (trimethylsilyl) ethoxymethyl chloride (commercial) |
| 1CD | 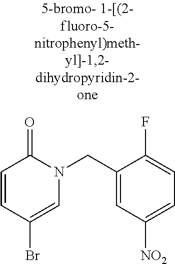 [SMILES: O=c1ccc(Br)cn1Cc1cc([N+](=O)[O-])ccc1F] | | | 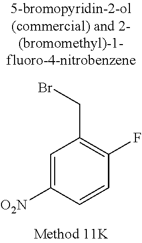 [SMILES: O=[N+]([O-])c1ccc(F)c(CBr)c1] |
| 1CE | 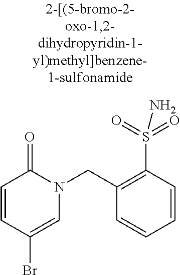 [SMILES: NS(=O)(=O)c1ccccc1Cn1cc(Br)ccc1=O] | | | 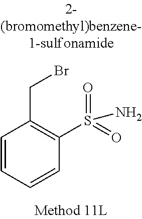 [SMILES: NS(=O)(=O)c1ccccc1CBr] |

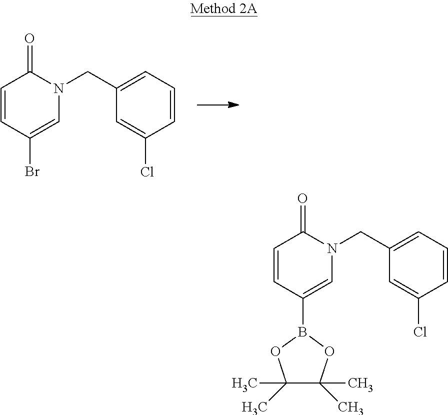 [SMILES: O=c1ccc(Br)cn1Cc1cccc(Cl)c1 ; CC1(C)OB(c2ccc(=O)n(Cc3cccc(Cl)c3)c2)OC1(C)C]

### 1-[(3-chlorophenyl)methyl]-5-(tetramethyl-1,3,2-dioxaborolan-2-yl)-1,2-dihydropyridin-2-one

A 5-bromo-1-(3-chlorobenzyl)pyridin-2(1H)-one (Method 1N) (6.7 g, 22.3 mmol), bis(pinacolato)diboron (5.7 g, 22.3 mmol), potassium acetate (5.9 g, 67.0 mmol), X-Phos (1.6 g, 3.3 mmol) in dry 1,4-dioxane (50 mL) were placed in a sealed tube under argon purge. The reaction mixture was degassed for a further 10 min under a slow stream of argon, at which point palladium(II) acetate (0.5 g) was added. The reaction mixture was heated at 80° C. for 30 min. After cooled to ambient temperature reaction mixture was filtered through Celite, washed with ethyl acetate and solvents were evaporated under reduced pressure. Crude product was purified by flash chromatography (silica gel; hexane/ethyl acetate 1:1) to give 1-[(3-chlorophenyl)methyl]-5-(tetramethyl-1,3,2-dioxaborolan-2-yl)-1,2-dihydropyridin-2-one as a dark yellow oil (6.3 g); yield 82%. LC-MS (m/z) 345.9 (M+1).

The following examples were prepared by the procedure of Method 2A, using the appropriate starting materials.

| Method | Product | m/z | Starting material |
|---|---|---|---|
| 2B | 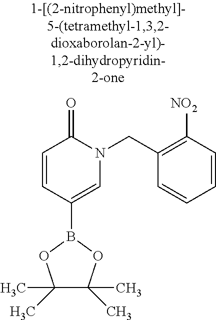 [SMILES: CC1(C)OB(c2ccc(=O)n(Cc3ccccc3[N+](=O)[O-])c2)OC1(C)C] | 357.0 | 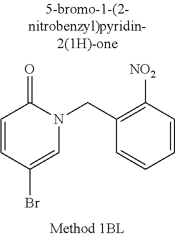 [SMILES: O=c1ccc(Br)cn1Cc1ccccc1[N+](=O)[O-]] |
| 2E | 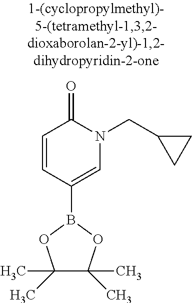 [SMILES: CC1(C)OB(c2ccc(=O)n(CC3CC3)c2)OC1(C)C] | 276.0 | 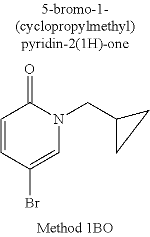 [SMILES: O=c1ccc(Br)cn1CC1CC1] |
| 2F | 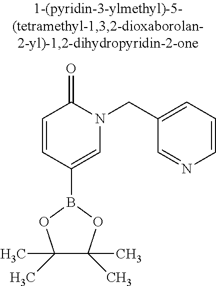 [SMILES: CC1(C)OB(c2ccc(=O)n(Cc3cccnc3)c2)OC1(C)C] | 313.0 | 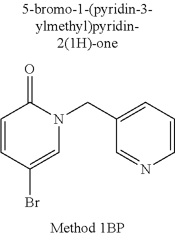 [SMILES: O=c1ccc(Br)cn1Cc1cccnc1] |
| 2H | 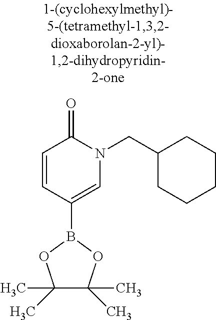 [SMILES: CC1(C)OB(c2ccc(=O)n(CC3CCCCC3)c2)OC1(C)C] | 318.1 | 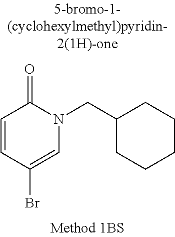 [SMILES: O=c1ccc(Br)cn1CC1CCCCC1] |
| 2K | 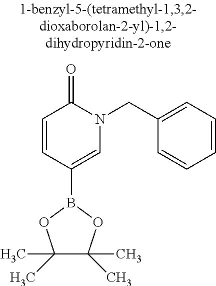 [SMILES: CC1(C)OB(c2ccc(=O)n(Cc3ccccc3)c2)OC1(C)C] | 312.1 | 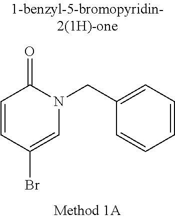 [SMILES: O=c1ccc(Br)cn1Cc1ccccc1] |
| 2L | 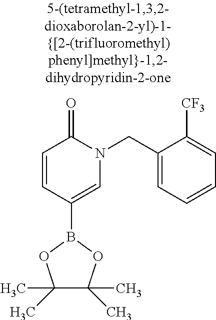 [SMILES: CC1(C)OB(c2ccc(=O)n(Cc3ccccc3C(F)(F)F)c2)OC1(C)C] | | 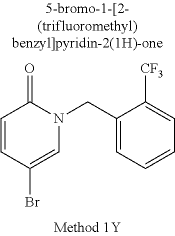 [SMILES: O=c1ccc(Br)cn1Cc1ccccc1C(F)(F)F] |
| 2M | 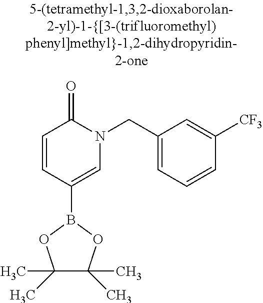 [SMILES: CC1(C)OB(c2ccc(=O)n(Cc3cccc(C(F)(F)F)c3)c2)OC1(C)C] | 380.0 | 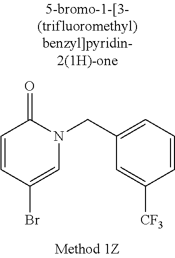 [SMILES: O=c1ccc(Br)cn1Cc1cccc(C(F)(F)F)c1] |
| 2N | 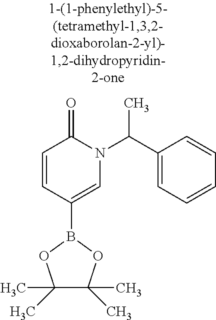 [SMILES: CC(c1ccccc1)n1cc(B2OC(C)(C)C(C)(C)O2)ccc1=O] | 326.1 | 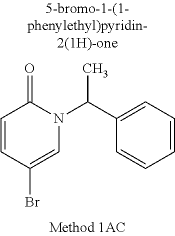 [SMILES: CC(c1ccccc1)n1cc(Br)ccc1=O] |
| 2O | 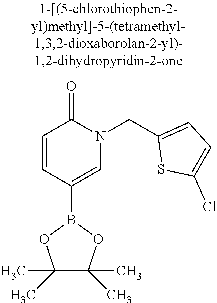 [SMILES: CC1(C)OB(c2ccc(=O)n(Cc3ccc(Cl)s3)c2)OC1(C)C] | 352.0 | 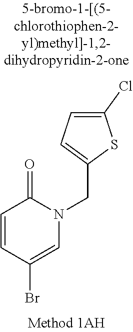 [SMILES: O=c1ccc(Br)cn1Cc1ccc(Cl)s1] |
| 2P | 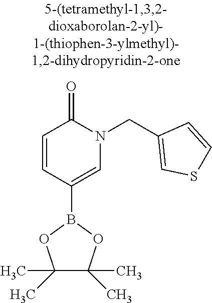 [SMILES: CC1(C)OB(c2ccc(=O)n(Cc3ccsc3)c2)OC1(C)C] | 318.0 | 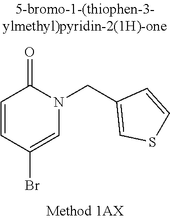 [SMILES: O=c1ccc(Br)cn1Cc1ccsc1] |
| 2R | 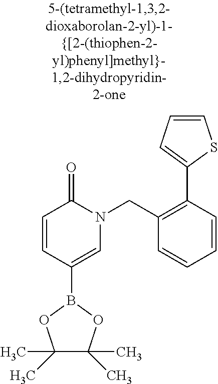 [SMILES: CC1(C)OB(c2ccc(=O)n(Cc3ccccc3-c3cccs3)c2)OC1(C)C] | 394.0 | 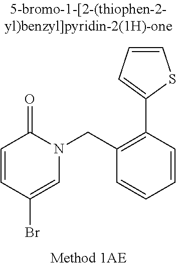 [SMILES: O=c1ccc(Br)cn1Cc1ccccc1-c1cccs1] |
| 2S | 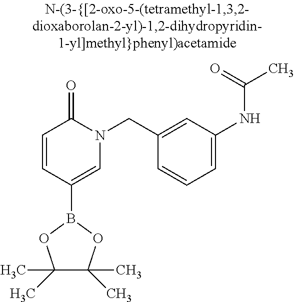 [SMILES: CC(=O)Nc1cccc(Cn2cc(B3OC(C)(C)C(C)(C)O3)ccc2=O)c1] | 368.9 | 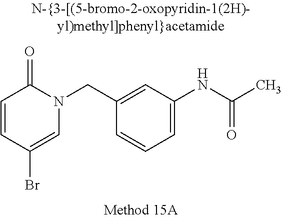 [SMILES: CC(=O)Nc1cccc(Cn2cc(Br)ccc2=O)c1] |
| 2T | 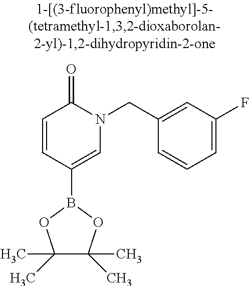 [SMILES: CC1(C)OB(c2ccc(=O)n(Cc3cccc(F)c3)c2)OC1(C)C] | 329.9 | 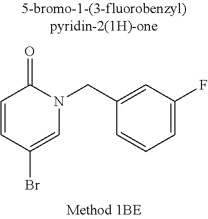 [SMILES: O=c1ccc(Br)cn1Cc1cccc(F)c1] |
| 2U | 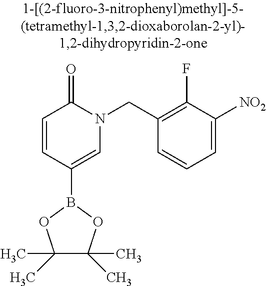 [SMILES: CC1(C)OB(c2ccc(=O)n(Cc3cccc([N+](=O)[O-])c3F)c2)OC1(C)C] | 374.9 | 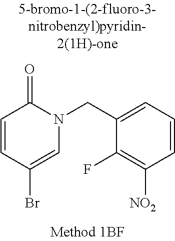 [SMILES: O=c1ccc(Br)cn1Cc1cccc([N+](=O)[O-])c1F] |
| 2W | 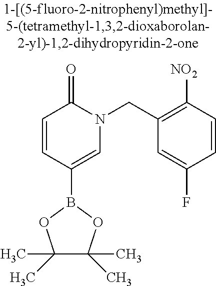 [SMILES: CC1(C)OB(c2ccc(=O)n(Cc3cc(F)ccc3[N+](=O)[O-])c2)OC1(C)C] | 374.9 | 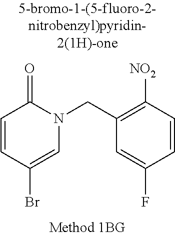 [SMILES: O=c1ccc(Br)cn1Cc1cc(F)ccc1[N+](=O)[O-]] |
| 2X | 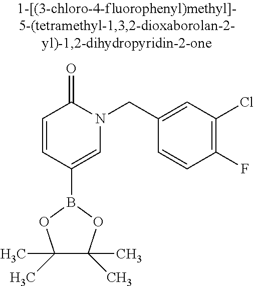 [SMILES: CC1(C)OB(c2ccc(=O)n(Cc3ccc(F)c(Cl)c3)c2)OC1(C)C] | 363.9 | 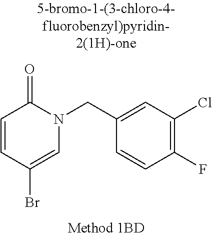 [SMILES: O=c1ccc(Br)cn1Cc1ccc(F)c(Cl)c1] |
| 2Y | 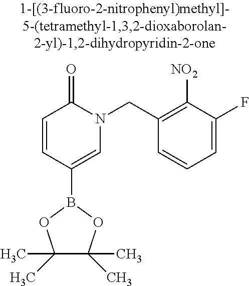 [SMILES: CC1(C)OB(c2ccc(=O)n(Cc3cccc(F)c3[N+](=O)[O-])c2)OC1(C)C] | 374.9 | 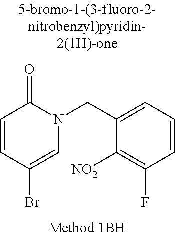 [SMILES: O=c1ccc(Br)cn1Cc1cccc(F)c1[N+](=O)[O-]] |
| 2Z | 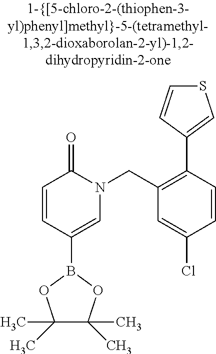 [SMILES: CC1(C)OB(c2ccc(=O)n(Cc3cc(Cl)ccc3-c3ccsc3)c2)OC1(C)C] | 427.9 | 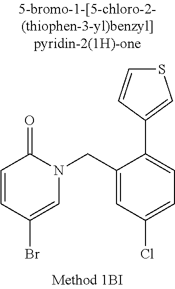 [SMILES: O=c1ccc(Br)cn1Cc1cc(Cl)ccc1-c1ccsc1] |
| 2AA | 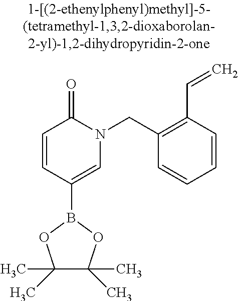 [SMILES: C=Cc1ccccc1Cn1cc(B2OC(C)(C)C(C)(C)O2)ccc1=O] | 337.8 | 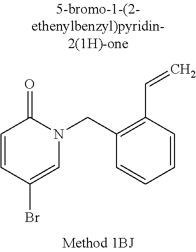 [SMILES: C=Cc1ccccc1Cn1cc(Br)ccc1=O] |
| 2AB | 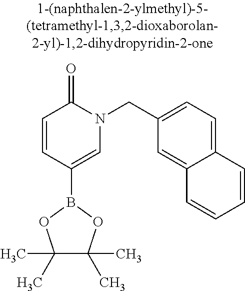 [SMILES: CC1(C)OB(c2ccc(=O)n(Cc3ccc4ccccc4c3)c2)OC1(C)C] | 362.0 | 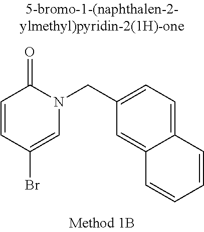 [SMILES: O=c1ccc(Br)cn1Cc1ccc2ccccc2c1] |
| 2AD | 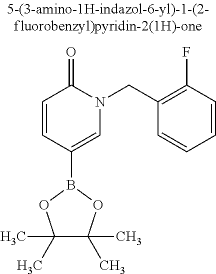 [SMILES: CC1(C)OB(c2ccc(=O)n(Cc3ccccc3F)c2)OC1(C)C] | 329.8 | 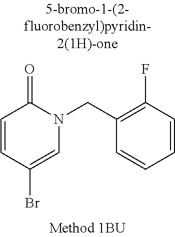 [SMILES: O=c1ccc(Br)cn1Cc1ccccc1F] |
| 2AE | 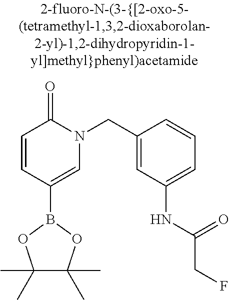 [SMILES: CC1(C)OB(c2ccc(=O)n(Cc3cccc(NC(=O)CF)c3)c2)OC1(C)C] | | 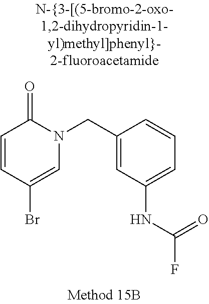 [SMILES: O=C(F)Nc1cccc(Cn2cc(Br)ccc2=O)c1] |
| 2AF | 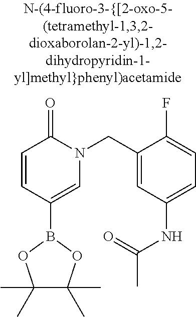 [SMILES: CC(=O)Nc1ccc(F)c(Cn2cc(B3OC(C)(C)C(C)(C)O3)ccc2=O)c1] | | 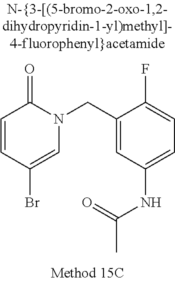 [SMILES: CC(=O)Nc1ccc(F)c(Cn2cc(Br)ccc2=O)c1] |
| 2AG | 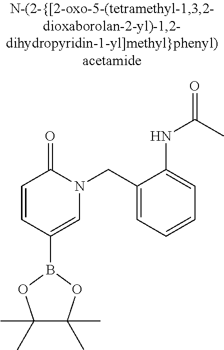 [SMILES: CC(=O)Nc1ccccc1Cn1cc(B2OC(C)(C)C(C)(C)O2)ccc1=O] | | 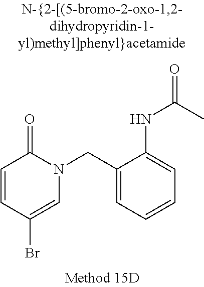 [SMILES: CC(=O)Nc1ccccc1Cn1cc(Br)ccc1=O] |
| 2AH | 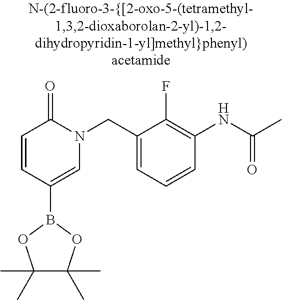 [SMILES: CC(=O)Nc1cccc(Cn2cc(B3OC(C)(C)C(C)(C)O3)ccc2=O)c1F] | | 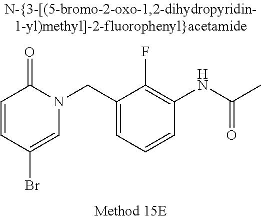 [SMILES: CC(=O)Nc1cccc(Cn2cc(Br)ccc2=O)c1F] |
| 2AI | 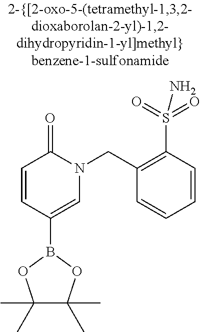 [SMILES: CC1(C)OB(c2ccc(=O)n(Cc3ccccc3S(N)(=O)=O)c2)OC1(C)C] | | 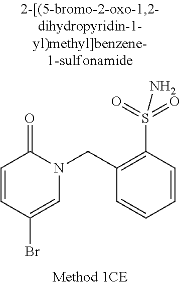 [SMILES: NS(=O)(=O)c1ccccc1Cn1cc(Br)ccc1=O] |

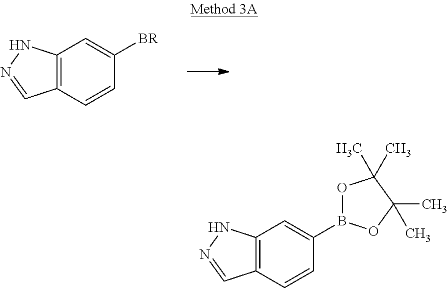

### 6-(tetramethyl-1,3,2-dioxaborolan-2-yl)-1H-indazole

A 6-bromo-1H-indazole (3.0 g, 15.2 mmol), bis(pinacolato)diboron (7.7 g, 30.4 mmol), potassium acetate (5.9 g, 60.9 mmol) in dry N,N-dimethylformamide (40 mL) were placed in a sealed tube under argon purge. The reaction mixture was degassed for a further 10 min with a slow stream of argon, at which point [1,1′-bis(diphenylphosphino)ferrocene]palladium(II) chloride, complex with dichloromethane (0.3 g) was added. The reaction mixture was heated at 100° C. overnight. After cooled to ambient temperature reaction mixture was filtered through Celite, washed with ethyl acetate and solvents were evaporated under reduced pressure. Crude product was purified by flash chromatography (silica gel; hexane/ethyl acetate 1:1) to give 6-(tetramethyl-1,3,2-dioxaborolan-2-yl)-1H-indazole (3.0 g); yield 81%. LC-MS (m/z) 244.9 (M+1).

The following examples were prepared by the procedure of Method 3A, using the appropriate starting materials.

| Meth- | | | |
|---|---|---|---|
| od | Product | m/z | Starting materials |
| 3B | 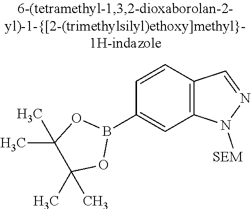 [SMILES: CC1(C)OB(c2ccc3cnn(COCC[Si](C)(C)C)c3c2)OC1(C)C] | 375.0 | 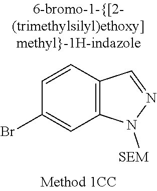 [SMILES: C[Si](C)(C)CCOCn1ncc2ccc(Br)cc21] |

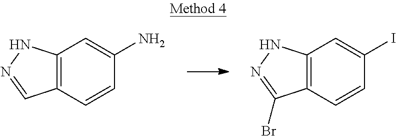 [SMILES: Nc1ccc2cn[nH]c2c1 ; Brc1n[nH]c2cc(I)ccc12]

### 3-bromo-6-iodo-1H-indazole

6-Aminoindazole (1.0 g, 7.5 mmol) was mixed with ice (6 g) and water (3.5 mL). The reaction mixture was cooled to 0° C. and concentrated aqueous hydrochloride solution (3.8 mL) was added followed by a solution of sodium nitrite (0.6 g, 8.2 mmol) in water (2.5 mL). After 10 min of stirring at 0° C. potassium iodide (1.3 g, 9.0 mmol) was added in few portions. Then the cold bath was removed and reaction mixture was warmed to 40° C., heated for 40 min and next the temperature was increased to 50° C. and heated for another 30 min. After cooled to ambient temperature the solution was alkalized with 10% NaOH. The brown precipitate was collected by filtration and washed with saturated aqueous solution of sodium hydrogen carbonate. The crude product was dissolved in tetrahydrofuran (25 mL) and refluxed with silica gel for 10 min. To this slurry hexane was added and the mixture was vaccum filtered through a silica pad. The silica was washed with solution of tetrahydrofuran in hexane (2:3). The filtrate was concentrated under reduced pressure to give 6-iodoindazole (0.9 g). The 6-iodoindazole (0.9 g, 3.9 mmol) was dissolved in dry dichloromethane (30 mL), cooled to 0° C. and N-bromosuccinimide (0.8 g, 4.3 mmol) was added in portions. The reaction mixture was stirred at 0° C. for 1 h. The precipitate was collected by filtration and washed with dichloromethane. The obtained product 3-bromo-6-iodo-1H-indazole was used to the next step without further purification. LC-MS (m/z) 324.8 (M+1).

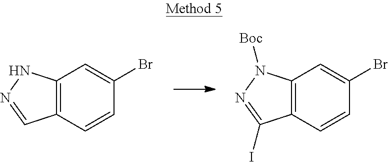 [SMILES: Brc1ccc2cn[nH]c2c1 ; CC(C)(C)OC(=O)n1nc(I)c2ccc(Br)cc21]

### tert-butyl 6-bromo-3-iodo-1H-indazole-1-carboxylate

To a solution of 6-bromo-1H-indazole (0.7 g, 3.7 mmol) in dry N,N-dimethylformamide (10 mL) potassium hydroxide (0.5 g, 9.3 mmol) was added. The reaction mixture was cooled to 0° C. and then iodine (1.4 g, 5.6 mmol) dissolved in dry N,N-dimethylformamide (5 mL) was dropped. The stirring was continuous for next 18 h at room temperature. Water was added and the mixture was extracted with ethyl acetate. Combined organic phase were washed with saturated solution of sodium sulfite, dried over sodium sulfate and concentrated under reduced pressure. Then to the solution of crude product (1.1 g, 3.5 mmol), triethylamine (0.9 mL, 7.0 mmol) in dry dichloromethane (20 mL), cooled to 0° C. di-tert-butyl dicarbonate (0.8 g, 3.5 mmol) dissolved in dry dichloromethane (5 mL) was dropped. The stirring was continuous overnight at room temperature. Next the solution was washed with water then brine, dried over anhydrous sodium sulfate and concentrated under reduced pressure. Obtained product (1.45 g) was used to the next step without further purification. LC-MS (m/z) 424.8 (M+1).

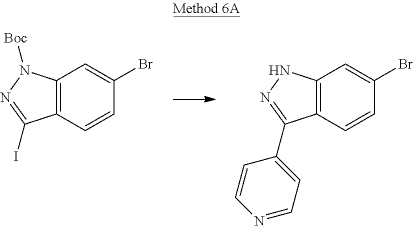 [SMILES: CC(C)(C)OC(=O)n1nc(I)c2ccc(Br)cc21 ; Brc1ccc2c(-c3ccncc3)n[nH]c2c1]

### 6-bromo-3-(pyridin-4-yl)-1H-indazole

A tert-butyl 6-bromo-3-iodo-1H-indazole-1-carboxylate (Method 5) (0.2 g, 0.47 mmol), pyridine 4-boronic acid (0.11 g, 0.9 mmol), caesium carbonate (0.41 g, 1.3 mmol) in mixture dioxane/water (2:1) (3 mL) were placed in a sealed tube under argon purge. The reaction mixture was degassed for a further 10 min under a slow stream of argon, at which point [1,1′-bis(diphenylphosphino)ferrocene]palladium(II) chloride, complex with dichloromethane (10 mg) was added. The reaction mixture was heated at 120° C. under microwave irradiation for 20 min. After cooled to ambient temperature reaction mixture was filtered through Celite, washed with ethyl acetate and solvents were evaporated under reduced pressure. Crude product was purified by flash chromatography (hexane/ethyl acetate 1:1) to give 6-bromo-3-(pyridin-4-yl)-1H-indazole (0.12 g); yield 67%; LC-MS (m/z) 275.1 (M+1).

The following examples were prepared by the procedure of Method 6A, using the appropriate starting materials.

| Meth- | | | |
|---|---|---|---|
| od | Product | m/z | Starting materials |
| 6B | 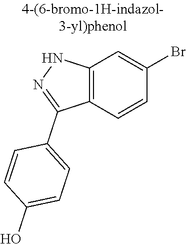 [SMILES: Oc1ccc(-c2n[nH]c3cc(Br)ccc23)cc1] | 290.8 | 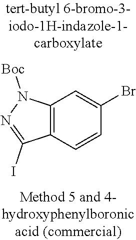 [SMILES: CC(C)(C)OC(=O)n1nc(I)c2ccc(Br)cc21] |
| 6C | 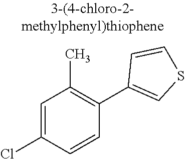 | | 2-bromo-5- chlorotoluene (commercial) and 3-thiopheneboronic acid (commercial) |

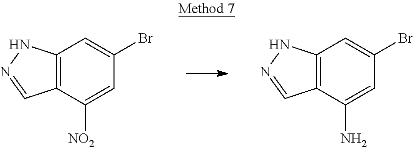 [SMILES: O=[N+]([O-])c1cc(Br)cc2[nH]ncc12 ; Nc1cc(Br)cc2[nH]ncc12]

### 4-amino-6-bromoindazole

A 4-nitro-6-bromoindazole (0.2 g, 0.8 mmol), iron (0.23 g, 4.1 mmol) were suspended in a mixture of methanol (4 mL) and acetic acid (1 mL) and heated at reflux for 1.5 hour. After cooled to the ambient temperature solvents were evaporated under reduced pressure. The crude product was dissolved in ethyl acetate and washed with 1M aqueous solution of sodium hydroxide, dried over sodium sulfate and concentrated under reduced pressure. Obtained product was used to the next step without further purification. LC-MS (m/z) 213.9 (M+1).

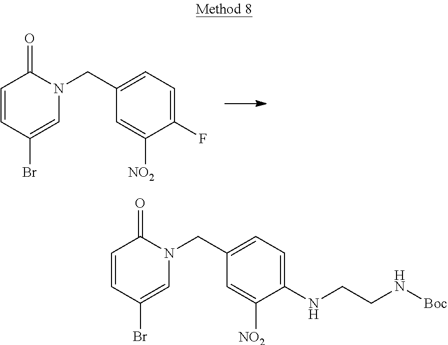

### tert-butyl N-[2-({4-[(5-bromo-2-oxo-1,2-dihydropyridin-1-yl)methyl]-2-nitrophenyl}amino)ethyl]carbamate

A mixture of 5-bromo-1-(4-fluoro-3-nitrobenzyl)pyridin-2(1H)-one (Method 1K) (0.15 g, 0.5 mmol), tert-butyl 2-aminoethylcarbamate (0.15 g, 0.9 mmol) and diisopropylenediamine (0.12 mL, 0.7 mmol) in dry tetrahydrofuran (10 mL) was stirred at room temperature overnight. The reaction mixture was concentrated under reduced pressure to dryness. The crude residue was partitioned between water and dichloromethane and the organic layer was washed with water, dried over anhydrous sodium sulfate, filtered, and concentrated under reduced pressure. Obtained product was used to the next step without further purification (0.18 g); yield 84%. LC-MS (m/z) 505.1 (M+1).

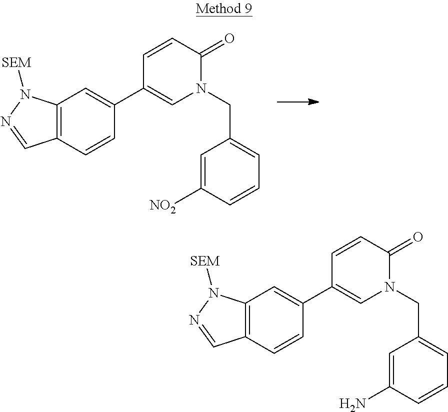 [SMILES: C[Si](C)(C)CCOCn1ncc2ccc(-c3ccc(=O)n(Cc4cccc([N+](=O)[O-])c4)c3)cc21 ; C[Si](C)(C)CCOCn1ncc2ccc(-c3ccc(=O)n(Cc4cccc(N)c4)c3)cc21]

### 1-[(3-aminophenyl)methyl]-5-(1-{[2-(trimethylsilyl)ethoxy]methyl}-1H-indazol-6-yl)-1,2-dihydropyridin-2-one

To a solution of 1-[(3-nitrophenyl)methyl]-5-(1-{[2-(trimethylsilyl)ethoxy]methyl}-1H-indazol-6-yl)-1,2-dihydropyridin-2-one (Example 1BP) (0.2 g, 0.4 mmol) and saturated solution of copper (II) acetate (1.5 mL) in methanol (10 mL), cooled to 0° C., sodium borohydride (0.3 g, 8 mmol) was added in portions. After 30 min ice bath was removed and the reaction mixture was stirred for 2 h at room temperature. Then methanol was concentrated under reduced pressure and the residue was partitioned between water and dichloromethane. The water phase was extracted with dichloromethane. Combined organic phases were washed with brine, dried over anhydrous sodium sulfate, filtered, and concentrated under reduced pressure. The crude product was purified by flash chromatography (ethyl acetate/methanol 9:1) to give 1-[(3-aminophenyl)methyl]-5-(1-{[2-(trimethylsilyl)ethoxy]methyl}-1H-indazol-6-yl)-1,2-dihydropyridin-2-one as an oil (0.15 g); yield 80%. LC-MS (m/z) 447.2 (M+1).

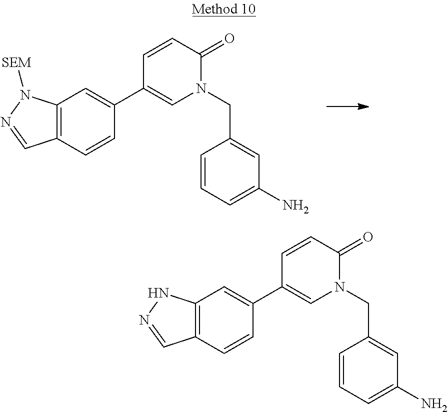

### 1-[(3-aminophenyl)methyl]-5-(1H-indazol-6-yl)-1,2-dihydropyridin-2-one

To the solution of 1-[(3-aminophenyl)methyl]-5-(1-{[2-(trimethylsilyl)ethoxy]methyl}-1H-indazol-6-yl)-1,2-dihydropyridin-2-one (Method 9) (0.07 g, 0.1 mmol) in tetrahydrofuran (2.5 mL) 1.8M solution of tetra-n-butylammonium fluoride in tetrahydrofuran (2.5 mL) and 3A molecular sieves were added. The reaction mixture was stirred at 70° C. for 4 h. The molecular sieves were filtered off and filtrate was evaporated under reduced pressure to dryness. The residue was partitioned between water and ethyl acetate. Organic layer was washed with water, then brine, dried over anhydrous sodium sulfate, filtered, and concentrated under reduced pressure. The crude product was used to the next step without further purification. LC-MS (m/z) 364.8 (M+1).

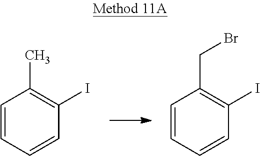

### 1-(bromomethyl)-2-iodobenzene

A mixture of 1-iodo-2-methylbenzene (0.5 g, 2.3 mmol), N-bromosuccinimide (0.7 g, 3.7 mmol) and 2,2′-azobisisobutyronitrile (0.02 g, 0.1 mmol) in tetrachloromethane (10 mL) was heated at reflux until the reaction was completed (tlc control). After cooled down to the ambient temperature the reaction mixture was quenched with water and water layer was extracted with dichloromethane. Combined organic phases were washed with sodium hydrocarbonate, dried over anhydrous sodium sulfate, filtered and concentrated under reduced pressure. The crude product was purified by column chromatography (silica gel; hexane/ethyl acetate 9:1) to give 1-(bromomethyl)-2-iodobenzene (0.5 g) as yellowish oil; yield 69%.

The following examples were prepared by the procedure of Method 11A, using the appropriate starting materials.

| Method | Product | m/z | Starting material |
|---|---|---|---|
| 11B | 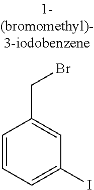 | | 3-iodotoluene (commercial) |
| 11C | 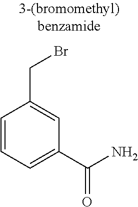 [SMILES: NC(=O)c1cccc(CBr)c1] | 214.0 | m-toluamide (commercial) |
| 11D | 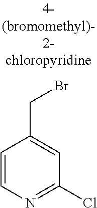 | 207.7 | 2-chloro-4- methylpyridine (commercial) |
| 11E | 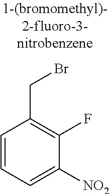 [SMILES: O=[N+]([O-])c1cccc(CBr)c1F] | | 2-fluoro-3- nitrotoluene (commercial) |
| 11F | 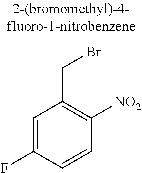 [SMILES: O=[N+]([O-])c1ccc(F)cc1CBr] | | 5-fluoro-2- nitrotoluene (commercial) |
| 11G | 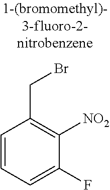 [SMILES: O=[N+]([O-])c1c(F)cccc1CBr] | | 3-fluoro-2- nitrotoluene (commercial) |
| 11H | 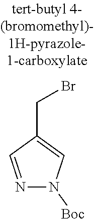 [SMILES: CC(C)(C)OC(=O)n1cc(CBr)cn1] | 262.7 | 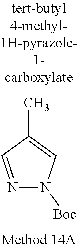 |
| 11I | 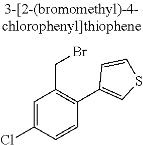 [SMILES: Clc1ccc(-c2ccsc2)c(CBr)c1] | | 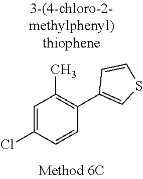 |
| 11J | 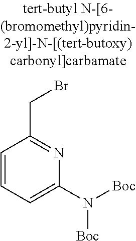 [SMILES: CC(C)(C)OC(=O)N(C(=O)OC(C)(C)C)c1cccc(CBr)n1] | | 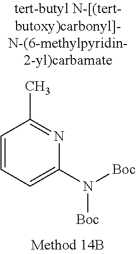 [SMILES: Cc1cccc(N(C(=O)OC(C)(C)C)C(=O)OC(C)(C)C)n1] |
| 11K | 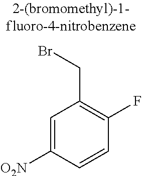 [SMILES: O=[N+]([O-])c1ccc(F)c(CBr)c1] | | 2-fluoro-5- nitrotoluene (commercial) |
| 11L | 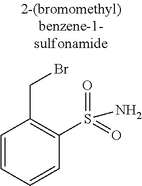 [SMILES: NS(=O)(=O)c1ccccc1CBr] | | 2- methylbenzenesulfonamide |

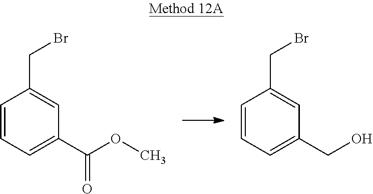

### 3-bromomethylbenzyl alcohol

To a solution of methyl 3-(bromomethyl)benzoate (0.5 g, 2.2 mmol) dissolved in dry toluene (10 mL), cooled to −30° C., 1M solution of DIBAL-H in toluene (4.3 mL, 4.4 mmol) was dropped. The reaction mixture was stirred at −30° C. for 2.5 h and then allowed to warm to ambient temperature. The reaction mixture was quenched with methanol, filtered, extracted with ethyl acetate, washed with aqueous sodium bicarbonate solution, then brine, dried over sodium sulfate, and evaporated under reduced pressure giving 3-bromomethylbenzyl alcohol (0.3 g); yield 68%.

The following examples were prepared by the procedure of Method 12A, using the appropriate starting materials.

| Method | Product | m/z | Starting material |
|---|---|---|---|
| 12C | 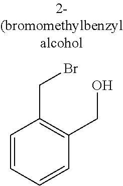 | | 2- (bromomethyl) benzoate (commercial) |

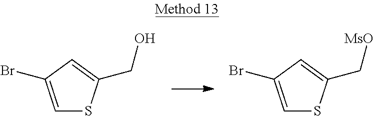 [SMILES: OCc1cc(Br)cs1 ; CS(=O)(=O)OCc1cc(Br)cs1]

### (4-bromothiophen-2-yl)methyl methanesulfonate

To a cooled to 0° C. solution of (4-bromothiophen-2-yl)methanol (1 g, 5.2 mmol) and triethylamine (2.3 mL, 16.6 mmol) in dichloromethane (25 mL) methanesulfonyl chloride (0.6 mL, 7.8 mmol) was added dropwise. When addition was complete, the cooling bath was removed and the reaction was stirred at room temperature until starting material was consumed (tlc control). The reaction mixture was washed with water and the organic layer was dried over sodium sulfate, filtered and concentrated under reduced pressure. The residue was purified by flash chromatography (hexane/ethyl acetate 1:1) to give (4-bromothiophen-2-yl)methyl methanesulfonate (0.4 g); yield 30%.

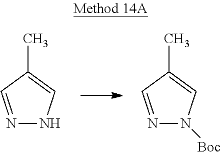

### 4-methyl-pyrazole-1-carboxylic acid tert-butyl ester

To a solution of 4-methyl-H-pyrazole (1 g, 12 mmol) and 4-dimethylaminopyridine (0.15 g, 1.2 mmol) in acetonitrile (20 mL) di-tert-butyl dicarbonate (2.8 g, 13 mmol) was added. The reaction mixture was stirred overnight at room temperature and then concentrated under reduced pressure. The residue was dissolved in ethyl acetate and washed with water, brine, dried over magnesium sulfate, filtered and concentrated under reduced pressure. The crude product was purified by column chromatography (silica gel; hexane/ethyl acetate 4:1) to provide 4-methyl-pyrazole-1-carboxylic acid tert-butyl ester as a light yellow oil (1.8 g); yield 84%.

The following compounds were prepared by the procedure of Method 14A, using the appropriate starting materials.

| Method | Product | Starting material |
|---|---|---|
| 14B | 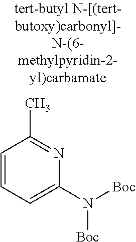 [SMILES: Cc1cccc(N(C(=O)OC(C)(C)C)C(=O)OC(C)(C)C)n1] | 2-amino- 6-picoline (commercial) |

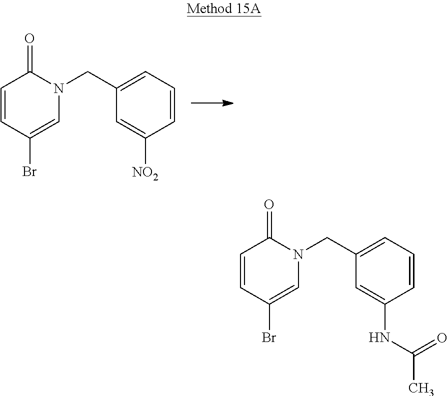 [SMILES: O=c1ccc(Br)cn1Cc1cccc([N+](=O)[O-])c1 ; CC(=O)Nc1cccc(Cn2cc(Br)ccc2=O)c1]

### N-{3-[(5-bromo-2-oxopyridin-1(2H)-yl)methyl]phenyl}acetamide

A mixture of 5-bromo-1-(3-nitrobenzyl)pyridin-2(1H)-one (Example 1J) (0.2 g, 0.6 mmol) and tin (II) chloride (0.7 g, 3.2 mmol) in ethyl acetate were heated at reflux for 30 min. After cooled to the ambient temperature water was added followed by 1M aqueous solution of hydrochloride. Water phase was extracted with ethyl acetate. Combined organic layers were washed with brine, dried over anhydrous sodium sulfate and concentrated under reduced pressure. The crude product was purified by column chromatography (silica gel; dichloromethane/methanol 4:1) to give 1-[(3-aminophenyl)methyl]-5-bromo-1,2-dihydropyridin-2-one (0.18 g). To a solution of obtained product (0.18 g, 0.6 mmol) in dichloromethane (8 mL) pyridine (0.1 g, 1.3 mmol) was added followed by acetyl chloride (0.1 g, 1.3 mmol). The reaction mixture was stirred at room temperature for 3 h. The slurry was evaporated under reduced pressure to dryness. The crude product was purified by column chromatography (silica gel; hexane/ethyl acetate 1:1) to give N-{3-[(5-bromo-2-oxopyridin-1(2H)-yl)methyl]phenyl}acetamide (0.09 g); yield 43%. LC-MS (m/z) 322.7 (M+1).

The following compounds were prepared by the procedure of Method 15A, using the appropriate starting materials.

| Method | Product | Starting material |
|---|---|---|
| 15B | 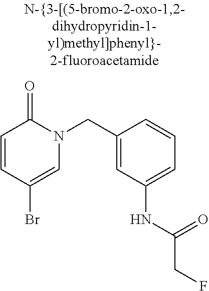 [SMILES: O=C(CF)Nc1cccc(Cn2cc(Br)ccc2=O)c1] | 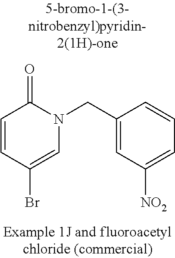 [SMILES: O=c1ccc(Br)cn1Cc1cccc([N+](=O)[O-])c1] |
| 15C | 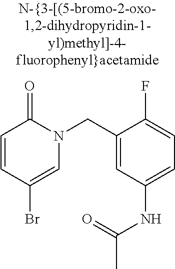 [SMILES: CC(=O)Nc1ccc(F)c(Cn2cc(Br)ccc2=O)c1] | 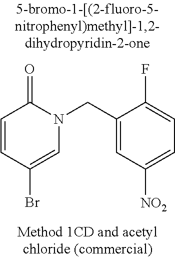 [SMILES: O=c1ccc(Br)cn1Cc1cc([N+](=O)[O-])ccc1F] |
| 15D | 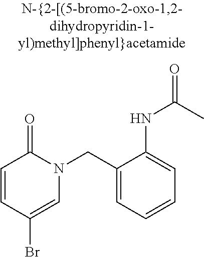 [SMILES: CC(=O)Nc1ccccc1Cn1cc(Br)ccc1=O] | 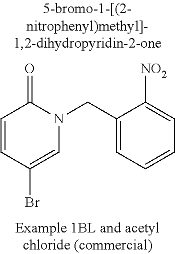 [SMILES: O=c1ccc(Br)cn1Cc1ccccc1[N+](=O)[O-]] |
| 15E | 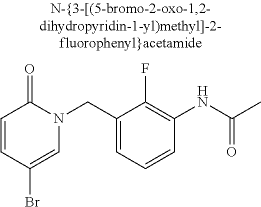 [SMILES: CC(=O)Nc1cccc(Cn2cc(Br)ccc2=O)c1F] | 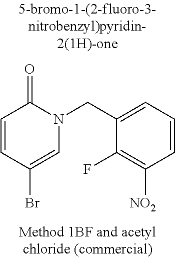 [SMILES: O=c1ccc(Br)cn1Cc1cccc([N+](=O)[O-])c1F] |

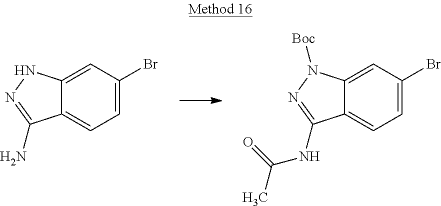 [SMILES: Nc1n[nH]c2cc(Br)ccc12 ; CC(=O)Nc1nn(C(=O)OC(C)(C)C)c2cc(Br)ccc12]

### tert-butyl 6-bromo-3-acetamido-1H-indazole-1-carboxylate

To a solution of 6-bromo-1H-indazol-3-amine (0.5 g, 2.4 mmol) in THF (10 mL) at room temperature was added di-tert-butyl dicarbonate (0.5 g, 2.4 mmol) followed by 4-dimethylaminopyridine (0.01 g, 0.08 mmol). The resulting mixture was allowed to stir for 30 h. Then the reaction mixture was concentrated under reduced pressure to provide a yellowish semisolid. The residue was dissolved in dichloromethane and washed with water. The organic layer was dried over sodium sulfate, filtered and evaporated to dryness under reduced pressure. The crude product was purified by flash chromatography (dichloromethane/methanol 98:2) to give tert-butyl 3-amino-6-bromo-1H-indazole-1-carboxylate (0.7 g, 95%) as a solid. The obtained product (0.1 g, 0.3 mmol) was dissolved in acetic anhydride (2 mL) and 4-dimethylaminopyridine was added. The reaction mixture was stirred at room temperature overnight and then at 100° C. for 3 h. After cooled to ambient temperature the solvent was evaporated under reduced pressure and the crude product was purified by column chromatography (silica gel; dichloromethane 100%). The tert-butyl 6-bromo-3-acetamido-1H-indazole-1-carboxylate was obtained as a solid (0.054 g); yield 61%. LC-MS (m/z) 355.9 (M+1).

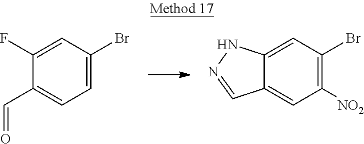 [SMILES: O=Cc1ccc(Br)cc1F ; O=[N+]([O-])c1cc2cn[nH]c2cc1Br]

### 6-bromo-5-nitro-1H-indazole

To a cooled to 0° C. solution of potassium nitrate (0.55 g, 5.4 mmol) in concentrated sulfuric acid (8 mL) 4-bromo-2-fluorobenzaldehyde (1 g, 4.9 mmol) was added. The reaction mixture was stirred at room temperature for 1 h. Then the mixture was poured into ice water and occurred precipitate was collected by filtration. The solid was washed with water next saturated aqueous solution of sodium hydrocarbonate and air dried to give 4-bromo-2-fluoro-5-nitrobenzaldehyde (1.1 g, 90%). To the obtained product (0.1 g, 0.4 mmol) dissolved in ethanol (5 mL) hydrazine monohydrate (0.02 mL, 0.4 mmol) was dropped. The reaction mixture was heated at 80° C. for 8 h. After cooled to room temperature the solution was evaporated under reduced pressure. The residue was dissolved in ethyl acetate and washed with water and next brine. Organic layer was dried over sodium sulfate, filtered and evaporated under reduced pressure to give 6-bromo-5-nitro-1H-indazole as a solid (0.09, 95%). ¹H NMR (300 MHz, DMSO) δ 13.71 (s, 1H), 8.61 (d, J=3.1 Hz, 1H), 8.33 (s, 1H), 8.05 (s, 1H).

### 3.2. Determination of the Inhibitory Activity In Vitro

The compounds of the present invention were tested for their inhibitory activity against MNK1 and MNK2 kinases once or several times. When tested more than once, the data are reported herein as average value, wherein the average value, also referred to as the mean value, represents the sum of obtained values divided by the number of times tested.

MNK-inhibitory activity of the compounds of the present invention was tested using the ADP-Glo assay as described in the following paragraphs. The procedure for determining the % of inhibition and the IC50 values with the ADP-Glo assay in in vitro kinase assays consists of two parts:

1. Kinase reaction performed under optimized conditions;
2. Detection of ADP as a product of the reaction using ADP-Glo™ system (Promega).

The tested compounds indicated in Table 1 below were dissolved in DMSO, then transferred to the V-bottom plate to perform one concentration (% of inhibition) or nine serial dilutions of the compound (in order to obtain IC50 curves) in 25% DMSO, as indicated in Table 1 below.

The optimized conditions for performing MNK1 in vitro kinase assay were as follows:

| | Final concentration/final |
|---|---|
| Reagent/condition | condition |
| Buffer | 60 mM HEPES, pH 7.5 |
| MgCl₂ | 3 mM |
| MnCl₂ | 3 mM |
| PEG_{20.000} | 50 μg/ml |
| DTT | 1 mM |
| ATP (Km) (ultrapure, from ADP-Glo ™ kit) | 21 μM |
| Substrate (Km): | 250 μM |
| GRSRSRSRSR (Lipopharm) | |
| Enzyme - MNK1 (Carna Bioscence) catalog | 22.52 nM |
| no. 02-145 | |
| Time of reaction | 2 h |
| Temperature of reaction | 25° C. |

The optimized conditions for performing MNK2 in vitro kinase assay were as follows:

| | Final concentration/final |
|---|---|
| Reagent/condition | condition |
| Buffer | 5 mM MOPS, pH 7.5 |
| MgCl₂ | 3 mM |
| EDTA | 0.4 mM |
| DTT | 0.25 mM |
| ATP (Km) (ultrapure, from ADP-Glo ™ kit) | 100 μM for % of inhibition |
| | 200 μM for IC50 |
| Substrate (Km): | 160 μM |
| GRSRSRSRSR (Lipopharm) | |
| Enzyme - MNK2 (Carna Bioscence) catalog | 10.55 nM |
| no. 02-146 | |
| Time of reaction | 2 h |
| Temperature of reaction | 25° C. |

For testing the compounds, the following protocol was used. Two mixes were prepared on ice, Mix 1 containing substrate, ATP and reaction buffer and Mix 2 containing reaction buffer (1 times concentrated) and kinase. 15 μL per well of Mix 1 was transferred to wells of a 96-well plate. Next, 2.5 μL of the diluted compound to be tested was added to Mix 1, followed by the addition of 12.5 μL of Mix 2 per well. Total reaction volume was 30 μL per well. The experiment was performed in duplicate on a single plate (% inhibition) or one repetition of two plates (IC50). Additionally, a positive control was carried out on each plate by the adding reference inhibitory compounds staurosporine and cercosporamide (Liu, Hu, Waller, Wang, & Liu, 2012). These two inhibitory compounds gave the expected results, i.e. they inhibited the enzymes, and thus confirmed that the test was suitable to assess inhibition. For each test, three controls were performed: (i) 30 μL of the reaction mixture containing: reaction buffer, ATP, kinase, DMSO (negative control); (ii) 30 μL of the reaction mixture containing reaction buffer, ATP, DMSO (negative control); (iii) 30 μL of the reaction mixture containing reaction buffer, substrate, kinase, ATP, DMSO (positive control). Final concentration of DMSO in the reaction was 2%. The plate was incubated for 120 minutes on a shaker at 25° C. To detect the ADP amount produced during the kinase reaction, the commercially available kit ADP-Glo™ Kinase Assay (Promega, cat. No# V9103) was used. The protocol used for the detection was based on the Technical Bulletin of the ADP-Glo™ Kinase Assay (Promega) and was adapted to 96-well plate containing 30 μL reaction mixture as follows:

30 μL of ADP-Glo™ Reagent was added to each well of a 96 well plate containing μL of reaction mixture to terminate the kinase reaction and deplete the remaining ATP. The plate was incubated for 100 minutes on a shaker at RT. 60 μL of Kinase Detection Solution was added to each well of 96-well plate containing 60 μL of the solution to convert ADP to ATP and to allow the newly synthesized ATP to be measured using a luciferase/luciferin reaction (ratio of kinase reaction volume to ADP Glo™ Reagent volume to Kinase Detection Solution volume was maintained at 1:1:2). The plate was incubated for 40 minutes on a shaker at RT, protected from light. Luminescence was measured in the plate reader Synergy 2 (BioTek), wherein the luminescent signal is proportional to the ADP concentration produced and thus correlates with kinase activity.

After data normalization to the negative control (no kinase added) by its subtraction, percent of inhibition values was obtained according to the following equation:

$$\% inhibition = 100 \% - ( \frac{{Lum}_{Cpd} \times 100 \%}{{Lum}_{PC}} )$$

% inhibition—percent of inhibition
Lum_{Cpd}—value of compound's luminescence (in RLU)
LumPC—value of positive control's luminescence (in RLU)

The IC50-value was determined using the GraphPad Prism 6.0 [log(agonist) vs. normalized response—Variable slope].

Table 1 below shows the inhibition of kinase activity by representative compounds of the present invention at 1 μM and 5 μM, and the IC50 results.

TABLE 1
Inhibition of MNK1 and MNK2 activity by
compounds of the present invention.

| MNK2 | MNK2 | MNK2 | MNK1 | MNK1 | MNK1 | |
|---|---|---|---|---|---|---|
| IC50 | % INH | % INH | IC50 | % INH | % INH | Compound |
| (nM) | (1 μM) | (5 μM) | (nM) | (1 μM) | (5 μM) | (Example) |
| 479.00 | 70.58 | 94.00 | 348.75 | 80.33 | 90.00 | 1A |
| 429.00 | 70.00 | 86.00 | 370.00 | 69.00 | 88.00 | 2B |
| | 46.00 | 66.00 | | 92.00 | 92.00 | 2AK |
| | | 94.00 | | | 83.00 | 2AW |
| | | 66.00 | | | 47.00 | 2AU |
| | | 60.00 | | | 36.00 | 2AY |
| | | 90.00 | | | 71.00 | 2AX |
| 426.00 | 76.00 | 90.00 | 218.00 | 78.00 | 93.00 | 2C |
| | | 44.00 | | | 51.00 | 9A |
| | | 62.00 | | | 67.00 | 2F |
| | 35.00 | 55.00 | | 75.00 | 81.00 | 2G |
| | 46.50 | | | 66.00 | | 1C |
| 541.00 | 85.00 | | 460.00 | 60.00 | | 2I |
| | 49.00 | | | 24.00 | | 2J |
| | 43.00 | | | 43.00 | | 1D |
| 370.00 | 42.00 | | | 79.00 | | 1F |
| 537.00 | 63.00 | | 256.00 | 75.00 | | 1G |
| 716.00 | 68.00 | | 299.00 | 76.00 | | 1H |
| | 55.00 | | | 76.00 | | 1J |
| | 74.00 | | | 85.00 | | 1K |
| 370.00 | 77.00 | | 99.00 | 88.00 | | 1N |
| | 36.00 | | | 57.00 | | 1O |
| 140.00 | 92.22 | | 38.00 | 92.40 | | 2M |
| | 60.00 | | | 82.00 | | 2AL |
| | 14.00 | | | 57.00 | | 1BI |
| 35.00 | 89.00 | | 18.00 | 94.00 | | 2N |
| | 45.00 | | | 66.00 | | 1U |
| | 51.00 | | | 69.00 | | 1W |
| 588.00 | 83.00 | | 76.00 | | | 1Y |
| 456.00 | 74.00 | | 74.00 | 96.00 | | 1Z |
| 294.50 | 84.00 | | 63.00 | 93.00 | | 2O |
| 115.85 | 91.00 | | 26.00 | 89.00 | | 4A |
| | 58.00 | | | 70.00 | | 1AA |
| 276.00 | 88.00 | | 64.00 | 87.00 | | 2P |
| | 52.00 | | | 73.00 | | 1AB |
| | 58.00 | | | 84.00 | | 1AC |
| | 39.00 | | | 81.00 | | 1AD |
| 32.50 | 92.33 | | 15.50 | 97.00 | | 4B |
| 164.00 | 90.00 | | 80.00 | 93.00 | | 2R |
| | 46.00 | | | 64.00 | | 2S |
| 407.00 | 75.00 | | 76.00 | 91.00 | | 1AE |
| | 26.00 | | | 76.00 | | 1AG |
| | 55.00 | | | 77.00 | | 6D |
| | 36.00 | | | 65.00 | | 2T |
| 142.00 | 69.00 | | 21.00 | 95.00 | | 1AH |
| | 41.00 | | | 71.00 | | 2W |
| 212.00 | 85.00 | | 103.00 | 93.00 | | 2X |
| | 30.00 | | | 67.00 | | 1AI |
| 179.00 | 86.00 | | 122.00 | 90.00 | | 7C |
| 79.50 | 85.50 | | 26.00 | 96.50 | | 2Y |
| | 30.00 | | | 73.00 | | 1AO |
| | 47.00 | | | 84.00 | | 1AS |
| | 28.00 | | | 72.00 | | 6A |
| | 18.00 | | | 28.00 | | 2Z |
| 530.00 | 75.00 | | 65.80 | 90.00 | | 1AT |
| 460.00 | 75.00 | | 287.00 | 84.00 | | 3B |
| 495.00 | 78.00 | | 86.10 | 89.00 | | 1AU |
| | 29.00 | | | 60.00 | | 5A |
| | 62.00 | | | 88.00 | | 1AW |
| | 62.00 | | | 92.00 | | 1AX |
| | 75.00 | | | 92.00 | | 1AY |
| | 66.00 | | | 66.00 | | 8A |
| | 60.00 | | | 74.00 | | 6F |
| | 48.00 | | | 72.00 | | 1AZ |
| | 95.00 | | | 86.00 | | 2AA |
| | 37.00 | | | 82.00 | | 1BA |
| 113.00 | 89.00 | | 23.00 | 97.00 | | 2AB |
| 301.00 | 90.00 | | 53.00 | 95.00 | | 1BB |
| 40.00 | 100.00 | | 32.00 | 97.00 | | 2AC |
| 57.00 | 97.00 | | 17.00 | 99.00 | | 2A |
| 205.00 | 89.00 | | 47.00 | 97.00 | | 1B |
| 219.00 | 87.00 | | 72.00 | 96.00 | | 1BC |
| 289.00 | 84.00 | | 40.00 | 99.00 | | 1BD |
| 57.00 | 96.50 | | 19.00 | 91.00 | | 2AD |
| 812.00 | 75.00 | | 130.00 | 93.00 | | 1BE |
| 110.00 | 94.00 | | 20.00 | 100.00 | | 2AE |
| 167.50 | 92.00 | | 36.00 | 98.00 | | 2AF |
| 517.50 | 76.00 | | 72.00 | 95.00 | | 1BF |
| | 48.00 | | | 84.00 | | 3A |
| 86.00 | 91.00 | | 20.00 | 99.00 | | 2AG |
| 120.00 | 88.00 | | 18.00 | 99.00 | | 2AH |
| | 80.00 | | | 89.00 | | 1BG |
| | 60.00 | | | 75.00 | | 1BH |
| 38.00 | 98.00 | | 21.00 | 100.00 | | 2AI |
| | 84.00 | | | 89.00 | | 2AJ |
| | 37.00 | | | 37.00 | | 1I |
| | 39.00 | | | 32.00 | | 1M |
| | 13.00 | | | 48.00 | | 1S |
| | 20.00 | | | 36.00 | | 1X |
| | 23.00 | | | 33.00 | | 2U |
| | 14.00 | | | 33.00 | | 1AJ |
| | 24.00 | | | 38.00 | | 1AK |
| | 30.00 | | | 33.00 | | 1AM |
| | 34.00 | | | 32.00 | | 7B |
| | 31.00 | | | 56.00 | | 1AN |
| | 19.00 | | | 28.00 | | 10 |
| | 24.00 | | | 47.00 | | 6C |
| 73.00 | 96.00 | | 28.00 | 97.00 | | 6E |
| 5 | | | 4 | | | 11B |
| 16 | | | 4 | | | 11D |
| 32 | | | 39 | | | 6F |
| 23 | | | 15 | | | 6G |
| 2 | | | 26 | | | 2BA |
| 23 | | | 22 | | | 6H |
| 4 | | | 12 | | | 2BB |
| 5 | | | 5 | | | 11G |
| 10 | | | 30 | | | 12C |
| 173 | | | 543 | | | 2BD |
| 13 | | | 149 | | | 2BE |
| 20 | | | 10 | | | 11J |
| 21 | | | 61 | | | 2BF |
| 8 | | | 6 | | | 12A |
| 22 | | | 25 | | | 6I |

The above results show that the compounds according to the present invention are effective inhibitors of the MNK1 and MNK2 activity.

3.3. Analysis of the Main MNK1 and MNK2 Substrate in Response to Cell Treatment with Compounds of the Present Invention

As noted in the background of the invention section, the major substrate of activated MNK kinases is the eukaryotic translation initiation factor 4E (eIF4E). The assay described in the following was used to test whether the phosphorylation of this substrate can be effectively inhibited by compounds of the present invention.

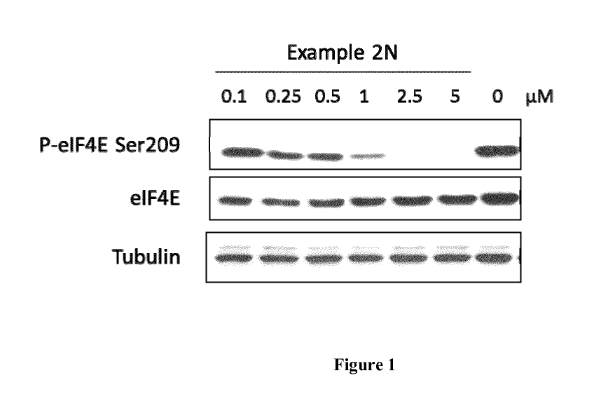

Prostate cancer cells DU145 (1×10⁶ per well) were incubated with compound 2N at concentrations of 0.1, 0.25, 0.5, 1, 2.5 and 5 μM (see also FIG. 1). In a control assay, no compound was added (indicated in FIG. 1 as “0 μM”). The cells were incubated under standard conditions for 6 hours and then lysed. Whole cell lysates were prepared by adding SDS-buffer and the proteins of these lysates were separated using standard SDS-PAGE. A Western Blot was then carried out using standard conditions and the presence of the following proteins in the lysates was detected using the antibodies given in brackets: eIF4E phorphorylated at Ser209 (rabbit anti-human phospho-eIF4E (Ser209) antibody from Cell Signaling #9741), eIF4E (rabbit anti-human eIF4E antibody from Cell Signaling #9742), wherein tubulin served as loading control.

FIG. 1 shows that compound 2N (referred to as example 2N in FIG. 1) is effectively inhibiting eIF4E-phosphorylation in a dose-dependent matter.

3.4. Analysis of IL-6 Expression in Response to Cell Treatment with Compounds of the Present Invention

Autoimmune diseases arise from an inappropriate immune response of the body against substances and tissues normally present in the body. The treatment of autoimmune diseases is typically achieved with immunosuppressive medications which decrease the immune response. In inflammatory diseases, it is the overreaction of the immune system, and its subsequent downstream signaling (TNF, 11-6, etc.), which causes problems. Mitigation of inflammation by activation of anti-inflammatory genes and the suppression of inflammatory genes such as cytokines in immune cells are promising ways of novel therapies. As noted in the background of the invention section, MNK kinases are involved in the expression of proinflammatory cytokines and MNK kinase inhibition results in reduced levels of such cytokines.

The LPS assay is a standard assay to evaluate the ability to respond to an inflammatory stimulus by mounting an acute phase response. The acute phase response is characterized by a dramatic increase in the production of a group of proteins by the liver. Bacterial LPS is an endotoxin, a potent inducer of the acute phase response and systemic inflammation. This response is induced by the production of TNFα, IL-1β, and IL-6 from activated monocytes and neutrophils in response to inflammatory stimuli. Evaluation of one or all of the proinflammatory cytokines TNFα, IL-1β, and IL-6, is the current standard method for evaluation of the ability of the immune system to mount an innate inflammatory immune response.

The ability of the compounds of the present invention to inhibit the production of pro-inflammatory cytokines was tested in the assay described in the following.

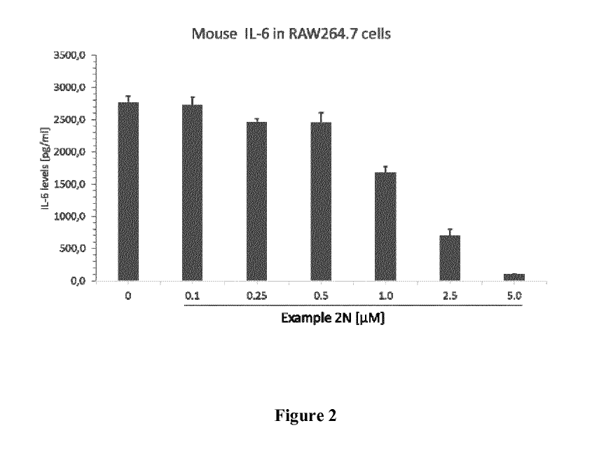

Murine RAW 264.7 leukemic monocyte macrophage cells were plated at the density of 40000 cells per well. At the next day, the cells in each well were pre-incubated with compound 2N to final concentrations of 0.1, 0.25, 0.5, 1.0, 2.5 and 5 μM (see also FIG. 2) for 4 h and then stimulated with LPS (final concentration of 1 μg/ml) for 6 h. The control without compound 2N is indicated as “0 μM”. After this incubation, the IL-6 levels in the cell culture media of each well were examined by ELISA using the BD OptEIA™ Mouse IL-6 ELISA Set Cat. No 555240, according to manufacturer's instructions.

FIG. 2 shows that compound 2N (referred to as example 2N in FIG. 2) is effectively repressing the production of IL-6 in a dose-dependent matter.

1. A compound of formula (I) 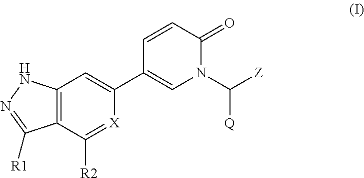
or a salt, stereoisomer, tautomer or N-oxide thereof,
wherein
X is CR3 or N;
R₁ is
(i) H, halogen, OT¹, N(T²)(T³), NHC(O)T⁴, NHS(O)₂T⁴, C(O)OT¹, C(O)N(T²)(T³), C(O)T⁴, ST¹, S(O)₂T⁴, S(O)₂N(T²)(T³), NO₂, or CN; or
(ii) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iv) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T¹, S(O)₂OT⁸ and S(O)₂N(T⁵)(T⁶); or
(v) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(vi) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸, S(O)₂N(T⁵)(T⁶), a ring system according to (ii) above, a ring system according to (iii) above, a ring system according to (iv) above and a ring system according to (v) above;
R₂ and R₃, if present, are independently
(i) H, halogen, OT¹, N(T²)(T³), NHC(O)T⁴, NHS(O)₂T⁴, C(O)OT¹, C(O)N(T²)(T³), C(O)T⁴, ST¹, S(O)₂T⁴, S(O)₂N(T²)(T³), NO₂, or CN; or
(ii) C₁₋₆alkyl, C₃₋₆heteroalkyl, C₂₋₆alkenyl or C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T′)(T⁶), OC(O)N(T⁵)(T⁶), S(O)₂T⁷, S(O)₂OT⁸ and S(O)₂N(T⁵)(T⁶); or
(iii) (C₀₋₆alkyl)T¹⁰, (C₃₋₆heteroalkyl)T¹⁰, (C₂₋₆alkenyl)T¹⁰, (C₂₋₆alkynyl)T¹, (CH₂)ₙO(CH₂)ₙT¹⁰, C(O)(CH₂)ₙT¹⁰, C(O)O(CH₂)ₙT¹⁰, C(O)N(T²)[(CH₂)ₙT¹⁰], NHC(O)(CH₂)ₙT¹⁰, N(T²)[(CH₂)ₙT⁰)], N(T²)[(CH₂)ₙNHT¹⁰], O(CH₂)ₙNHT¹⁰, (CH₂)N(T²)[(CH₂)ₙT¹⁰], (CH₂)ₙS(CH₂)ₙT¹⁰, S(O)₂(CH₂)ₙT¹⁰, S(O)₂O(CH₂)ₙT¹⁰, S(O)₂N(T²)[(CH₂)ₙT¹⁰], NHS(O)₂(CH₂)ₙT¹⁰ or S(CH₂)ₙNHT⁰, wherein n is independently 0, 1, 2, 3 or 4, and wherein T¹⁰ is
a) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆ alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
b) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
c) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
d) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, oxo, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
Z is H, halogen, C₁₋₆alkyl or C₃₋₆heteroalkyl, wherein said C₁₋₆alkyl and C₃₋₆heteroalkyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
Q is
(i) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from
(a) C₁₋₆alkyl, C₃₋₆heteroalkyl, C₂₋₆alkenyl and C₂₋₆alkynyl, wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶), and
(b) halogen, CF₃, OT¹, N(T²)(T³), NHC(O)T⁴, NHC(O)T⁹, NHS(O)₂T⁴, ST¹, S(O)₂T⁴, S(O)₂N(T⁵)(T⁶), NO₂, CN, C(O)H, C(O)OT¹, C(O)N(T²)(T³), C(O)T⁴ and OC(O)N(T²)(T³), and
(c) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆ alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
(d) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
(e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆ alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); and
(f) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(ii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iii) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁸)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(iv) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₃₋₆heteroalkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), wherein said C₁₋₆alkyl, C₃₋₆heteroalkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
T¹, T² and T³ are each independently selected from H, C₁₋₆alkyl and C₃₋₆heteroalkyl, wherein said C₁₋₆alkyl and C₃₋₆heteroalkyl is unsubstituted or independently substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶);
T⁴ is C₁₋₆alkyl or C₃₋₆heteroalkyl, wherein said C₁₋₆alkyl and C₃₋₆heteroalkyl is unsubstituted or independently substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁸, S(O)₂OT⁷ and S(O)₂N(T⁵)(T⁶);
T⁵, T⁶ and T⁷ are each independently selected from H, C₁₋₆alkyl and C₃₋₆heteroalkyl, wherein said C₁₋₆alkyl and C₃₋₆heteroalkyl is unsubstituted or independently substituted with at least one substituent independently selected from halogen, NH₂, NHCH₃, N(CH₃)(CH₃), NO₂, OH, OCH₃, SH, C(O)NH₂, C(O)NHCH₃, C(O)N(CH₃)(CH₃) and CN;
T⁸ is selected from C₁₋₆alkyl and C₃₋₆heteroalkyl, wherein said C₁₋₆alkyl and C₃₋₆heteroalkyl is unsubstituted or independently substituted with at least one substituent independently selected from halogen, NH₂, NHCH₃, N(CH₃)(CH₃), NO₂, OH, OCH₃, SH, C(O)NH₂, C(O)NHCH₃, C(O)N(CH₃)(CH₃) and CN; and
T⁹ is C₂₋₆alkenyl, wherein said C₂₋₆alkenyl is unsubstituted or substituted with at least one substituent independently selected from halogen, N(T⁵)(T⁶), NO₂, OT⁷, ST⁷, CN, C(O)OT⁷, C(O)N(T⁵)(T⁶), OC(O)N(T⁵)(T⁶), S(O)₂T⁸, S(O)₂OT⁷ and S(O)₂N(T⁵)(T⁶).

2. The compound according to claim 1, wherein R₁ is (i) as defined in claim 1 for R₁ or C₁₋₆alkyl, wherein said C₁₋₆alkyl is unsubstituted or substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶).

3. The compound according to claim 1, wherein R1 is (ii) as defined in claim 1 for R1 or (iii) as defined in claim 1 for R1 or (iv) as defined in claim 1 for R1 or (v) as defined in claim 1 for R₁.

4. The compound according to claim 1, wherein X is CR3.

5. The compound according to claim 1, wherein R2 and R3, if present, are independently (i) as defined in claim 1 for R2 and R3, if present; or (ii) as defined in claim 1 for R2 and R3, if present.

6. The compound according to claim 5, wherein R2 and R3, if present, are independently H, halogen, OH, NH₂, NO₂ or unsubstituted C₁₋₆alkyl.

7. The compound according to claim 1, wherein R3 is H, if present, and R2 is (iii) as defined in claim 1 for R2.

8. The compound according to claim 1, wherein Z is H, halogen or C₁₋₆alkyl, wherein said C₁₋₆alkyl is unsubstituted or substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶).

9. The compound according to claim 1, wherein Q is
(i) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from
(a) C₁₋₆alkyl, C₂₋₆alkenyl and C₂₋₆alkynyl, wherein said C₁₋₆alkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶), and
(b) halogen, CF₃, OT¹, N(T²)(T³), NHC(O)T⁴, NHC(O)T⁹, NHS(O)₂T⁴, ST¹, S(O)₂T⁴, NO₂, CN, C(O)H, C(O)OT¹, C(O)N(T²)(T³), C(O)T⁴ and OC(O)N(T²)(T³), and
(c) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); and
(d) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); and
(e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); and
(f) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(ii) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iii) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(iv) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

10. The compound according to claim 9, wherein Q is a monocyclic aromatic heterocyclic ring system with 5 or 6 ring atoms, wherein 1 or 2 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

11. The compound according to claim 9, wherein Q has the following structure 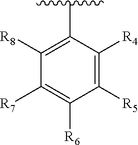
and wherein
R₄, R₅, R₆, R₇ and R₈ are independently
(a) C₁₋₆alkyl, C₂₋₆alkenyl or C₂₋₆alkynyl, wherein said C₁₋₆alkyl, C₂₋₆alkenyl and C₂₋₆alkynyl is unsubstituted or independently substituted with at least one substituent independently selected from halogen, N(T⁵)(T⁶), OT⁷, ST⁷, NO₂, CN, C(O)OT⁷, C(O)N(T⁵)(T⁶), OC(O)N(T⁵)(T⁶), S(O)₂T⁷, S(O)₂OT⁸ and S(O)₂N(T⁵)(T⁶); or
(b) H, halogen, CF₃, OT¹, N(T²)(T³), NHC(O)T⁴, NHC(O)T⁹, NHS(O)₂T⁴, ST¹, S(O)₂T⁴, NO₂, CN, C(O)H, C(O)OT¹, C(O)N(T²)(T³), C(O)T⁴ or OC(O)N(T²)(T³); or
(c) a mono- or bicyclic aromatic heterocyclic ring system with 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(d) a mono- or bicyclic aromatic carbocyclic ring system with 5, 6, 7, 8, 9, 10, 11, 12, 13 or 14 ring carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(e) a monocyclic saturated or partially unsaturated non-aromatic carbocyclic ring system with 3, 4, 5, 6 or 7 carbon atoms, wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³); or
(f) a mono- or bicyclic saturated or partially unsaturated non-aromatic heterocyclic ring system with 3, 4, 5, 6, 7, 8, 9, 10, 11, 12, 13 or 14 ring atoms, wherein 1, 2, 3, 4 or 5 ring atom(s) is/are a hetero atom selected from N, O and S, and the remaining ring atoms are carbon atoms, and wherein said ring system is unsubstituted or substituted with at least one substituent independently selected from C₁₋₆alkyl, C₂₋₆alkenyl, C₂₋₆alkynyl, halogen, CF₃, OT¹, N(T²)(T³), NHC(O)T⁴, NHS(O)₂T⁴, ST¹, S(O)₂T⁴, NO₂, CN, C(O)H, C(O)OT¹, C(O)N(T²)(T³), C(O)T⁴ and OC(O)N(T²)(T³).

12. The compound according to claim 1, wherein said compound is selected from the group consisting of 1-benzyl-5-(1H-indazol-6-yl)-1,2-dihydropyridin-2-one; 1-(2-fluorobenzyl)-5-(1H-indazol-6-yl)pyridin-2(1H)-one; 2-{[5-(1H-indazol-6-yl)-2-oxopyridin-1(2H)-yl]methyl}benzonitrile; 1-(1,3-benzodioxol-5-ylmethyl)-5-(1H-indazol-6-yl)pyridin-2(1H)-one; 1-(2-chlorobenzyl)-5-(1H-indazol-6-yl)pyridin-2(1H)-one; 5-(1H-indazol-6-yl)-1-(4-methoxybenzyl)pyridin-2(1H)-one; 1-(3,4-dichlorobenzyl)-5-(1H-indazol-6-yl)pyridin-2(1H)-one; 5-(1H-indazol-6-yl)-1-(3-methoxybenzyl)pyridin-2(1H)-one; 5-(1H-indazol-6-yl)-1-(3-nitrobenzyl)pyridin-2(1H)-one; 1-(4-fluoro-3-nitrobenzyl)-5-(1H-indazol-6-yl)pyridin-2(1H)-one; 5-(1H-indazol-6-yl)-1-(pyridin-2-ylmethyl)pyridin-2(1H)-one; 1-(3-chlorobenzyl)-5-(1H-indazol-6-yl)pyridin-2(1H)-one; 5-(1H-indazol-6-yl)-1-[(2-methyl-1,3-thiazol-5-yl)methyl]pyridin-2(1H)-one; 5-(1H-indazol-6-yl)-1-[4-(trifluoromethyl)benzyl]pyridin-2(1H)-one; methyl 3-{[5-(1H-indazol-6-yl)-2-oxopyridin-1 (2H)-yl]methyl}benzoate; methyl 2-{[5-(1H-indazol-6-yl)-2-oxopyridin-1(2H)-yl]methyl}benzoate; 5-(1H-indazol-6-yl)-1-(pyridin-4-ylmethyl)pyridin-2(1H)-one; 5-(1H-indazol-6-yl)-1-[2-(trifluoromethyl)benzyl]pyridin-2(1H)-one; 5-(1H-indazol-6-yl)-1-[3-(trifluoromethyl)benzyl]pyridin-2(1H-)-one; 3-{[5-(1H-indazol-6-yl)-2-oxopyridin-1(2H)-yl]methyl}benzonitrile; 3-{[5-(1H-indazol-6-yl)-2-oxopyridin-1(2H)-yl]methyl}benzamide; 5-(1H-indazol-6-yl)-1-(1-phenylethyl)pyridin-2(1H)-one; 5-(1H-indazol-6-yl)-1-[2-(trifluoromethoxy)benzyl]pyridin-2(1H)-one; 5-(1H-indazol-6-yl)-1-[2-(thiophen-2-yl)benzyl]pyridin-2(1H)-one; 1-[3-(difluoromethoxy)benzyl]-5-(1H-indazol-6-yl)pyridin-2(1H)-one; 1-[(5-chlorothiophen-2-yl)methyl]-5-(1H-indazol-6-yl)pyridin-2(1H)-one; 5-(1H-indazol-6-yl)-1-[2-(pyridin-4-yl)benzyl]pyridin-2(1H)-one; 5-(1H-indazol-6-yl)-1-[2-(pyridin-3-yl)benzyl]pyridin-2(1H)-one; 5-(1H-indazol-6-yl)-1-(1H-pyrazol-4-ylmethyl)pyridin-2(1H)-one; 5-(1H-indazol-6-yl)-1-[3-(thiophen-2-yl)benzyl]pyridin-2(1H)-one; 5-(1H-indazol-6-yl)-1-[3-(pyridin-3-yl)benzyl]pyridin-2(1H)-one; 4-{[5-(1H-indazol-6-yl)-2-oxopyridin-1(2H)-yl]methyl}benzonitrile; 1-[3-(hydroxymethyl)benzyl]-5-(1H-indazol-6-yl)pyridin-2(1H)-one; 1-[(4-bromothiophen-2-yl)methyl]-5-(1H-indazol-6-yl)pyridin-2(1H)-one; 5-(1H-indazol-6-yl)-1-(thiophen-3-ylmethyl)pyridin-2(1H)-one; 1-[2-(hydroxymethyl)benzyl]-5-(1H-indazol-6-yl)pyridin-2(1H)-one; 1-[2-(furan-3-yl)benzyl]-5-(1H-indazol-6-yl)pyridin-2(1H)-one; 5-(1H-indazol-6-yl)-1-[2-(thiophen-3-yl)benzyl]pyridin-2(1H)-one; 1-[(2-chloropyridin-4-yl)methyl]-5-(1H-indazol-6-yl)pyridin-2(1H)-one; 5-(1H-indazol-6-yl)-1-[2-(1H-pyrazol-3-yl)benzyl]pyridin-2(1H)-one; 1-(3-chloro-4-fluorobenzyl)-5-(1H-indazol-6-yl)pyridin-2(1H)-one; 1-(3-chlorobenzyl)-5-(1H-indazol-6-yl)pyridin-2(1H)-one; 1-(2-fluoro-3-nitrobenzyl)-5-(1H-indazol-6-yl)pyridin-2(1H)-one; 1-(5-fluoro-2-nitrobenzyl)-5-(1H-indazol-6-yl)pyridin-2(1H)-one; 1-(3-fluoro-2-nitrobenzyl)-5-(1H-indazol-6-yl)pyridin-2(1H)-one; 1-[5-chloro-2-(thiophen-3-yl)benzyl]-5-(1H-indazol-6-yl)pyridin-2(1H)-one; 1-(2-ethenylbenzyl)-5-(1H-indazol-6-yl)pyridin-2(1H)-one; 5-(1H-indazol-6-yl)-1-(tetrahydro-2H-pyran-4-ylmethyl)pyridin-2(1H)-one; 5-(3-amino-1H-indazol-6-yl)-1-(2-fluorobenzyl)pyridin-2(1H)-one; 1-benzyl-5-(3-bromo-1H-indazol-6-yl)pyridin-2(1H)-one; 5-(1H-indazol-6-yl)-1-(2-nitrobenzyl)pyridin-2(1H)-one; 1-(cyclopropylmethyl)-5-(1H-indazol-6-yl)pyridin-2(1H)-one; 5-(1H-indazol-6-yl)-1-(pyridin-3-ylmethyl)pyridin-2(1H)-one; 1-(cyclohexylmethyl)-5-(1H-indazol-6-yl)pyridin-2(1H)-one; 1-benzyl-5-(4-nitro-1H-indazol-6-yl)pyridin-2(1H)-one; 5-(3-amino-1H-indazol-6-yl)-1-benzylpyridin-2(1H)-one; 5-(3-amino-1H-indazol-6-yl)-1-(3-chlorobenzyl)pyridin-2(1H)-one; 5-(3-amino-1H-indazol-6-yl)-1-[2-(trifluoromethyl)benzyl]pyridin-2(1H)-one; 5-(3-amino-1H-indazol-6-yl)-1-[3-(trifluoromethyl)benzyl]pyridin-2(1H)-one; 5-(3-amino-1H-indazol-6-yl)-1-(1-phenylethyl)pyridin-2(1H)-one; 5-(3-amino-5-methyl-1H-indazol-6-yl)-1-benzylpyridin-2(1H)-one; 1-benzyl-5-(1H-pyrazolo[4,3-c]pyridin-6-yl)pyridin-2(1H)-one; 1-benzyl-5-(3-hydroxy-1H-indazol-6-yl)pyridin-2(1H)-one; N-[6-(1-benzyl-6-oxo-1,6-dihydropyridin-3-yl)-1H-indazol-3-yl]acetamide; 1-benzyl-5-(3-methyl-1H-indazol-6-yl)pyridin-2(1H)-one; 5-(3-amino-1H-indazol-6-yl)-1-[(5-chlorothiophen-2-yl)methyl]pyridin-2(1H)-one; 1-benzyl-5-(5-nitro-1H-indazol-6-yl)pyridin-2(1H)-one; 5-(3-amino-1H-indazol-6-yl)-1-(thiophen-3-ylmethyl)pyridin-2(1H)-one; 5-(3-amino-1H-indazol-6-yl)-1-[2-(thiophen-2-yl)benzyl]pyridin-2(1H)-one; N-(3-{[5-(3-amino-1H-indazol-6-yl)-2-oxopyridin-1(2H)-yl]methyl}phenyl)acetamide; 5-(3-amino-1H-indazol-6-yl)-1-(3-fluorobenzyl)pyridin-2(1H)-one; 5-(3-amino-1H-indazol-6-yl)-1-(2-fluoro-3-nitrobenzyl)pyridin-2(1H)-one; 5-(3-amino-1H-indazol-6-yl)-1-(5-fluoro-2-nitrobenzyl)pyridin-2(1H)-one; 5-(3-amino-1H-indazol-6-yl)-1-(3-chloro-4-fluorobenzyl)pyridin-2(1H)-one; 5-(3-amino-1H-indazol-6-yl)-1-(3-fluoro-2-nitrobenzyl)pyridin-2(1H)-one; 5-(3-amino-1H-indazol-6-yl)-1-[5-chloro-2-(thiophen-3-yl)benzyl]pyridin-2(1H)-one; 5-(3-amino-1H-indazol-6-yl)-1-(2-ethenylbenzyl)pyridin-2(1H)-one; 5-(3-amino-1H-indazol-6-yl)-1-(2-ethenylbenzyl)pyridin-2(1H)-one; 5-(4-amino-1H-indazol-6-yl)-1-benzylpyridin-2(1H)-one; 1-benzyl-5-[3-(pyridin-4-yl)-1H-indazol-6-yl]pyridin-2(1H)-one; 1-benzyl-5-[3-(4-hydroxyphenyl)-1H-indazol-6-yl]pyridin-2(1H)-one; 1-benzyl-5-[3-(3-methoxyphenyl)-1H-indazol-6-yl]-1,2-dihydropyridin-2-one; 1-benzyl-5-[3-(3-methylphenyl)-1H-indazol-6-yl]-1,2-dihydropyridin-2-one; N-(3-{[5-(3-amino-1H-indazol-6-yl)-2-oxo-1,2-dihydropyridin-1-yl]methyl}phenyl)-2-fluoroacetamide; N-(3-{[5-(3-amino-1H-indazol-6-yl)-2-oxo-1,2-dihydropyridin-1-yl]methyl}-4-fluorophenyl)acetamide; N-(2-{[5-(3-amino-1H-indazol-6-yl)-2-oxo-1,2-dihydropyridin-1-yl]methyl}phenyl)acetamide; N-(3-{[5-(3-amino-1H-indazol-6-yl)-2-oxo-1,2-dihydropyridin-1-yl]methyl}-2-fluorophenyl)acetamide; 2-{[5-(3-amino-1H-indazol-6-yl)-2-oxo-1,2-dihydropyridin-1-yl]methyl}benzene-1-sulfonamide; 1-[2-(bromomethyl)benzyl]-5-(1H-indazol-6-yl)pyridin-2(1H)-one; 1-[3-(bromomethyl)benzyl]-5-(1H-indazol-6-yl)pyridin-2(1H)-one; 1-(3-hydroxybenzyl)-5-(1H-indazol-6-yl)pyridin-2(1H)-one; 5-(3-amino-1H-indazol-6-yl)-1-(3-hydroxybenzyl)pyridin-2(1H)-one; 2-{[5-(1H-indazol-6-yl)-2-oxopyridin-1(2H)-yl]methyl}benzamide; 1-{4-[(2-aminoethyl)amino]-3-nitrobenzyl}-5-(1H-indazol-6-yl)pyridin-2(1H)-one hydrochloride; 1-(3-aminobenzyl)-5-(1H-indazol-6-yl)pyridin-2(1H)-one hydrochloride; 1-[(6-aminopyridin-2-yl)methyl]-5-(1H-indazol-6-yl)pyridin-2(1H)-one hydrochloride; 3-amino-N-(3-{[5-(1H-indazol-6-yl)-2-oxopyridin-1(2H)-yl]methyl}phenyl)propanamide hydrochloride; 5-[3-amino-4-({[(1R,4R)-4-aminocyclohexyl]methyl}amino)-1H-indazol-6-yl]-1-[(3-chlorophenyl)methyl]-1,2-dihydropyridin-2-one hydrochloride; tert-butyl 4-{[(3-amino-6-{1-[(3-chlorophenyl)methyl]-6-oxo-1,6-dihydropyridin-3-yl}-1H-indazol-4-yl)amino]methyl}piperidine-1-carboxylate hydrochloride; 5-[3-amino-4-(piperidin-4-ylmethoxy)-1H-indazol-6-yl]-1-[(3-chlorophenyl)methyl]-1,2-dihydropyridin-2-one hydrochloride; 5-{3-amino-4-[(pyrrolidin-3-ylmethyl)amino]-1H-indazol-6-yl}-1-[(3-chlorophenyl)methyl]-1,2-dihydropyridin-2-one hydrochloride; 5-{3-amino-4-[(piperidin-4-yl)amino]-1H-indazol-6-yl}-1-[(3-chlorophenyl)methyl]-1,2-dihydropyridin-2-one hydrochloride; N-(3-{[5-(1H-indazol-6-yl)-2-oxopyridin-1(2H)-yl]methyl}phenyl)acetamide; N-(3-{[5-(1H-indazol-6-yl)-2-oxopyridin-1(2H)-yl]methyl}phenyl)prop-2-enamide; (2Z)-4-(dimethylamino)-N-(2-{[5-(1H-indazol-6-yl)-2-oxo-1,2-dihydropyridin-1-yl]methyl}phenyl)but-2-enamide; 1-(2-aminobenzyl)-5-(1H-indazol-6-yl)pyridin-2 (1H)-one; 5-(5-amino-1H-indazol-6-yl)-1-benzylpyridin-2(1H)-one; 5-{3-amino-4-[(oxan-4-ylmethyl)amino]-1H-indazol-6-yl}-1-[(3-chlorophenyl)methyl]-1,2-dihydropyridin-2-one; 5-{3-amino-4-[(oxolan-3-ylmethyl)amino]-1H-indazol-6-yl}-1-[(3-chlorophenyl)methyl]-1,2-dihydropyridin-2-one; 5-(4-{[(1-acetylpiperidin-4-yl)methyl]amino}-3-amino-1H-indazol-6-yl)-1-[(3-chlorophenyl)methyl]-1,2-dihydropyridin-2-one; N-[3-({5-[3-amino-4-(oxan-4-ylmethoxy)-1H-indazol-6-yl]-2-oxo-1,2-dihydropyridin-1-yl}methyl)phenyl]acetamide; 5-(4-{[(1-acetylpiperidin-3-yl)methyl]amino}-3-amino-1H-indazol-6-yl)-1-[(3-chlorophenyl)methyl]-1,2-dihydropyridin-2-one; and 5-[3-amino-4-(oxan-4-ylmethoxy)-1H-indazol-6-yl]-1-[(3-chlorophenyl)methyl]-1,2-dihydropyridin-2-one, or a salt, stereoisomer, tautomer or N-oxide thereof.

13. A pharmaceutical composition, comprising a compound according to claim 1, or a salt, stereoisomer, tautomer or N-oxide thereof.

14. A method of treating a disease selected from the group consisting of an oncogenic, a metabolic, an inflammatory, an autoimmune and a viral disease, comprising administering to a patient in need thereof the pharmaceutical composition as defined in claim 13.

15. A method of inhibiting the activity of MNK1 or MNK2 kinase, or both MNK1 and MNK2 kinases, comprising exposing said MNK1 or MNK2 kinase, or both MNK1 and MNK2 kinases, to at least one compound as defined in claim 1, or a salt, stereoisomer, tautomer or N-oxide thereof.

16. The method according to claim 15, wherein said method is performed outside a human or animal body.

17. A method of treating a disease selected from the group consisting of an oncogenic, a metabolic, an inflammatory, an autoimmune and a viral disease, comprising administering to a patient in need thereof a pharmaceutically effective amount of at least one compound as defined in claim 1, or a salt, stereoisomer, tautomer or N-oxide thereof.
